(19) United States
(12) Patent Application Publication
(10) Pub. No.: US 2023/0128194 A1
(43) Pub. Date: Apr. 27, 2023

(54) METHODS AND COMPOSITIONS TO ALTER HEPATIC GABA RELEASE TO TREAT OBESITY-RELATED CONDITIONS

(71) Applicant: ARIZONA BOARD OF REGENTS ON BEHALF OF THE UNIVERSITY OF ARIZONA, Tucson, AZ (US)

(72) Inventors: Benjamin J. Renquist, Tucson, AZ (US); Caroline E. Geisler, Tucson, AZ (US)

(21) Appl. No.: 17/937,604

(22) Filed: Oct. 3, 2022

Related U.S. Application Data
(63) Continuation-in-part of application No. PCT/US2021/025629, filed on Apr. 2, 2021.
Application No. PCT/US2021/025629 is a continuation-in-part of application No. PCT/US2020/052571, filed on Sep. 24, 2020.
Application No. PCT/US2020/052571 is a continuation-in-part of application No. 16/617,108, filed on Nov. 26, 2019.
(60) Provisional application No. 63/004,373, filed on Apr. 2, 2020.
(60) Provisional application No. 62/647,468, filed on Mar. 23, 2018.
(60) Provisional application No. 62/511,753, filed on May 26, 2017.

(51) Int. Cl. A61K 31/7125; A61P 3/04

(52) U.S. Cl. CPC A61K 31/7125; A61P 3/04

(57) ABSTRACT

Methods and compositions for treating conditions caused by altered hepatic GABA production and release, including hyperinsulinemia, insulin resistance, type II diabetes, obesity, and obesity-related conditions. The present invention describes hepatic GABA as a hepatokine. The methods herein feature manipulating the expression and/or activity of specific GABA transporters, e.g., increasing expression of SLC6A12 and/or SLC6A13 genes or increasing activity of the proteins for which they encode, BGT1 and/or GAT2; or decreasing expression of SLC6A6 and SLC6A8 genes or increasing the activity of the proteins for which they encode, TauT and/or CRT, which can increase hepatic GABA re-uptake or decrease hepatic GABA release to improve insulin sensitivity and prevent hypertension.

### CROSS REFERENCE

This application is a Continuation-In-Part and claims benefit of PCT/US2021/025629 filed Apr. 2, 2021, which claims benefit of PCT/US2020/052571 filed Sep. 24, 2020 and U.S. Provisional Application No. 63/004,373 filed Apr. 2, 2020, the specification(s) of which is/are incorporated herein in their entirety by reference.

This application is also a Continuation-In-Part and claims benefit of U.S. application Ser. No. 16/617,108 filed Nov. 26, 2019, which is a 371 and claims benefit of PCT/US2018/034680 filed May 25, 2018, which claims benefit of U.S. Provisional Application No. 62/647,468 filed Mar. 23, 2018 and U.S. Provisional Application No. 62/511,753 filed May 26, 2017, the specification(s) of which is/are incorporated herein in their entirety by reference.

### BACKGROUND OF THE INVENTION

### Field of the Invention

The present invention relates to methods and compositions for treating obesity-related conditions such as type II diabetes, insulin resistance, hyperinsulinemia, hypertension, hyperphagia, and obesity-related conditions. More particularly, the present invention describes but is not limited to methods and compositions for treating conditions caused by altered hepatic GABA production and release, including obesity, hyperinsulinemia and insulin resistance, wherein manipulating the expression and/or activity of specific GABA transporters (e.g., increasing expression of SLC6A12 and/or SLC6A13 genes or increasing activity of the proteins for which they encode, BGT1 and/or GAT2; or decreasing expression of SLC6A6 and SLC6A8 genes or increasing the activity of the proteins for which they encode, TauT and/or CRT) can increase hepatic GABA re-uptake or decrease hepatic GABA release to improve insulin sensitivity and prevent hypertension. The present invention also describes methods and systems for modulating hepatic GABA production and release or hepatic vagal afferent nerve signaling to alter food intake and/or body weight.

### Background Art

Type II diabetes (T2D) affects 30 million Americans, while an additional 81 million Americans have pre-diabetes. Thus, 46% of the U.S. adult population is affected by diabetes, the 7^{th} leading cause of death in America, which consumes 1 in every 7 U.S. health care dollars. The high prevalence, mortality, and economic burden of T2D underscores a critical need for the development of additional therapeutics to treat diabetes.

Furthermore, 87% of diabetics are overweight or obese. Obesity and T2D are also characterized by dysregulated energy homeostasis, particularly diminished meal-induced satiety which can result in excessive energy intake. Interestingly, hepatic lipid accumulation is directly associated with increased energy intake. In individuals with non-alcoholic fatty liver disease (NAFLD), the percent hepatic steatosis positively correlates with total energy intake and carbohydrate intake. Hepatic lipid accumulation (hepatic steatosis) is a hallmark of T2D and is associated with obesity-induced hyperinsulinemia, insulin resistance, and hyperphagia. Hepatic synthesis of GABA, catalyzed by GABA-transaminase (GABA-T), is upregulated in obese mice. The degree of hepatic lipid accumulation in obesity correlates with the severity of hyperinsulinemia and systemic insulin resistance.

The liver produces and releases hepatokines into circulation in response to acute and chronic nutrient status. For example, FGF21 and ANGPTL4 are secreted from hepatocytes in response to liver nutrient flux and can act in an endocrine fashion to impact whole body metabolism. The present invention establishes that obesity-induced hepatic lipid accumulation increases hepatocyte production and release of the inhibitory neurotransmitter, GABA, in mice that acts in a paracrine fashion to decrease the firing activity of the hepatic vagal afferent nerve (HVAN), resulting in increased insulin secretion and decreased skeletal muscle glucose clearance. These findings provide a novel mechanistic link explaining how hepatic lipid accumulation, through increasing liver production and release of GABA (e.g., hepatokine), impairs HVAN signaling, driving the development of metabolic diseases.

The literature suggests that the hepatic vagal nerve communicates with the central nervous system to affect pancreatic insulin release and peripheral tissue insulin sensitivity. The HVAN regulates parasympathetic efferent nerve activity at the pancreas to alter insulin secretion. A decrease in HVAN firing frequency stimulates insulin secretion, whereas an increase in HVAN firing frequency decreases insulin secretion. The HVAN is also involved in regulating whole-body insulin sensitivity. Hepatic vagotomy diminishes insulin sensitivity (assessed as insulin-stimulated glucose uptake) in insulin sensitive rats, while improving insulin sensitivity and glucose tolerance in insulin resistant mice. Therefore, the firing frequency of the HVAN is integral to controlling insulin secretion and sensitivity.

In addition to serving as a hub of metabolism, the liver is also a key endocrine organ which produces a significant number of hepatokines that are altered by obesity, NAFLD, and exercise and signal to change metabolic function in other tissues. Despite the established role of the HVAN in affecting both insulin release and sensitivity, none of these hepatokines have been implicated in altering hepatic vagal afferent nerve activity. Hepatocellular lipid accumulation depolarizes hepatocytes. Because NAFLD is integral to the development of hyperinsulinemia and insulin resistance and the HVAN regulates insulin secretion and action, a hypothesis was tested that lipid-induced hepatocyte depolarization changes the release of neurotransmitters to affect firing activity of the HVAN and drive the dysregulation of systemic glucose homeostasis common in obesity.

The present invention shows that hepatic steatosis dysregulates glucose and insulin homeostasis. Obesity-induced hepatocellular lipid accumulation results in hepatocyte depolarization. The present invention shows that hepatocyte depolarization depresses hepatic afferent vagal nerve firing, increases GABA release from liver slices, and causes hyperinsulinemia. Preventing hepatic GABA release or eliminating the ability of the liver to communicate to the hepatic vagal nerve ameliorates the hyperinsulinemia and insulin resistance associated with diet-induced obesity. In people with obesity, hepatic expression of GABA transporters is associated with basal serum insulin, hepatic insulin sensitivity index, and glucose infusion and disposal rates during a hyperinsulinemic euglycemic clamp. Single nucleotide polymorphisms in hepatic GABA re-uptake transporters are associated with an increased incidence of D2M.

The present invention features a new use of GABA as a novel hepatokine that is dysregulated in obesity and whose release can be manipulated to mute or exacerbate the glucoregulatory dysfunction common to obesity. The present invention describes the use of four GABA transporters whose activity can be manipulated to alter hepatic slice GABA release. Hepatic expression of SLC6A12 and SLC6A13 is positively correlated with insulin sensitivity. The present invention shows that inhibiting these two transporters increases liver slice media GABA concentrations by preventing re-uptake. Hepatic expression of SLC6A6 and SLC6A8 is negatively associated with insulin sensitivity, proposing that these two transporters export hepatic GABA. The present invention utilizes pharmacological agents that increase expression or activity of SLC6A12 and/or SLC6A13 or pharmacological agents that inhibit expression or activity of SLC6A6 and/or SLC6A8 to improve insulin sensitivity and prevent hypertension.

The findings described herein, provide a novel mechanistic link explaining how hepatic lipid accumulation, though increasing extracellular hepatocyte-produced GABA to impair HVAN signaling, drives the development of metabolic diseases.

### BRIEF SUMMARY OF THE INVENTION

The present invention provides compositions and methods for decreasing hepatic GABA release or increasing hepatic GABA re-uptake to treat obesity and obesity-related conditions (e.g., hyperphagia, hypertension, insulin resistance, and hyperinsulinemia) as specified in the independent claims.

Embodiments of the present invention can be freely combined with each other if they are not mutually exclusive.

For example, the present invention features a method of treating obesity and/or obesity-related conditions in a subject in need thereof. In some embodiments, the method comprises administering to the subject a therapeutic amount of a composition for altering GABA release by increasing hepatic expression or activity of GABA transporters encoded for by SLC6A12 and/or SLC6A13, or by inhibiting hepatic expression or activity of GABA transporters encoded for by SLC6A6 and/or SLC6A8. This altering of activity of specific hepatic GABA transporters decreases GABA release. The methods herein may help improve insulin sensitivity, decrease body mass, cause weight loss, decrease food intake, and/or prevent hypertension.

A non-limiting example of the present invention describes 1) increasing the liver expression of SLC6A12 and/or SLC6A13 or activity of their proteins (BGT1 and GAT2, respectively) improves GABA re-uptake and/or 2) decreasing the liver expression of SLC6A6 and/or SLC6A8 or activity of their proteins (TauT and CRT, respectively) prevents GABA release. Either improving GABA re-uptake or preventing GABA release will help to prevent obesity, insulin resistance, hypertension, hyperinsulinemia, and/or hyperphagia.

The present invention also features a method for improving insulin sensitivity in a subject in need thereof. In some embodiments, the method comprises: administering to the subject a therapeutic amount of a composition for altering GABA release by increasing hepatic expression or activity of GABA transporters, SLC6A12 and/or SLC6A13, or by inhibiting hepatic expression or activity of GABA transporters, SLC6A6 and/or SLC6A8. This altering of activity of specific hepatic GABA transporters alters GABA re-uptake and release to improve insulin sensitivity, decrease body mass, cause weight loss, decrease food intake, and/or prevent hypertension.

The present invention also features a pharmaceutical composition, for example for use in the methods described herein such as but not limited to methods for treating an obesity-related condition. In some embodiments, the composition comprises an activator or stimulator of hepatic expression of GABA transporters, SLC6A12 and/or SLC6A13, or activity of their proteins, BGT1 and GAT2, to improve GABA re-uptake, or an inhibitor of GABA transporters, SLC6A6 and/or SLC6A8 or activity of their proteins, TauT and/or CRT to prevent GABA release. The composition may be effective for decreasing blood glucose, decreasing blood insulin, improving insulin sensitivity, increasing glucose tolerance, and decreasing/normalizing blood pressure or a combination thereof.

The present invention also features a method of causing a subject in need thereof to lose weight. In some embodiments, the method comprises administering to the patient a therapeutic amount of a composition for altering hepatic activity and/or expression of GABA transporters. In some embodiments, the composition is for increasing hepatic expression of genes encoding GABA transporters, SLC6A12 and/or SLC6A13, or for decreasing hepatic expression of genes encoding GABA transporters, SLC6A6 and/or SLC6A8. In certain embodiments, the composition is for increasing expression or activity of the GABA transporters, BGT1 and/or GAT2, or decreasing expression of activity of the GABA transporters, TauT and/or CRT, decreasing hepatic GABA synthesis or hepatic GABA release or increasing hepatic GABA re-uptake. In some embodiments, altering hepatic activity and/or expression of GABA transporters, SLC6A12, SLC6A13, SLC6A6, SLC6A8, causes a decrease in food intake so that the subject loses weight and/or adiposity. The present invention features altering food intake by regulating GABA release. For example, the present invention features methods and compositions for inducing weight loss (reducing food intake) by depressing hepatic GABA release. The present invention also features methods and compositions for increasing weight gain by increasing food intake through enhancing hepatic GABA production or release.

In some embodiments, the present invention features methods and compositions for treating obesity-related conditions by regulating glucose homeostasis. Briefly, the methods and compositions herein may feature limiting hepatic mitochondrial uncoupling, decreasing hepatic GABA release, hyperpolarizing the hepatocyte, and preventing obesity induced depolarization of the hepatocyte membrane potential. More specifically, the methods may feature inhibitors for GABA synthesis and/or inhibitors for GABA release, e.g., inhibitors for GABA-T, TauT (GABA transporter), or CRT (GABA transporter). The methods and compositions herein may be used for a variety of purposes including but not limited to treating obesity, type 2 diabetes, insulin resistance, hyperinsulinemia, hypertension, and/or hyperphagia.

In other embodiments, the present invention may be used for altering food intake by regulating GABA production or GABA release. For example, the present invention features methods and compositions for losing weight (reducing food intake) by depressing hepatic GABA production or release. The present invention also features methods and compositions for gaining weight by increasing food intake, a result of enhanced hepatic GABA production or release.

In some embodiments, the composition normalizes blood pressure, reduces blood glucose, improves glucose homeostasis in obesity, improves obesity-induced metabolic dysfunction, decreases body mass and fat mass, and/or reduces food intake in obesity.

The present invention features methods of reducing food intake in a monogastric animal (e.g., pig, chicken, dog, cat, horse, rodent, e.g., mouse, rat, etc.) or a human. In some embodiments, the method comprises administering to the monogastric animal or human an effective amount of a composition that depresses hepatic GABA production or release, wherein depressing hepatic GABA production or release causes the monogastric animal or human to reduce its food intake as compared to its food intake prior to being administered the composition. The method may be applied for weight loss purposes.

In certain embodiments, the composition inhibits GABA signaling on the hepatic vagal afferent nerve.

In certain embodiments, the composition inhibits expression of or activity of GABA transaminase (GABA T) or inhibits GABA production. In certain embodiments, the composition that inhibits expression of or activity of GABA transaminase, or inhibits GABA production comprises valproic acid, vigabatrin, phenylethylidenehydrazine (PEH), ethanolamine-O-sulfate (EOS), L-cycloserine, aminooxyacetic acid, gabaculine, phenelzine, rosmarinic acid, branched chain fatty acid, 2-methyl, 2-ethylcaproic acid, 2,2-dimethylvaleric acid, S-vigabatrin, [3-(aminomethyl)phenyl]acetic acid, [2-(aminomethyl)phenyl]acetic acid, ursolic acid, succinic semialdehyde, succinate, Sr2+, SH-group reagent, pyruvate, propionic acid, pimelic acid, phenylhydrazine, oxalacetate, ornithine, oleanolic acid, Ni2+, muscimol, monoiodoacetate, Mn2+, Mg2+, methanol, maleate, lysyl reagents, KCN, imperatorin, hydroxylamine, hydrazine, HgCl2, glyoxylate, glycine, glutarate, glutamic acid, GDP, gastrodigenin, gamma-vinyl 4-aminobutanoate, gabaculine, falcarindiol, ethylamine-2-sulfonic acid, ethanol, trimethylcitryl-beta-D-galactopyranoside, tetrazole-5-(alpha-vinyl-propanamine), propan-2-one N-(2,4-dimethylphenyl)semicarbazone, p-chloromercuribenzoate, N-(4-bromophenyl)-3-(4-fluorophenyl)-6,7-dimethoxy-3a,4-dihydroindeno[1,2-c]pyrazole-2(3H)-carboxamide, N-(4-bromophenyl)-3-(4-chlorophenyl)-6,7-dimethoxy-3a,4-dihydroindeno[1,2-c]pyrazole-2(3H)-carboxamide, DL-cysteine, divalent metal ions, dioxan, D-penicillamine, D-cycloserine, cycloserine, Cu2+, Co2+, Cd2+, Ca2+, carbonyl reagents, butyric acid, beta-alanine, beta-cypermethrin, baclofen, Ba2+, ATP, aminooxyacetate, alpha-alanine, ADP, adipic acid, acetic acid, 6-Azauracil, 6-Azathymine, 5-thiouracil, 5-nitrouracil, DL-3-amino-1-cyclopentene-1-carboxylic acid, DL-trans-4-amino-2-cyclopentene-1-carboxylic acid, cis-3-aminocyclohex-4-ene-1-carboxylic acid, 5-iodouracil, 5-diazouracil, (+/−)-(1S,2R,4S,5S)-4-amino-6,6-difluorobicyclo[3.1.0]hexane-2-carboxylic acid, (+/−)-(1S,2S,4S,5S)-4-amino-6,6-difluorobicyclo[3.1.0]hexane-2-carboxylic acid, (+/−)piperidine-3-sulfonic acid, (1R,3S,4S)-3-amino-4-fluorocyclopentane-1-carboxylic acid, (1R,4S)-4-amino-2-cyclopentene-1-carboxylic acid, (1R,4S)-4-amino-3-fluorocyclopent-2-enecarboxylic acid, (1R,4S)-4-amino-3-pentafluoroethylcyclopent-2-enecarboxylic acid, (1R,4S)-4-amino-3-trifluoromethylcyclopent-2-enecarboxylic acid, (1S,2S,3E)-2-amino-3-(fluoromethylidene)cyclopentanecarboxylic acid, (1S,2S,3Z)-2-amino-3-(fluoromethylidene)cyclopentanecarboxylic acid, (1S,3S)—(Z)-3-amino (2,2,2-trifluoroethylidene)cyclopentanecarboxylic acid, (1S,3S)-3-amino-4-(2,2,2-trifluoro trifluoromethylethylidene)-cyclopentanecarboxylic acid, (1S,3S)-3-ami no difluoromethylenecyclopentanecarboxylic acid, (1S,4R)-4-amino-2-cyclopentene-1-carboxylic acid, (1S,4S)-2-(difluoromethylidene)-4-(1H-tetrazol-5-yl)cyclopentanamine, (2E)-4-methylpentan-2-one N-(2,4-dimethylphenyl)semicarbazone, (2E)-butan-2-one N-(2,4-dimethylphenyl)semicarbazone, (4R)-4-amino-1-cyclopentene-1-carboxylic acid, (4S)-4-amino-1-cyclopentene-1-carboxylic acid, (R,S)-4-amino-3-fluorobutanoic acid, (S)-4-amino-4,5-dihydro-2-thiophenecarboxylic acid, (Z)-4-amino-2-butenoic acid, 1-(4-acetylphenyl)-3-(4-bromophenyloxy)-pyrrolidine-2,5-dione, 1-(4-acetylphenyl)-3-(salicyldehydoxy)-pyrrolidine-2,5-dione, 1H-tetrazole-5-(alpha-vinyl-propanamine), 2,4-diaminobutanoate, 2,4-dimethylphenyl semicarbazide hydrochloride, 2-Aminobenzenesulfonate, 2-aminobutanoate, 2-aminoethane phosphonic acid, 2-N-(acetylamino)cyclohexane sulfonic acid, 2-oxoadipic acid, 2-oxoglutarate, 2-Thiouracil, 3-(aminomethyl)benzoic acid, 3-aminocyclohexanecarboxylic acid, 3-chloro-1-(4-hydroxyphenyl)propan-1-one, 3-Chloro-4-aminobutanoate, 3-Mercaptopropionic acid, 3-Methyl-2-benzothiazolone hydrazone hydrochloride, 3-Phenyl-4-aminobutanoate, 4-(aminomethyl)-1H-pyrrole-2-carboxylic acid, 4-(aminomethyl)furan-2-carboxylic acid, 4-(aminomethyl)furan-3-carboxylic acid, 4-(aminomethyl)thiophene-2-carboxylic acid, 4-(aminomethyl)thiophene-3-carboxylic acid, 4-acryloylphenol, 4-amino-2-fluorobutanoate, 4-amino-5-fluoropentanoic acid, 4-Amino-hex-5-enoic acid, 4-aminohex-5-enoic acid, 4-Aminohex-5-ynoic acid, 4-ethynyl-4-aminobutanoate, 4-hydroxybenzaldehyde, 4-hydroxybenzylamine, 5,5′-dithiobis-2-nitrobenzoic acid, 5-(aminomethyl)-1H-pyrrole-2-carboxylic acid, 5-(aminomethyl)furan-2-carboxylic acid, 5-(aminomethyl)thiophene-2-carboxylic acid, L-tyrosine, L-DOPA, desipramine, timonacic, amicar, orindyl, pemirolast, mesalazine, glutathione, gly-gly, 5-ALA, Vit. U, methionine, glutamine, pyridoxalphosphate, acivicin, GABOB, 3-ABA, 5-AVA, glycine, carnitine, amitriptyline, pregabalin, erythromycin, cyclosporin A, rifampicin, EF1502, betaine, and NNC 05-2090 hydrochloride, 5-amino-1,3-cyclohexadienylcarboxylate, and metformin. In certain embodiments, the composition that inhibits expression of or activity of GABA transaminase or inhibits GABA production is an AMPK activator.

In certain embodiments, the composition inhibits GABA release. In certain embodiments, the composition inhibits expression or activity of GABA transporters that export hepatic GABA. In certain embodiments, the composition that inhibits GABA release inhibits mRNA or protein expression of the Solute Carrier Family 6 Member 6 (SLC6A6) gene or the Solute Carrier Family 6 Member 8 (SLC6A8) gene. In certain embodiments, the composition inhibits mRNA or protein expression of or activity of TauT, a GABA transporter protein encoded by the SLC6A6 gene. In certain embodiments, the composition inhibits mRNA or protein expression of or activity of creatine transporter (CRT), a GABA transporter protein encoded by SLC6A8 gene. In certain embodiments, the composition that inhibits mRNA or protein expression or activity of SCL6A6 or TauT is vigabatrin, δ-ALA, guvacine, taurine, Beta-alanine, Guanidinoacetate, β-Guanidinopropionate, γ-Guanidinobutyrate, Guanidinoethansulfonate, and taurine. In certain embodiments, the composition that inhibits expression or activity of SCL6A8 or CRT is an AMPK activator, Guanidinoacetate, β-Guanidinopropionate, γ-Guanidinobutyrate, Guanidinoethansulfonate, creatinine, methylguanidine, I-arginine, RGX-202, 2,4-dinitro-1-fluorobenzene, tetraethylammonium, guanidine, creatine, arginine, lysine, DTBM, DNFB, or NEM.

In certain embodiments, the composition improves GABA re-uptake. In certain embodiments, the composition increases mRNA or protein expression of the Solute Carrier Family 6 Member 12 (SLC6A12) gene or the Solute Carrier Family 6 Member 13 (SLC6A13) gene. In certain embodiments, the composition increases mRNA or protein expression of or activity of BGT1, a GABA transporter protein encoded by the SLC6A12 gene. In certain embodiments, the composition increases mRNA or protein expression of or activity of GAT2, a GABA transporter protein encoded by the SLC6A13 gene. In certain embodiments, the composition that increases expression of SLC6A12 and/or SLC6A13 is an AMPK activator.

In certain embodiments, the composition inhibits expression or activity of succinate semialdehyde dehydrogenase. In certain embodiments, the composition that inhibits expression or activity of succinate semialdehyde dehydrogenase comprises 2-methyl, 2-ethylcaproic acid; 2,2-dimethylvaleric acid, 2-oxoglutaric semialdehyde, 4-dimethylaminoazobenzene-4-iodoacetamide, 4-hydroxy-trans-2-nonenal, 4-hydroxybenzaldehyde, 4-methoxybenzaldehyde, 4-tolualdehyde, 5,5′-dithiobis(2-nitrobenzoic acid), Acetaldehyde, Acrolein, ADP, AMP, Arsenite, ATP, Benzaldehyde, Ca2+, Cd2+, Chloral hydrate, Cu2+, Disulfiram, Dithionitrobenzoate, Fe3+, Glyoxylate, Hg2+, Iodoacetamide, m-hydroxybenzaldehyde, Mg2+, Mn2+, N-ethylmaleimide, N-formylglycine, NAD+, NADH, NEM, Ni2+, o-phthalaldehyde, p-bromobenzaldehyde, p-chlorobenzaldehyde, p-chloromercuriphenyl sulfonate, p-ethoxybenzaldehyde, p-ethylbenzaldehyde, p-fluorobenzaldehyde, p-hydroxymercuribenzoate, p-iodobenzaldehyde, p-isopropylbenzaldehyde, p-Methoxybenzaldehyde, p-methylbenzaldehyde, p-nitrobenzaldehyde, Pb2+, PCMB, pyridoxal 5′-phosphate, succinate semialdehyde, Valeraldehyde, and Zn2+. In certain embodiments, the composition that inhibits expression or activity of succinate semialdehyde dehydrogenase is an AMPK activator.

In certain embodiments, the AMPK activator is a biguanide, a thiazolidinedione, a ginsenoside, or a polyphenol. In certain embodiments, the AMPK activator is A-769662, metformin, resveratrol, troglitazone, pioglitazone, rosiglitazone, quercetin, genistein, epigallocatechin gallate, berberine, curcumin, ginsenoside Rb1, alpha-lipoic acid, cryptotanshinone, 5-aminoimidazole-4-carboxaminde ribonucleoside (AICAR), benzimidazole, salicylate, compound-13, PT-1, MT63-78, and APC.

In certain embodiments, the composition comprises an inhibitor of sodium potassium ATPase. In certain embodiments, the composition reduces hepatic mitochondrial uncoupling.

The present invention features methods of increasing food intake in a monogastric animal. In some embodiments, the method comprises administering to the monogastric animal an effective amount of a composition that increases hepatic GABA production or release, wherein increasing hepatic GABA production or release causes the monogastric animal to increase its food intake as compared to its food intake prior to being administered the composition. The method may be applied for improving weight gain. In certain embodiments, the composition is a drug, a compound, or a molecule (such as but not limited to an anti-sense oligonucleotide). In certain embodiments, the composition activates GABA signaling on the hepatic vagal afferent nerve. In certain embodiments, the composition increases expression of or activity of GABA transaminase (GABA T) or increases GABA production.

In certain embodiments, the composition increases or activates GABA release. In certain embodiments, the composition increases expression or activity of GABA transporters that export hepatic GABA. In certain embodiments, the composition that increases GABA release increases expression of the Solute Carrier Family 6 Member 6 (SLC6A6) gene or the Solute Carrier Family 6 Member 8 (SLC6A8) gene. In certain embodiments, the composition increases expression of or activity of TauT, a GABA transporter protein encoded by the SLC6A6 gene. In certain embodiments, the composition increases expression of or activity of creatine transporter (CRT), a GABA transporter protein encoded by SLC6A8 gene.

In certain embodiments, the composition decreases GABA re-uptake. In certain embodiments, the composition decreases expression of the Solute Carrier Family 6 Member 12 (SLC6A12) gene or the Solute Carrier Family 6 Member 13 (SLC6A13) gene. In certain embodiments, the composition decreases expression of or activity of BGT1, a GABA transporter protein encoded by the SLC6A12 gene. In certain embodiments, the composition decreases expression of or activity of GAT2, a GABA transporter protein encoded by the SLC6A13 gene.

In certain embodiments, the composition increases expression or activity of succinate semialdehyde dehydrogenase. In certain embodiments, the composition comprises an activator of sodium potassium ATPase. In certain embodiments, the composition increases hepatic mitochondrial uncoupling.

While the methods related to increasing food intake may be directed to animals, e.g., monogastric animals, the present invention is not limited to the application of said method to non-human animals. For example, there may be instances wherein the method is applied to humans in order to help increase food intake and/or gain weight.

Non-limiting examples of compounds that may be considered for weight loss and a reduction in food intake include vigabatrin and ethanolamine-O-sulfate (EOS). This finding was surprising, since those in the field believe that vigabatrin is associated with weight gain (Ben-Menachem, 2007, Epilepsia 48 Suppl 9:42-5; Lambert and Bird, 1997, Seizure 6:233-235).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1L show that GABA-Transaminase inhibition improves glucose homeostasis in obesity. HFD-induced obese mice were intraperitoneally injected with GABA-Transaminase inhibitors ethanolamine-O-sulfate (EOS) or vigabatrin (8 mg/day), or phosphate buffered saline (PBS; control) for 5 days. FIG. 1A shows body weight during treatment. FIG. 1B shows basal serum insulin (B), FIG. 1C shows glucose, and FIG. 1D shows glucose:insulin ratio pre-treatment, on treatment day 4, and after a 2-week washout. FIG. 1E shows Serum glucagon in response to EOS. FIG. 1F shows Oral glucose tolerance (OGTT), and FIG. 1G shows OGTT area under the curve (AUC) on treatment day 4. FIG. 1H shows Glucose stimulated serum insulin pre-treatment, on treatment day 4, and after a 2-week washout. FIG. 1I shows Insulin tolerance (ITT) and FIG. 1J shows ITT AUC on treatment day 4. FIG. 1L shows Tissue specific ³H-2-deoxy-D-glucose uptake during a glucose tolerance test spiked with ³H-2-deoxy-D-glucose (10 μCi/mouse) and cGMP content, indicative of vasodilatory signal (L) on treatment day 5. DPM=disintegrations per minute, NS=non-significant. ^{a,b} Bars that do not share a common letter differ significantly within injection treatment (P<0.05; number below bar denotes n per group). All data are presented as mean±SEM.

FIGS. 2A-2K show Acute hepatic GABA-Transaminase knockdown improves obesity induced metabolic dysfunction. FIG. 2A shows GABA-T mRNA expression in liver, whole brain, and pancreas after 1 week of injections with a GABA-T targeted or scramble control antisense oligonucleotide (ASO; 12.5 mg/kg IP twice weekly) in high fat diet-induced obese mice. FIG. 2B shows release of GABA (μmol/mg DNA) from hepatic slices. FIG. 2C shows body weight during treatment. FIG. 2D shows basal serum insulin. FIG. 2E shows glucose, and FIG. 2F shows glucose:insulin ratio. FIG. 2G shows oral glucose tolerance (OGTT), FIG. 2H shows OGTT area under the curve (AUC; H), FIG. 2I shows oral glucose stimulated serum insulin, FIG. 2J shows insulin tolerance (ITT), and FIG. 2K shows ITT AUC. Number below bar denotes n per group. NS=non-significant. All data are presented as mean±SEM.

FIGS. 3A-3G show one week of hepatic GABA-Transaminase knockdown improves insulin sensitivity assessed by hyperinsulinemic euglycemic clamp. High fat diet-induced obese mice received 1 week of injections with a GABA-T targeted or scramble control antisense oligonucleotide (ASO; 12.5 mg/kg IP twice weekly) before hyperinsulinemic euglycemic clamps were performed. FIG. 3A shows body weight the day of clamp procedures. FIG. 3B shows blood glucose concentrations and FIG. 3C shows glucose infusion rate during the clamps. FIG. 3D shows serum insulin concentrations before insulin infusion (basal) and during the clamp. FIG. 3E shows endogenous glucose appearance (Ra) and FIG. 3F shows glucose disappearance (Rd) before insulin infusion (basal) and during the clamp. FIG. 3G shows tissue specific ¹⁴C-2-deoxyglucose uptake (WAT=white adipose tissue, PR=perirenal adipose tissue). Number below bar denotes n per group. NS=non-significant. All data are presented as mean±SEM.

FIGS. 4A-4H show obesity induced hepatic GABA production increases phagic drive. Cumulative food intake and body mass during the first 2 weeks of GABA-T targeted or scramble control antisense oligonucleotide injections (ASO; 12.5 mg/kg IP twice weekly) in lean (FIGS. 4A, 4B) and diet-induced obese (FIGS. 4C, 4D, 4E, 4F, 4G) mice. Cumulative light cycle, dark cycle, and daily food intake (FIG. 4A) and cumulative body mass change (FIG. 4B) in lean mice. Cumulative light cycle, dark cycle, and daily food intake (FIG. 4C) and cumulative body mass change (FIG. 4D) in obese mice. Weekly cumulative food intake (FIG. 4E), cumulative body weight change (FIG. 4F), and body mass (FIGS. 4G-4H) during ASO treatment. Body mass during chronic EOS treatment (3 g/L in drinking water). All data are presented as mean±SEM.

FIGS. 5A-5F show GABA-Transaminase knockdown or inhibition decreases body mass and fat mass. Body composition in antisense oligonucleotide (ASO) treated mice was assessed by Dual-Energy X-ray Absorptiometry (DEXA) at the UC Davis Mouse Metabolic Phenotyping Center. Body composition in ethanolamine-O-sulfate (EOS) treated mice was assessed by EchoMRI 900 at The University of Arizona. FIG. 5A shows change in body mass (A), FIG. 5B shows fat mass (B), and FIG. 5C shows lean mass after 1 and 4 weeks of GABA-T targeted or scramble control ASO (12.5 mg/kg IP twice weekly) relative to pre-treatment body composition. FIG. 5D shows body mass (D), FIG. 5E shows fat mass (E), and FIG. 5F shows lean mass (F) on day 0 and 7 of EOS treatment (3 g/L in drinking water). All data are presented as mean±SEM.

FIGS. 6A-6C show associations between hepatic GABA system and glucoregulatory markers in people with obesity. Multivariate regressions including intrahepatic triglyceride % (IHTG %), hepatic ABAT (GABA-T) mRNA, and the hepatic GABA transporter (SLC6A12) mRNA as explanatory variables for variations in serum insulin (FIG. 6A) or hepatic insulin sensitivity index (HISI; FIG. 6B), ABAT and SLC6A12 mRNA (FPKMUQ; fragments per kilobase million reads upper quartile) were quantified by RNA-Seq from liver tissue. Single nucleotide polymorphisms (SNPs) in the ABAT promoter are associated with a decreased risk of type 2 diabetes (T2D; FIG. 6C). All data are presented as mean±SEM.

FIGS. 7A-7H show glucose homeostasis in obese male mice treated with the GABA-Transaminase inhibitor ethanolamine-O-sulfate (EOS; 3 g/L in drinking water). EOS effects on serum insulin (FIG. 7A), glucose (FIG. 7B), and glucose:insulin ratio (FIG. 7C) pre-treatment and after 4 days of treatment. Oral glucose tolerance (OGTT; FIG. 7D) OGTT area under the curve (OGTT AUC; 7E), and oral glucose stimulated insulin (FIG. 7F) pre-treatment and after 3 days of treatment. Insulin tolerance (ITT; FIG. 7G) and ITT AUC (FIG. 7H) pre-treatment, on day 4 of treatment (EOS), and after a 2-week washout period. ^{a,b} Bars that do not share a common letter differ significantly (P<0.05; number below bar denotes n per group). All data are presented as mean±SEM.

FIGS. 8A-8H show glucose homeostasis in lean male mice treated with GABA-Transaminase inhibitors ethanolamine-O-sulfate (EOS) or vigabatrin (8 mg/day), or phosphate buffered saline (PBS; control). Serum insulin (FIG. 8A), glucose (FIG. 8B), and glucose:insulin ratio (FIG. 8C) on treatment day 4. Oral glucose tolerance (OGTT; FIG. 8D), OGTT area under the curve (AUC; FIG. 8E), and oral glucose stimulated serum insulin (FIG. 8F) on treatment day 3. Insulin tolerance (ITT; FIG. 8G) and ITT AUC (FIG. 8H) on treatment day 4. NS=non-significant. ^{a,b} Bars that do not share a common letter differ significantly (P<0.05; number below bar denotes n per group). All data are presented as mean±SEM.

FIGS. 9A-9F show chronic hepatic GABA-Transaminase knockdown improves obesity induced metabolic dysfunction. High fat diet-induced obese mice were treated for 4 weeks with a GABA-T targeted or scramble control antisense oligonucleotide (ASO; 12.5 mg/kg IP twice weekly). Release of GABA (μmol/mg DNA) from hepatic slices (FIG. 9A). Body weight during treatment (FIG. 9B). Basal serum insulin (FIG. 9C), glucose (FIG. 9D), and glucose:insulin ratio (FIG. 9E) pre-treatment and after 4 weeks of treatment. Serum glucagon (FIG. 9F) after 4 weeks of treatment. Number below bar denotes n per group. NS=non-significant. All data are presented as mean±SEM.

FIGS. 10A-10K show glucose homeostasis in lean mice treated with the scramble control antisense oligonucleotide (ASO), or 1 of 2 GABA-Transaminase (GABA-T) targeted ASO sequences (GABA-T or GABA-T 2; 12.5 mg/kg IP twice weekly) for 4 weeks. Hepatic GABA-T mRNA expression after 1, 2, and 4 weeks of ASO injections (FIG. 10A). GABA-T mRNA expression in liver, whole-brain, and pancreas after 4 weeks of ASO injections (FIG. 10B). Body weight during treatment (FIG. 100). Basal serum insulin (FIG. 10D), glucose (FIG. 10E), and glucose:insulin ratio (FIG. 10F) pre-treatment and after 1, 2, 3, and 4 weeks of treatment. Oral glucose tolerance (OGTT; FIG. 10G), OGTT area under the curve (AUC; FIG. 10H), oral glucose stimulated serum insulin (FIG. 10I), insulin tolerance (ITT; FIG. 10J), and ITT AUC (FIG. 10K). ^{a,b,c} Bars that do not share a common letter differ significantly (P<0.05). Number below bar denotes n per group. NS=non-significant. All data are presented as mean±SEM.

FIGS. 11A-11K show GABA-Transaminase inhibition improves glucose homeostasis in sham but not vagotomy mice. HFD induced sham operated and hepatic vagotomized mice were treated with the GABA-Transaminase inhibitor ethanolamine-O-sulfate (EOS) (8 mg/day) for 5 days. Body weight during treatment (FIG. 11A). Basal serum insulin (FIG. 11B), glucose (FIG. 11C), and glucose:insulin ratio (FIG. 11D) pre-treatment, on treatment day 5, and after a 2-week washout. Oral glucose tolerance in sham mice (OGTT; FIG. 11E), oral glucose tolerance in vagotomized mice (FIG. 11F) OGTT area under the curve (AUC; FIG. 11G), and glucose stimulated serum insulin (FIG. 11H) pre-treatment, on treatment day 4, and after a 2-week washout. Insulin tolerance in sham mice (ITT; FIG. 11I) and vagotomized mice (FIG. 11J), and ITT AUC (FIG. 11K) at pre-treatment, on treatment day 5, and after a 2-week washout. NS=non-significant. ^{a,b} Bars that do not share a common letter differ significantly within injection treatment (P<0.05; number below bar denotes n per group). All data are presented as mean±SEM.

FIGS. 12A-12J show hepatic GABA-Transaminase knockdown mediated improvements in glucose homeostasis are dependent on an intact hepatic vagal nerve. Diet-induced obese hepatic vagotomized and sham operated mice were treated with a GABA-T targeted antisense oligonucleotide (ASO; 12.5 mg/kg IP twice weekly) for 4 weeks. Body weight during treatment (FIG. 12A). Basal serum insulin (FIG. 12B), glucose (FIG. 12C), and glucose:insulin ratio (FIG. 12D) pre-treatment and after 4 weeks of treatment. Serum glucagon (FIG. 12E), oral glucose tolerance (OGTT; FIG. 12F), OGTT area under the curve (AUC; FIG. 12G), oral glucose stimulated serum insulin (FIG. 12H), insulin tolerance (ITT; FIG. 12I), and ITT AUC (FIG. 12J) after 4 weeks of treatment. Number below bar denotes n per group. NS=non-significant. All data are presented as mean±SEM.

FIGS. 13A-13G show hepatic GABA-Transaminase knockdown does not affect fast induced refeeding or leptin sensitivity. Refeeding after a 16-hour fast in chow fed lean (FIG. 13A) and diet induced obese (FIG. 13B) mice after 4 weeks of GABA-T targeted or scramble control ASO injections (12.5 mg/kg IP twice weekly). Hypothalamic fasted mRNA expression of GABA-T, neuropeptide Y (NPY), agouti related peptide (AgRP), and pro-opiomelanocortin (POMC; FIG. 13C). The effect of leptin (2 mg/kg IP single injection at 6 am) on food intake (FIG. 13D) and body weight change (FIG. 13E) in obese control and GABA-T knockdown mice, and food intake (FIG. 13F) and body weight change (FIG. 13G) in lean mice. All comparisons were made within a timepoint. ^{a,b} bars that do not share a common letter differ significantly (P<0.05). NS=non-significant. All data are presented as mean±SEM.

FIGS. 14A-14I show hepatic GABA-T knockdown does not alter energy expenditure in obesity. Energy expenditure, respiratory exchange ratio, and activity level were assessed by Comprehensive Lab Animal Monitoring System (CLAMS) at the UCDavis Mouse Metabolic Phenotyping Center in diet-induced obese mice after 0, 1, and 4 weeks of GABA-T targeted or scramble control antisense oligonucleotide treatment (ASO; 12.5 mg/kg IP twice weekly). Energy expenditure during the light cycle (FIG. 14A), dark cycle (FIG. 14B), and over 24 hours (FIG. 14C). Respiratory exchange ratio (RER) during the light cycle (FIG. 14D) and dark cycle (FIG. 14E). 24 hour water intake (FIG. 14F). 24 hour activity along the horizontal X axis (XTOT; FIG. 14G), total ambulatory movement (XAMB; FIG. 14H), and vertical Z axis (ZTOT; FIG. 14I). NS=non-significant. All data are presented as mean±SEM.

FIGS. 15A-15D show hepatic vagotomy decreases light cycle food intake on HFD, while GABA-Transaminase knockdown normalizes sham mice food intake to vagotomy mice. Cumulative food intake and body weight in diet-induced obese sham operated and hepatic vagotomized mice during 1 week of baseline feeding (FIGS. 15A and 15B) and during 2 weeks of GABA-T targeted antisense oligonucleotide injections (ASO; 12.5 mg/kg IP twice weekly; FIGS. 15C-15F). Cumulative basal light cycle, dark cycle, and daily food intake (FIG. 15A) and cumulative body weight change (FIG. 15B). Cumulative ASO light cycle, dark cycle, and daily food intake (FIG. 15C) and cumulative body weight change (FIG. 15D). Weekly cumulative food intake (FIG. 15E) and cumulative body weight change (FIG. 15F). All data are presented as mean±SEM.

FIGS. 16A-16H show insulin tolerance tests (ITT) presented as raw glucose values. ITT on day 4 of EOS or Vigabatrin (8 mg/day), or PBS treatment in obese mice (FIG. 16A). ITT pre-treatment, on day 4 of oral EOS (3 g/L in drinking water) treatment, and after a 2-week washout period (FIG. 16B). ITT on day 4 of EOS or Vigabatrin (8 mg/day), or PBS treatment in lean mice (FIG. 16C). ITT in obese mice after 1 week of control or GABA-T antisense oligonucleotide (ASO) treatment (FIG. 16D). ITT in lean mice after 4 weeks of control, GABA-T, or GABA-T 2 ASO treatment (FIG. 16E). ITT in sham (FIG. 16F) and vagotomized mice (FIG. 16G) at pre-treatment, on day 5 of EOS (8 mg/day) treatment, and after a 2-week washout period. ITT in obese sham and vagotomized mice after 4 weeks of GABA-T ASO treatment (FIG. 16H). ^{a,b,c} data points that do not share a common letter differ significantly (P<0.05) within a timepoint. † Denotes the data point is not significantly different from time 0 for that group (P>0.05). Unless indicated, all other timepoints are significantly different from time 0 within a group of mice. * Denotes significance between groups specified in the panel within a timepoint. All data are presented as mean±SEM.

FIGS. 17A-17L show hepatic vagotomy protects against diet-induced hyperinsulinemia. Visual operative field for hepatic vagotomy surgeries (FIG. 17A). Arrow A indicates the hepatic branch of the vagus which was severed to vagotomize mice. Arrow A also indicates the electrode placement to record firing activity of the hepatic vagal afferent nerve (FIG. 17F). Arrow B indicates where the hepatic vagal nerve was cut after securing the electrode to eliminate vagal efferent activity (FIG. 17F). Effects of hepatic vagotomy on high fat diet (HFD) induced weight gain (FIG. 17B), serum insulin (FIG. 17C), glucose (FIG. 17D), and glucose:insulin ratio (FIG. 17E) at 0 and 9 weeks. FIGS. 17C-17E: * denotes significance (P<0.05) between bars of the same color. Regression of body weight and serum insulin concentrations during HFD feeding in sham and vagotomized mice (FIG. 17F). Effect of hepatic vagotomy after 9 weeks of HFD feeding on serum glucagon (FIG. 17G), oral glucose tolerance (OGTT; FIG. 17H), OGTT area under the curve (AUC; FIG. 17I), oral glucose stimulated serum insulin (FIG. 17J), insulin tolerance (ITT; FIG. 17K), and ITT AUC (FIG. 17L). NS=non-significant. Number below bar denotes n per group. All data are presented as mean±SEM.

FIGS. 18A-18L show acute hepatocyte depolarization depresses hepatic vagal afferent nerve activity and elevates serum insulin. Immunohistochemical validation of liver specific viral induced PSEM89S ligand gated depolarizing channel. (FIGS. 18A-18C; 10× magnification) Liver from an albumin-cre expressing mouse (FIG. 18A), and a wildtype mouse (FIG. 18B) tail-vein injected with an AAV8 encoding the PSEM89S ligand activated depolarizing channel and green fluorescent protein (GFP) whose expression is dependent on cre-recombinase. Liver from a wildtype mouse tail-vein injected with an AAV8 encoding the PSEM89S ligand activated depolarizing channel and GFP whose expression is driven by the liver specific thyroxine binding globulin (TBG) promoter (FIG. 18C). Green=GFP, blue=DAPI (nucleus), and red=background fluorescence. Hepatocyte membrane potential in lean and obese mice (FIG. 18D). FIGS. 18E, 18F, 18G, 18H, and 18I show data from albumin-cre and wildtype mice tail-vein injected with an AAV8 encoding liver specific expression of the PSEM89S ligand activated depolarizing channel whose expression is dependent on cre-recombinase. PSEM89S ligand (30 μM) induced change in hepatocyte membrane potential (FIG. 18E). PSEM89S ligand induced relative change in hepatic vagal afferent nerve activity (FIG. 18F). Data in FIG. 18F was collected concurrently with data in panel E. Serum insulin (FIG. 18G), glucose (FIG. 18H), and glucose:insulin ratio (FIG. 18I) in albumin-cre and wildtype virus injected mice 15 minutes after saline or PSEM89S ligand (30 mg/kg) administration. (FIG. 18J, 18K, 18L) Data from wildtype mice tail-vein injected with an AAV8 encoding the PSEM89S ligand activated depolarizing channel whose liver specific expression is driven by the thyroxine binding globulin (TBG) promoter. Serum insulin (FIG. 18J), glucose (FIG. 18K), and glucose:insulin ratio (FIG. 18L) in channel expressing mice injected with either saline or PSEM89S ligand (30 mg/kg) 10 minutes prior to an oral glucose load (2.5 g/kg). Alb-Cre=albumin-cre, WT=wildtype, NS=non-significant. * denotes significance (P<0.05) between groups within a time point. Number below bar denotes n per group. All data are presented as mean±SEM.

FIGS. 19A-19O show hepatic hyperpolarization protects against diet-induced metabolic dysfunction. Liver specific expression of the Kir2.1 hyperpolarizing channel in a wildtype mouse (FIG. 19A; 10× magnification). Fluorescent imaging for red=tdTomato and blue=DAPI (nucleus). Barium (BaCl; 50 μM) induced change in hepatocyte membrane potential in Kir2.1 and eGFP (control) expressing mice (FIG. 19B). Hepatic Kir2.1 expression effect on high fat diet (HFD) induced weight gain (FIG. 19C), serum insulin (FIG. 19D), glucose (FIG. 19E), and glucose:insulin ratio (FIG. 19F) at 0, 3, 6, and 9 weeks. Regression of body weight and serum insulin concentrations during HFD feeding in Kir2.1 and eGFP mice (FIG. 19G). Effect of hepatic Kir2.1 expression after 9 weeks of HFD feeding on serum glucagon FIG. 19H), oral glucose tolerance (OGTT; FIG. 19I), OGTT area under the curve (AUC; FIG. 19J), oral glucose stimulated serum insulin (FIG. 19K; * denotes significance (P<0.05) between bars of the same color), insulin tolerance (ITT; FIG. 19L), ITT AUC (FIG. 19M), pyruvate tolerance (PTT; FIG. 19N), and PTT AUC (FIG. 19O). NS=non-significant. Number below bar denotes n per group. All data are presented as mean±SEM.

FIGS. 20A-20J show hepatic slice GABA Release. Release of GABA (μmol/mg DNA) from hepatic slices (FIG. 20A), hepatic GABA-Transaminase mRNA expression (FIG. 20B), relationship between hepatic GABA release and liver triglyceride concentration (FIG. 20C), hepatic ATP concentration (nmol/g tissue; FIG. 20D), release of GABA in slices incubated with the Na+/K+ ATPase inhibitor, Ouabain (1 mM; FIG. 20E), release of GABA (μmol/mg DNA) from hepatic slices in normal (118 mM), reduced (60 mM), and low (15 mM) NaCl media (FIG. 20F), GABA media concentrations in slices treated with the BGT1 inhibitor (Betaine, 1 mM), the GAT2 inhibitor (Nipecotic Acid, NA, 1 mM), or both (FIG. 20G), reducing equivalent measures from livers of lean and obese mice (FIG. 20H), GABA media concentrations in response to inhibition of GABA-Transaminase (EOS, 5.3 mM) in liver slices (FIG. 20I), Aspartate media concentrations in lean, obese, and obese Kir2.1 expressing mice (FIG. 20J). *indicates difference from control (P<0.05). NS=non-significant. Number below bar denotes n per group. All data are presented as mean±SEM.

FIG. 21 shows the working model of hepatic lipid accumulation induced changes in hepatic metabolism and resulting changes in hepatic vagal nerve signaling to affect insulin secretion and sensitivity. High levels of β-oxidation in the obese liver increase the mitochondrial NADH₂:NAD⁺ and FADH₂:FAD⁺ ratios driving succinate to succinate semialdehyde, generating substrate for GABA-Transaminase. GABA-Transaminase produces GABA and α-ketoglutarate, a substrate for aspartate aminotransferase. Increased gluconeogenic flux in obesity drives the mitochondrial export of OAA as malate. The increased GABA release is encouraged by the depolarized membrane in obesity. GABA is co-transported with 3Na⁺ and 1Cl⁻ ions, so an increase in intracellular cation concentration (hepatocyte depolarization) encourages GABA export, while a decrease in intracellular cation concentration (hepatocyte hyperpolarization) limits GABA export. Kir2.1 expression induces hepatic K⁺ efflux and hyperpolarization, inhibiting GABA export. Obesity decreases hepatic ATP concentrations, impairing activity of the Na⁺/K⁺ ATPase pump and increasing intracellular Na⁺ concentrations, driving GABA export. This mechanism explains how hepatic lipid accumulation increases hepatic GABA release. Abbreviations: OAA=oxaloacetate, AST=aspartate aminotransferase, GABA-T=GABA-Transaminase, α-KG=α-ketoglutarate, SSADH=succinate semialdehyde dehydrogenase.

FIGS. 22A-22C show associations between hepatic GABA system and glucoregulatory markers in obese humans. Multivariate regressions including intrahepatic triglyceride % (IHTG %) and the mRNA for the hepatic GABA transporters (Slc6A6, Slc6A8, Slc6A12, and Scl6A12) as explanatory variables for variations in glucose infusion rate during a hyperinsulinemic euglycemic clamp (μMol/kg fat free mass/min; FIG. 22A), and the glucose disposal rate calculated during a hyperinsulinemic-euglycemic clamp (Glucose Rd, % increase; FIG. 22B). mRNA (FPKM; Fragments Per Kilobase of transcript per Million mapped reads) was quantified by RNA-Seq from liver tissue. (FIG. 22C) Single nucleotide polymorphisms (SNPs) that cause missense mutations in Slc6A12 or Slc6A13 are associated with an increased incidence of type 2 diabetes (T2D) adjusted for body mass index (BMI). Regression data are presented as mean±SEM.

FIGS. 23A-23J show hepatic Kir2.1 expression alters glucose homeostasis in the lean mouse. Hepatic Kir2.1 expression effects on serum insulin (FIG. 23A) glucose (FIG. 23B), glucose:insulin ratio (FIG. 23C), oral glucose tolerance (OGTT; FIG. 23D), OGTT area under the curve (AUC; FIG. 23E), oral glucose stimulated serum insulin (FIG. 23F; * denotes significance (P<0.05) between bars of the same color), insulin tolerance (ITT; FIG. 23G) ITT AUC (FIG. 23H), pyruvate tolerance (PTT; FIG. 23I), and PTT AUC (FIG. 23J). NS=non-significant. Number below bar denotes n per group. All data are presented as mean±SEM.

FIGS. 24A-24E show glucose homeostasis in Kir2.1 and eGFP control mice at 3 weeks of high fat diet feeding. Effect of hepatic Kir2.1 expression on oral glucose tolerance (OGTT; FIG. 24A), OGTT area under the curve (AUC; FIG. 24B), oral glucose stimulated serum insulin (FIG. 24C), insulin tolerance (ITT; FIG. 24D), and ITT AUC (FIG. 24E). NS=non-significant. Number below bar denotes n per group. All data are presented as mean±SEM.

FIGS. 25A-25D show insulin tolerance tests (ITT) presented as raw glucose values. ITT in HFD fed sham and vagotomized mice (FIG. 25A). ITT in Kir2.1 and eGFP control mice on chow diet (FIG. 25B), and after 3 (FIG. 25C), and 9 weeks of HFD feeding (FIG. 25D). t Denotes the data point is not significantly different from time 0 for that group (P>0.05). Unless indicated, all other timepoints are significantly different from time 0 within a group of mice. * Denotes significance between groups specified in the panel within a timepoint. All data are presented as mean±SEM.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise. Suitable methods and materials for the practice and/or testing of embodiments of the disclosure are described below. Such methods and materials are illustrative only and are not intended to be limiting. Other methods and materials similar or equivalent to those described herein can be used. All publications, patent applications, patents, and other references mentioned herein are incorporated by reference in their entirety for all purposes. In case of conflict, the present specification, including explanations of terms, will control. Although methods and materials similar or equivalent to those described herein can be used to practice or test the disclosed technology, suitable methods and materials are described below. The materials, methods, and examples are illustrative only and not intended to be limiting. In order to facilitate review of the various embodiments of the disclosure, the following explanations of specific terms are provided.

Animal: As used herein, the term “animal” includes but is not limited to a human, mouse, rat, rabbit, dog, cat, pig, chicken, non-human primates, etc.

Antisense oligonucleotide: As used herein, the term “antisense oligonucleotide” refers to a single-stranded oligonucleotide having a nucleobase sequence that permits hybridization to a corresponding region or segment of a target nucleic acid. Antisense technology is emerging as an effective means for reducing the expression of specific gene products.

Effective Amount. The term “effective amount,” as used herein, refers to a dosage of a compound or a composition effective for eliciting a desired effect. This term as used herein may also refer to an amount effective at bringing about a desired in vivo effect in an animal, mammal, human, etc. Effective amount may vary depending upon body mass of the individual to be treated, the health and physical condition of the individual to be treated, the taxonomic group of individual to be treated, the formulation of the composition, depending on the evaluation, and other relevant factors of a medical condition of an individual varies between individuals obtain.

Treatment: As used herein, the terms “treat” or “treatment” or “treating” refers to both therapeutic treatment and prophylactic or preventative measures, wherein the object is to prevent or slow the development of a disease or condition, such as slow down the development of obesity, or reducing at least one adverse effect or symptom of a condition, disease or disorder, e.g., any disorder characterized by insufficient or undesired function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a condition, as well as those likely to develop a condition due to genetic susceptibility or other factors such as weight, diet and health.

### DETAILED DESCRIPTION OF THE INVENTION

The present invention features compositions and methods for altering hepatic GABA reuptake and/or release to treat obesity and obesity-related conditions, including hyperphagia, hypertension, insulin resistance, and hyperinsulinemia.

The methods and compositions (e.g., compounds, drugs, molecules, e.g., siRNA, etc.) herein may be used for treating obesity and obesity related complications such as but not limited to insulin resistance, diabetes (e.g., type II diabetes), and hypertension. For example, the present invention features methods for treating obesity-related complications using compositions (e.g., compounds, drugs, molecules, e.g., siRNA, etc.) that inhibit the activity or expression of (or silence) GABA-transaminase, hepatic succinate semialdehyde dehydrogenase, TauT (protein encoded for by SLC6A6), CRT (protein encoded for by SLC6A8), the like, or a combination thereof. Alternatively, they may increase BGT1 (protein encoded for by SLC6A12) and/or GAT2 (protein encoded for by SLC6A13) to encourage GABA re-uptake.

The present invention also features methods for treating obesity-related complications by hyperpolarizing liver cells or by preventing obesity induced depolarization of liver cells. This changing membrane potential will alter the activity of the GABA transporters. In some embodiments, the compositions of the present invention improve insulin sensitivity and glucose clearance, decrease blood glucose and insulin concentrations, and/or decrease/normalize blood pressure.

In some embodiments, the present invention is for inhibiting hepatic GABA release; increasing hepatic aspartate release; hyperpolarizing the hepatocyte/preventing the obesity induced depolarization of the hepatocyte and GABA transporter mediated release, while encouraging GABA transporter mediated GABA re-uptake; preventing GABA signaling on the hepatic vagal afferent nerve; increasing Aspartate signaling on the hepatic vagal afferent nerve; blocking muscarinic 3 receptor signaling on the beta and alpha cell; blocking pancreatic parasympathetic efferent signaling; increasing muscarinic receptor signaling on endothelial cells in the vasculature to limit vasoconstriction/encourage vasodilation; enhancing skeletal muscle parasympathetic efferent signaling; and the like.

As previously discussed, the present invention features methods of treating obesity or an obesity-related condition in a subject in need thereof. In certain embodiments, the method comprises administering to the subject a therapeutic amount of a composition for increasing hepatic GABA re-uptake and/or decreasing hepatic GABA release, wherein increasing hepatic GABA re-uptake or decreasing hepatic GABA release decreases blood glucose and improves insulin sensitivity. In certain embodiments, the composition prevents obesity-induced depolarization of hepatocytes. In certain embodiments, the composition normalizes blood pressure. In certain embodiments, the composition reduces hepatic mitochondrial uncoupling.

In certain embodiments, the composition comprises an inhibitor of GABA-T. In certain embodiments, the composition comprises an activator of BGT1. In certain embodiments, the composition comprises an activator of GAT2. In certain embodiments, the composition comprises an inhibitor of M3R for inhibiting insulin release. In certain embodiments, the composition comprises an activator of M3R for improving insulin sensitivity and stimulating insulin release. In certain embodiments, the composition comprises an inhibitor of UCP2. In certain embodiments, the composition comprises an inhibitor of hepatic succinate semialdehyde dehydrogenase. In some embodiments, the composition comprises an inhibitor of GHB production. In some embodiments, the composition comprises an inhibitor of GHB conversion to succinate semialdehyde (SSA). In some embodiments, the composition comprises a GHB dehydrogenase inhibitor.

In other embodiments, the composition is a drug, a compound, or a molecule. In certain embodiments, the molecule is an anti-sense oligonucleotide. In certain embodiments, the composition inhibits GABA signaling on the hepatic vagal afferent nerve.

In certain embodiments, the obesity-related condition is diabetes, hyperglycemia, insulin resistance, glucose intolerance, or hypertension.

In certain embodiments, the composition causes a fasting blood glucose of 120 mg/dL or less. In certain embodiments, the composition causes a fasting blood glucose of 110 mg/dL or less. In certain embodiments, the composition causes a fasting blood glucose of 100 mg/dL or less. In certain embodiments, the composition causes a fasting blood glucose of 90 mg/dL or less. In certain embodiments, the composition causes a fasting blood glucose from 90 mg/dL to 100 mg/dL. In certain embodiments, the composition causes a fasting insulin level of 5 mmol/mL or less. In certain embodiments, the composition causes a fasting insulin level of 10 mmol/mL or less. In certain embodiments, the composition causes a fasting insulin level from 2 to 10 mmol/mL.

In certain embodiments, the composition comprises ethanolamine-O-sulfate (EOS). In certain embodiments, the composition comprises vigabatrin. In certain embodiments, the composition does not cross the blood-brain barrier. In certain embodiments, the composition comprises a derivative of vigabatrin or EOS that does not cross the blood-brain barrier.

The present invention also features methods for improving insulin sensitivity in a subject in need thereof. In certain embodiments, the method comprises administering to the subject a therapeutic amount of a composition for increasing hepatic GABA reuptake or decreasing hepatic GABA release, wherein increasing hepatic GABA reuptake or decreasing hepatic GABA release improves insulin sensitivity. In certain embodiments, the composition restores insulin sensitivity to that of a non-diabetic individual.

The present invention also features methods for improving insulin sensitivity and limiting hyperinsulinemia in a subject in need thereof. In some embodiments, the method comprises administering to the subject a therapeutic amount of a composition for decreasing hepatic GABA synthesis or hepatic GABA release, wherein decreasing hepatic GABA synthesis or release improves insulin sensitivity and decreases hyperinsulinemia.

In some embodiments, the obesity-related condition is diabetes, hyperglycemia, insulin resistance, glucose intolerance, excess body adiposity, excessive food intake, or hypertension. The present invention also features a pharmaceutical composition for treating an obesity-related condition, wherein the composition is effective to decrease blood glucose, decrease blood insulin, improve insulin sensitivity, increase glucose tolerance, and decrease/normalize blood pressure or a combination thereof.

In certain embodiments, the composition comprises an inhibitor of beta-cell M3R for inhibiting insulin release. In certain embodiments, the composition comprises an activator of endothelial cell M3R for improving insulin sensitivity and stimulating insulin release. In certain embodiments, the composition comprises an inhibitor of UCP2. In certain embodiments, the composition comprises an inhibitor of hepatic succinate semialdehyde dehydrogenase. In some embodiments, the composition comprises an inhibitor of GHB production. In some embodiments, the composition comprises an inhibitor of GHB conversion to succinate semialdehyde (SSA). In some embodiments, the composition comprises a GHB dehydrogenase inhibitor.

In certain embodiments, the composition comprises ethanolamine-O-sulfate (EOS). In certain embodiments, the composition comprises vigabatrin. In certain embodiments, the composition does not cross the blood-brain barrier. In certain embodiments, the composition comprises a derivative of vigabatrin or EOS that does not cross the blood-brain barrier.

In other embodiments, the hepatic GABA transporters are electrogenic and comprising members of the Na⁺/Cl⁻-dependent neurotransmitter transporter (SLC6) family, wherein the members comprise proteins encoded for by Slc6A12 (Betaine GABA transporter 1, BGT1), Slc6A13 (GABA transporter 2, GAT2), Slc6A6 (Taurine Transporter, TauT), and Slc6A8 (Creatine transporter, CRT). BGT1 and GAT2 both co-transport 3 Na⁺, 1 Cl⁻ and GABA, moving 2 positive charges in the direction of GABA transport.

In some embodiments, the GABA transporter moves a novel hepatokine, GABA, that is dysregulated in obesity and whose release can be manipulated to mute or exacerbate the glucoregulatory dysfunction common to obesity.

In other embodiments, wherein the composition comprises an inhibitor of GABA-T or ABAT gene expression, an activator of BGT1 activity and/or expression or an activator of SLC6A12 expression, an activator of GAT2 activity and/or expression or an activator of SLC6A13 expression, an inhibitor of TauT activity and/or expression or an inhibitor of SLC6A6 expression, an inhibitor of CRT activity and/or expression or an inhibitor of SLC6A8 expression, an inhibitor of Beta-cell M3R for inhibiting insulin release or an activator of endothelial cell M3R to improve insulin sensitivity or Beta-cell M3R to stimulate insulin release.

### EXAMPLES

The following are non-limiting examples enabling the present invention. It is to be understood that said examples are not intended to limit the present invention in any way. Equivalents or substitutes are within the scope of the present invention.

### Example 1: A Critical Role of Hepatic GABA in the Metabolic Dysfunction and Hyperphagia of Obesity

All animal studies were conducted using male wildtype C57BL/6J mice purchased from Jackson Laboratories or bred in-house (Bar Harbor, Me.). Mice were kept on a 14-hour light/10-hour dark schedule and housed 3-5 mice per cage until 1 week prior to study initiation, at which point animals were individually housed. Studies were conducted in lean chow fed mice (7013 NIH-31, Teklad WI, 3.1 kcal/g, 18% kcal from fat, 59% kcal from carbohydrate, 23% kcal from protein) at 12-16 weeks of age. Studies in diet-induced obese mice dosed intraperitoneally with GABA transaminase inhibitors, mice treated with ethanolamine-O-sulfate in their drinking water, and mice treated with GABA-T targeted or scramble control antisense oligonucleotides (ASO) were performed after 8-10 weeks on a high fat diet (TD 06414, Teklad WI, 5.1 kcal/g, 60.3% kcal from fat, 21.3% kcal from carbohydrate, 18.4% kcal from protein; 20-26 weeks of age). For ASO studies, mice were stratified by body weight and assigned to an injection treatment (control or GABA-T). Studies in obese vagotomy and sham mice were performed after 9 weeks of high fat diet feeding. Unless fasted, mice had ad libitum access to food and water. All studies were approved by The University of Arizona Institutional Animal Care and Use Committee.

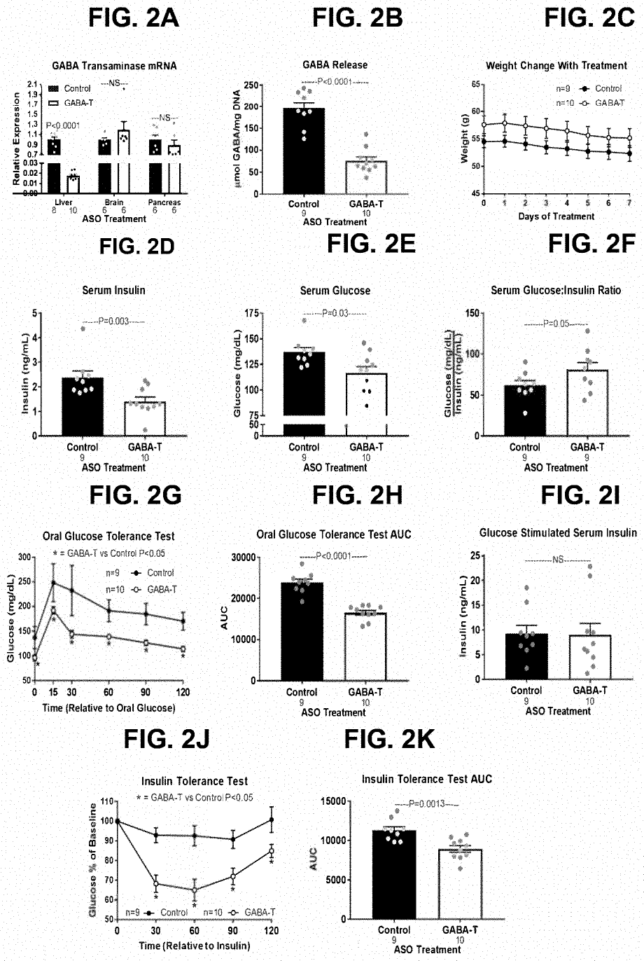

Wildtype lean or diet-induced obese mice received twice-weekly intraperitoneal injections (12.5 mg/kg; 0.1 mL/10 g body weight) of murine GABA-Transaminase (GABA-T) targeted antisense oligonucleotides (ASO; IONIS 1160575) or scramble control ASO (IONIS 549144) for 1 or 4 weeks prior to experimentation. The control ASO does not have complementarity to known genes and was employed to demonstrate the specificity of target reduction. 2′,4′-constrained 2′-O-ethyl (cEt) ASOs were synthesized at Ionis Pharmaceuticals (Carlsbad, Calif.) as described previously. Treatment with this GABA-T targeted ASO reduces hepatic GABA-T mRNA by 98% in adult mice within 1 week (FIG. 2A). For studies after 1 week of ASO injections, an OGTT (day 8) and ITT (day 10) were performed and mice were sacrificed to assess liver slice GABA release (day 12). For studies after 4 weeks of ASO injections, an OGTT (day 29) and ITT (day 30) were performed and completed all additional studies within 1 week while continuing biweekly ASO injections before mice were sacrificed to assess liver slice GABA release.

Surgeries were performed in 12-week old male C57BL/6J mice under isoflurane anesthesia. Mice were randomly assigned to a surgical group (sham or vagotomy). A ventral midline incision through the skin and peritoneum allowed us to isolate the hepatic vagus nerve as it branched from the esophagus. In vagotomized mice, the hepatic vagal nerve was severed, while it remained intact in sham operated mice. The peritoneum was sutured with absorbable polyglactin 910 suture and the skin with nylon suture. Mice were given a single post-operative dose of slow release formulated buprenorphine analgesic (1.2 mg/kg slow release, sub-cutaneous). Food intake and body weight were monitored daily and sutures were removed 7 days post-operation.

Wildtype lean and obese mice were randomly divided into treatment groups and dosed daily with 8 mg of ethanolamine-O-sulfate (EOS; Sigma-Aldrich, St. Louis, Mo.), vigabatrin (United States Pharmacopeia, Rockville, Md.) or PBS. Obese sham and vagotomy mice were dosed daily with 8 mg of EOS. In all cases, basal bleeds were taken prior to initiation of an ITT. Lean mice received treatment by oral gavage (0.3 mL/mouse) while obese mice were treated by intraperitoneal injection (0.3 mL/mouse). Pre-treatment studies were conducted in the week immediately prior to beginning drug administration. Daily doses took place at 9 am each day and oral glucose tolerance tests (OGTT) and insulin tolerance tests (ITT) were performed on the third and fourth days of treatment in lean mice, respectively. In wildtype obese mice, OGTT and ITT were performed on the fourth day of treatment in separate cohorts. On the fifth day of treatment, 2DG clearance studies were performed. In sham/vagotomy mice, OGTT and ITT were performed on the fourth and fifth day of treatment, respectively. After a 2-week washout period without treatment injection, a basal bleed and bleed 15 minutes after an oral glucose gavage (2.5 mg/kg) to determine oral glucose stimulated insulin secretion were performed.

In a separate set of studies, an ITT was performed in obese mice to establish insulin resistance. Subsequently, EOS was provided ad libitum in the water (3 g/L) for 4 days. An OGTT and ITT were performed on days 3 and 4 of treatment, respectively. The water was then removed, and an ITT was performed 2 weeks later to establish the timing of restoration of insulin resistance after drug removal.

We measured food weight and body weight at 6 am and 6 pm for the first 14 days of control or GABA-T ASO injections in lean and obese wildtype mice and GABA-T ASO injections in sham and vagotomy mice. Weeks 3 and 4 food and body weight measurements were taken at 6 pm on day 21 and 28 of ASO injections.

Mice were housed on wood chip bedding (Harlan Laboratories; Cat #7090 Sani-Chips) to limit consumption of nutrients from bedding during the fasting period. Mice were fasted for 16 hours beginning at 5 μm and food was returned at 9 am. Food and body weight were measured at 10 am, 11 am, and 1 pm to determine 1, 2, and 3-4 hour fast-induced refeeding.

Lean mice or diet-induced obese control or GABA-T targeted ASO treated mice received an intraperitoneal injection of phosphate buffered saline (PBS; 0.1 mL/10 g body weight) on day 1 and leptin (2 mg/kg; 0.1 mL/10 g body weight; CAT#498-OB, R&D Systems, Minneapolis, Minn.) on day 2 at 6 am. Mice were not fasted before injections. Food and body weight were measured every day at 6 am and 6 pm.

Energy expenditure studies and body composition measurements were performed by the UC Davis Mouse Metabolic Phenotyping Center. Twelve male DIO (C57BL/6J, strain 380050) mice were sent to UC Davis from the Renquist Lab at ˜20 weeks of age and were acclimated for 2 weeks on investigator provided diet (Research diets D12492). Mice were injected IP with control or ABAT ASO (12.5 mg/kg) twice a week for 4 weeks (8 injections total). Energy expenditure and physical activity were evaluated in 6 saline and 6 ABAT ASO treated mice by indirect calorimetry in the CLAMS (Comprehensive Lab Animal Monitoring System, Columbus Instruments) system three times at baseline (−3), 7, and 28 days post first ASO injection. Body composition was assessed under isoflurane anesthesia by DEXA after each CLAMS run. Animals were allowed to recover from anesthesia before returning to their home cage except for the final (3rd) DEXA run, after which animals were terminated and no tissues collected. Animals were acclimated to the CLAMS cages for 48 hours and to the light and temperature-controlled chamber for 24 hours prior to testing. Animals were held and calorimetry data was collected for 48 hrs. Analyzed data constitutes data collected from 48 hours of continuous measurement (2 light/2 dark cycles). Oxygen consumption and carbon dioxide production were measured and used to calculate energy expenditure (or heat production, kilocalories (kcal)) and respiratory exchange ratio (RER: VCO2/VO2). Cage-mounted sensors detected and recorded measurements of physical activity, food intake and water intake. Body composition was measured by dual-energy X-ray absorptiometry under isoflurane anesthesia, using a Lunar PIXImus II Densitometer (GE Medical Systems, Chalfont St. Giles, UK) immediately after completion of the indirect respiration calorimetry measurements.

Body composition was assessed in diet-induced obese mice on day 0 and 7 days after continuous provision of EOS in the drinking water (3 g/L) using an EchoMRI 900 with A10 insert for mice. Calibration was performed daily and had the water stage set to on.

Clamps were performed as previously described 46. Briefly, one week prior to the experiment, diet-induced obese mice underwent surgical catheterization of the jugular vein under isoflurane anesthesia. Mice were given a single post-operative dose of slow release formulated buprenorphine analgesic (1.2 mg/kg slow release, sub-cutaneous) and food and body weight were assessed daily for 1 week. The same day following surgery completion mice received their first injection of either the scramble control or GABA-T targeted ASO (12.5 mg/kg; 0.1 mL/10 g body weight). Four days post-surgery mice received a second ASO injection, and clamps were performed 7 days after catheterization. Following a 5-h fast (starting at 0800h), clamps were performed using unrestrained, conscious mice. The clamp procedure consisted of a 90-min tracer equilibration period (−90 to 0 min), followed by a 120-min clamp period (0 to 120 min). To begin the equilibration period, mice were infused with 3-[3H]-D-glucose (0.05 μCi/μl in saline) at a rate of 10 μL/min for 2 minutes and then decreased to a rate of 1 μL/min for the remaining 90 minutes. All blood was collected from the tip of the tail in heparinized capillary tubes and immediately spun down to collect plasma for tracer and insulin analysis. All timepoints during the clamp are relative to the start of insulin infusion at time 0. Blood for tracer and insulin analysis was taken at −10 and 0 minutes. At 0 minutes mice were infused with donor blood (5 μL/min continuously throughout study), insulin (4 mU/kg/min; pump rate 1 μL/min continuously throughout study), and a variable infusion of 3-[3H]-D-glucose (0.05 μCi/μl in 50% dextrose) to maintain euglycemia. Glucose was assessed from whole-blood directly from the tail tip by glucometer (Manufacture #D2ASCCONKIT, Bayer, Leverkusen, Germany) every 10 minutes for 120 minutes starting at time 0. The glucose infusion rate was adjusted to maintain a blood glucose concentration in the range of 100-120 mg/dL. Blood for tracer measurements was taken at 80, 90, 100, and 120 minutes, and blood for insulin was taken at 100 and 120 minutes. To estimate insulin-stimulated glucose fluxes in tissues, mice were given a bolus of 14C-2-deoxyglucose (12 μCi in 48 μL followed by a 100 μL saline flush) after the 120 min timepoint. Blood samples were collected 2, 10, and 25 minutes following the bolus. Mice were anesthetized with isoflurane using the bell-jar method and the soleus, quadricep, calf, white adipose tissue, and perirenal adipose tissue were collected and immediately frozen in liquid nitrogen for analysis of tissue specific glucose uptake. Plasma samples were deproteinized using barium hydroxide and zinc sulfate and tracer analysis and tracer standards was processed as described 46. Plasma glucose was analyzed by colorimetric assay (Cat. #G7519, Pointe Scientific Inc., Canton Mich.) from the samples collected for tracer measurement. Plasma insulin was analyzed by enzyme-linked immunosorbent assay (ELISA; Cat. #80-INSMSU-E01, E10, Alpco, Salem, N.H.).

Oral glucose (2.5 g/kg; 0.1 mL/10 g body weight; Chem-Impex Int'l Inc., Wood Dale, Ill.) was given to 4 hour fasted individually housed mice. All glucose tolerance tests began at 1 μm and glucose was measured in whole blood, collected from the tail vein, by glucometer (Manufacture #D2ASCCONKIT, Bayer, Leverkusen, Germany) at 0, 15, 30, 60, 90, and 120 minutes after glucose gavage. Blood for serum insulin (oral glucose stimulated insulin secretion; OGSIS) and glucose determination was collected from the tail vein 15 minutes following glucose administration.

Intraperitoneal insulin (0.5 U/kg; 0.1 mL/10 g body weight; Sigma Aldrich, St. Louis, Mo.) was given to 4 hour fasted individually housed mice. All insulin tolerance tests began at 1 μm and glucose was measured in whole blood, collected from the tail vein, by glucometer (Manufacture #D2ASCCONKIT, Bayer, Leverkusen, Germany) at 0, 30, 60, 90, and 120 minutes after insulin injection.

Liver slices from ASO treated mice were incubated ex vivo to measure hepatic GABA release. A peristaltic pump perfusion system was used to deliver warmed KH buffer to the liver through the portal vein. Briefly, mice were anesthetized with an intraperitoneal injection of ketamine (10 mg/mL) and diazepam (0.5 mg/mL). Once mice were unresponsive, an incision in the lower abdomen through the skin and peritoneal membrane was made vertically through the chest along with transverse incisions on both sides to expose the liver. A 30-gauge needle was inserted into the hepatoportal vein to blanch the liver. The inferior vena cava was cut to relieve pressure in the circulatory system and allow blood to drain. The perfusion continued for several minutes at a rate of 4 mL/minute until the liver was completely blanched. The liver was removed and washed in warm PBS before being sliced into 0.2 mm slices using a Thomas Sadie-Riggs Tissue Slicer. Two liver slices were taken from each mouse. Tissue slices were placed individually into a well on a 12-well plate pre-filled with 1 mL of KH buffer that had been sitting in an incubator set to 37° C. and gassed with 5% CO2. Liver slices were incubated in the initial well for 1 hour to stabilize before being transferred to a fresh well pre-filled with KH buffer. After 1 hour in the second well, tissue and media were collected. Liver slice samples and KH media samples from both wells of each mouse were pooled. Liver slices were snap frozen in liquid nitrogen, while media was centrifuged for 5 minutes at 10,000×g at 4° C. to remove tissue debris and both were frozen and stored at −80° C. pending analysis.

For all liver slice GABA release data, the media was thawed collected from the ex vivo hepatic slice culture on ice. GABA was then measured in the supernatant using a commercially available ELISA (REF #BA E-2500, Labor Diagnostika Nord, Nordhorn, Germany). μmol GABA concentrations were corrected for liver slice DNA concentrations.

On the fifth day of EOS or PBS treatment 3H-2-deoxy-D-glucose (2DG; 10 uCi/mouse; PerkinElmer, Waltham, Mass.) was given to 4-hour fasted individually housed mice. Studies were performed in 2 cohorts on 2 different days. 2DG was given by oral gavage in a solution of glucose (2.5 g/kg) and each mouse received the same dose based off the average body weight for their cohort (0.1 mL/10 g body weight). Mice were anesthetized by isoflurane and sacrificed by cervical dislocation 45 minutes following oral gavage. Liver, soleus, quadriceps femoris, and gonadal white adipose tissue were collected, weighed, and dissolved overnight in 1N NaOH (0.5 mL/50 mg tissue) at 55° C. on a shaker plate. 0.5 mL of dissolved tissue was added to 5 mL of scintillation cocktail (Ultima Gold, PerkinElmer, Waltham, Mass.) and disintegrations per minute (DPM) were measured using a LS 6500 Multipurpose Scintillation Counter (Beckman Coulter, Brea, Calif.). DPM/g tissue weight was determined for each tissue and normalized based on a correction factor calculated by the average total DPM/g for all tissues divided by the total DPM/g for all tissues of the individual mouse.

On the fifth day of EOS or PBS treatment, mice were sacrificed and the quadricep and soleus tissues were collected and frozen on dry ice. Prior to analysis, frozen quadricep were powdered using a liquid nitrogen cooled mortar and pestle to obtain homogenous muscle samples. 15-20 mg of quadricep and the entire soleus tissue (6-12 mg) were homogenized in 200 μL of a 5% trichloroacetic acid solution. Following 15 minutes of centrifugation at 3,000×g at 4° C., supernatant was transferred to a fresh tube for analysis of muscle cGMP by enzyme-linked immunosorbent assay (ELISA; ADI-900-164, Enzo Life Sciences, Farmingdale, N.Y.).

Prior to analysis, frozen livers were powdered using a liquid nitrogen cooled mortar and pestle to obtain homogenous liver samples. To measure liver DNA content (ng dsDNA/g tissue), 10-20 mg of powdered liver was sonicated in 500 μL DEPC H2O and dsDNA determined by fluorometric assay (Cat. #P7589, Invitrogen, Waltham, Mass.). Whole liver and hypothalamic mRNA were isolated from powered liver samples with TRI Reagent® (Life Technologies, Grand Island, N.Y.) and phenol contamination was eliminated by using water-saturated butanol and ether as previously described 47. cDNA was synthesized by reverse transcription with Verso cDNA synthesis kit (Thermo Scientific, Inc., Waltham, Mass.), and qPCR performed using SYBR 2× mastermix (Bio-Rad Laboratories, Hercules, Calif.) and the Biorad iQTM5 iCycler (Bio-Rad Laboratories, Hercules, Calif.). Expression of GABA-Transaminase (ABAT), β-actin (ACTβ), insulin (Ins), neuropeptide Y (NPY), agouti related peptide (AgRP), and pro-opiomelanocortin (POMC) mRNA were measured using primers as previously described (Ramakers et al., 2003). LinReg PCR analysis software was used to determine the efficiency of amplification from raw output data. ACTβ served as the reference gene for liver and brain tissue, and Ins served as the reference gene for pancreas tissue for calculating fold change in ABAT gene expression using the efficiency-ΔΔCt method.

Within 2 hours of collection, blood was left to clot at room temperature for 20 minutes. Thereafter, the blood was centrifuged at 3,000×g for 30 minutes at 4° C. and serum was collected. Serum was stored at −80° C. until metabolite and hormone analyses. A colorimetric assay was used to analyze serum glucose (Cat. #G7519, Pointe Scientific Inc., Canton Mich.). Serum insulin was analyzed by enzyme-linked immunosorbent assay (ELISA; Cat. #80-INSMSU-E01,E10, Alpco, Salem, N.H.). Serum glucagon was analyzed by enzyme-linked immunosorbent assay (ELISA; Cat. #10-1281-01, Mercodia, Uppsala, Sweden) from tail vein blood collected at 9 am from fed mice.

A total of 19 men and women with obesity who were scheduled for bariatric surgery at Barnes-Jewish Hospital in St. Louis, Mo. participated in this study, which was conducted at Washington University School of Medicine in St. Louis. Subjects provided written, informed consent before participating in this study, which was approved by the Human Research Protection Office at Washington University School of Medicine in St. Louis, Mo. (ClinicalTrials.gov NCT00981500). Intrahepatic triglyceride content was determined by using magnetic resonance imaging (3.0-T superconducting magnet; Siemens, Iselin, N.J.) in the Center for Clinical Imaging Research. A 7-hour (3.5-h basal period and 3.5-h insulin infusion period) HECP, in conjunction with stable isotopically labelled glucose tracer infusion, was then conducted in the Clinical Translational Research Unit (CTRU), as previously described. This procedure was performed to determine: i) hepatic insulin sensitivity, which was assessed as the product of the basal endogenous glucose production rate (in μmol·kg fat-free mass (FFM)-1·min-1) and fasting plasma insulin concentration (in mU/L).

Liver tissue was obtained by needle biopsy during the bariatric surgical procedure, before any intra-operative procedures were performed. Liver tissue was rinsed in sterile saline, immediately frozen in liquid nitrogen, then stored at −80° C. until RNA extraction. Total RNA was isolated from frozen hepatic tissue samples by using Trizol reagent (Invitrogen, Carlsbad, Calif.). Library preparation was performed with total RNA and cDNA fragments were sequenced on an Illumina HiSeq-4000. The fragments per kilobase million reads upper quartile (FPKM-UQ) values were calculated and used for further gene expression analyses. All RNA-seq data used in this study have been deposited into the NCBI GEO database under accession number GSE144414.

The data were analyzed in SAS Enterprise Guide 7.1 (SAS Inst., Cary, N.C.), using a mixed-model ANOVA for all analyses except the multivariate regression analyses performed on human clinical data. ANOVA tests do not have a one-tailed vs. two-tailed option, because the distributions they are based on have only one tail. When comparisons between all means were required, a Tukey's adjustment was used for multiple comparisons. When comparisons of means were limited (e.g. only within a timepoint or treatment), a Bonferonni correction was used for multiple comparisons. For the analysis of ITT and OGTT, repeated measures ANOVA were performed by including time point in the analysis. Analyses were conducted separately for chow and HFD fed mice. For the studies using the GABA-T inhibitors the main effect was treatment (PBS, Vigabatrin, or EOS). For EOS treated sham and vagotomy mice the main effects were surgery (sham or vagotomy) and treatment (PBS or EOS). Pre-, during, and post-treatment measures were taken for basal glucose, insulin, and glucose stimulated insulin, thus a repeated measure analysis including time (pre-, during, or post-treatment) was performed separately within each injection or surgical treatment. For analysis of the effect of EOS on 2DG uptake and cGMP, analysis was performed separately for each tissue. For ASO studies the main effect was injection treatment (control of GABA-T ASO). For the vagotomy analyses the main effect was surgery (sham or vagotomy). Pre-treatment and ASO week 4 measures were taken for basal glucose, insulin, and the glucose:insulin ratio, thus a repeated measure analysis including time (pre-, or week 4-treatment) was performed separately within each injection or surgical treatment. A multivariate regression analysis was performed on data from human clinical patients using IHTG %, ABAT mRNA, and SLC6a12 mRNA as explanatory variables for variations in serum insulin, HOMA-IR, M-Value, and Glucose Rd. Statistics performed by the UC Davis Mouse Metabolic Phenotyping Center are described as follows: data are presented as means±SEM and a Student t-test was used to test for significant differences between groups. Multiple linear regression analysis (analysis of covariance, ANCOVA) was used to assess the impact of covariates (e.g. body weight or lean mass) on energy expenditure. The EE ANCOVA analysis done for this work was provided by the NIDDK Mouse Metabolic Phenotyping Centers (MMPC, www.mmpc.org) using their Energy Expenditure Analysis page (http://www.mmpc.org/shared/regression.aspx) and supported by grant DK076169. All insulin tolerance tests are presented as a percentage of baseline glucose and additionally presented as raw glucose values. Human data was analyzed using a multivariate regression including IHTG content, and ABAT, SLC6A6, SLC6A8, SLC6A12, and SLC6A13 mRNA expression as independent variables with Type 3 test of fixed effects used to determine significance and estimates derived from maximum likelihood estimation. All graphs were generated using GraphPad Prism 8 (GraphPad Software Inc., La Jolla, Calif.).

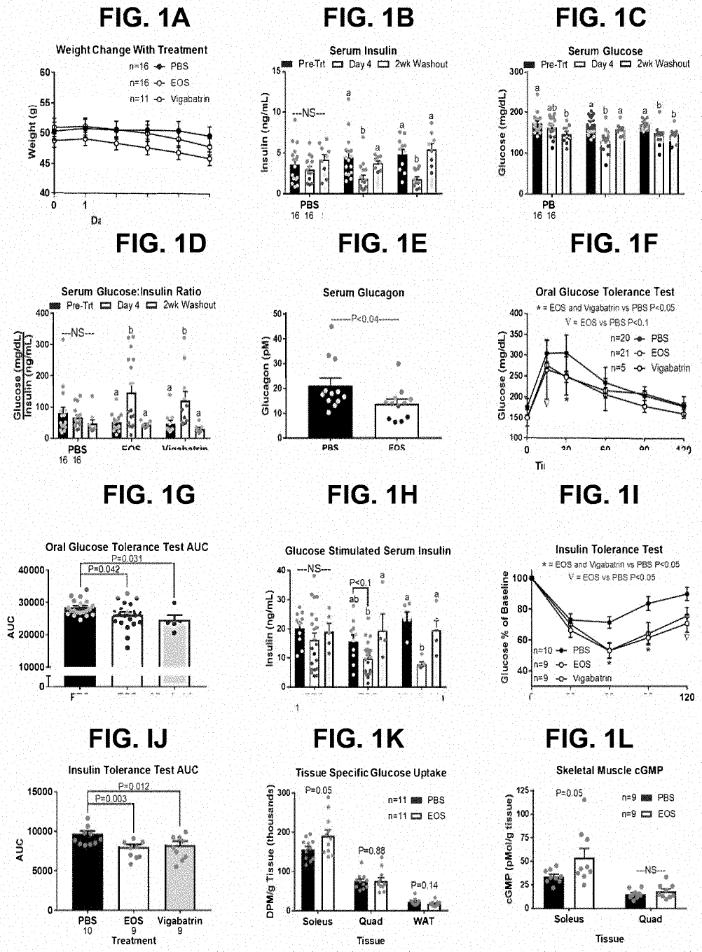

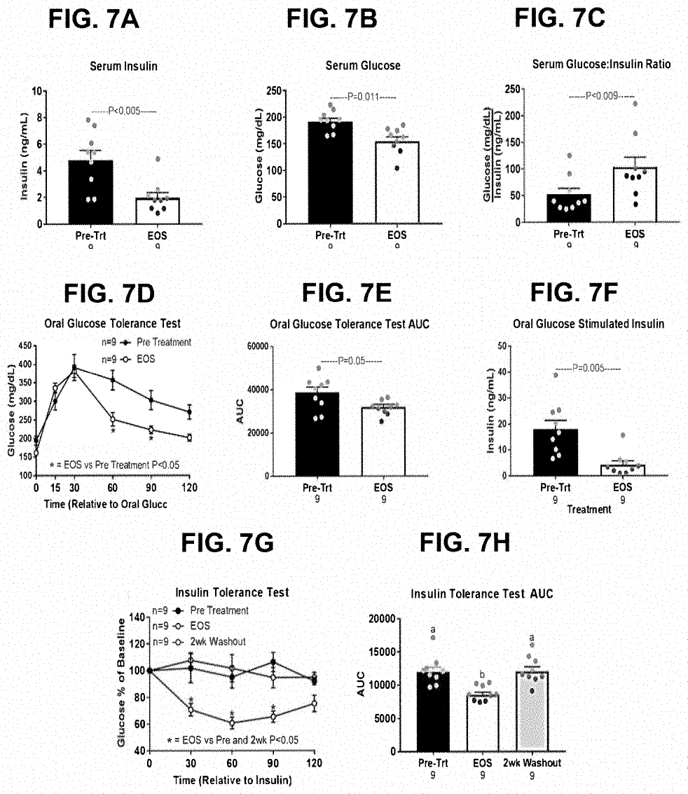

To directly assess the effect of GABA-T in obesity-induced metabolic dysfunction high fat diet-induced obese mice were treated with one of two irreversible GABA-T inhibitors, ethanolamine-0-sulphate (EOS) or vigabatrin (8 mg/day). Both reduce hepatic GABA-T activity by over 90% within two days. Through 5 days of treatment, body weight remained similar among EOS, vigabatrin, and saline injected mice (FIG. 1A). Four days of EOS or vigabatrin treatment decreased serum insulin and glucose concentrations and increased the glucose:insulin ratio relative to pre-treatment (FIGS. 1B-1D). Two-weeks washout from EOS or vigabatrin resulted in a return of serum insulin and the glucose:insulin ratio to pre-treatment levels (FIGS. 1B-1D). EOS treatment (5 days) decreased serum glucagon relative to control mice (FIG. 1E). Glucose clearance during an oral glucose tolerance test (OGTT) was improved by 4 days of GABA-T inhibition (FIGS. 1F-1G). Coincident with this improved clearance, glucose stimulated serum insulin was decreased by vigabatrin and tended to be decreased by EOS relative to pre-treatment concentrations (FIG. 1H). Two-weeks washout from EOS and vigabatrin markedly increased glucose stimulated serum insulin (FIG. 1H). Both GABA-T inhibitors improved insulin sensitivity assessed by insulin tolerance test (ITT) within 4 days of initiating treatment (FIG. 1I-1J). As oral drug delivery is preferred in clinic, another series of studies was conducted with EOS provided in the drinking water (3 g/L) for 4 days. EOS in the drinking water similarly improved measures of glucose homeostasis and the response washed out after 2 weeks without EOS in the drinking water (FIG. 7A-7H).

To further assess the mechanism by which GABA-T inhibition improves glucose clearance, tissue specific ³H-2-deoxy-D-glucose (2DG) uptake was measured following an oral glucose gavage on day 5 of EOS or saline treatment. EOS treatment increased 2DG uptake by the soleus (22%) but did not affect 2DG clearance by the quadriceps femoris (quad) or gonadal white adipose tissue (WAT; FIG. 1K). Given that blood perfusion is a key regulator of insulin action and glucose clearance, cGMP was subsequently measured. cGMP is a key second messenger downstream of nitric oxide (NO) signaling that regulates blood flow. EOS increased cGMP in the soleus (59%) but had no effect in quad (FIG. 1L).

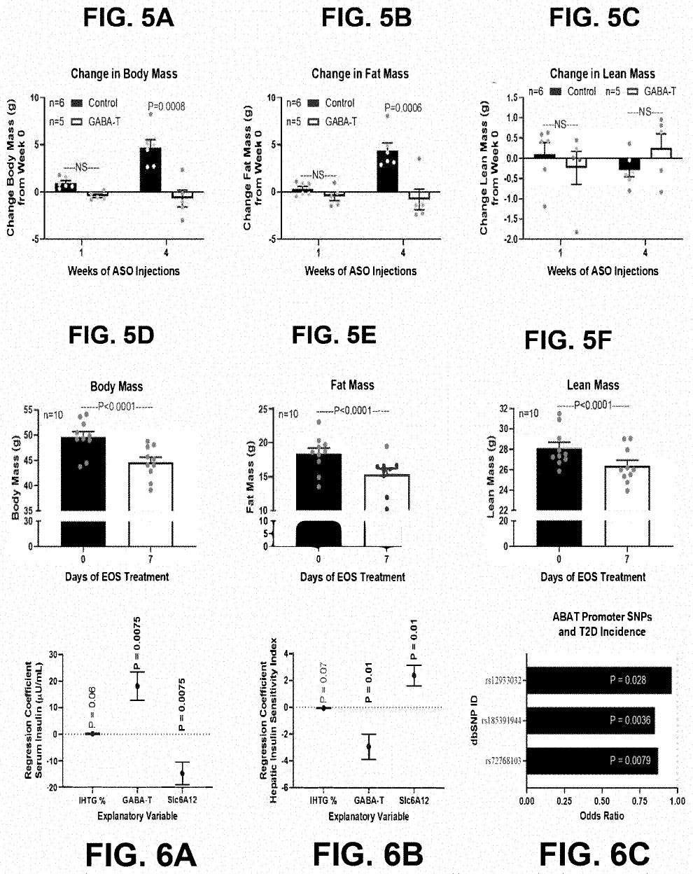

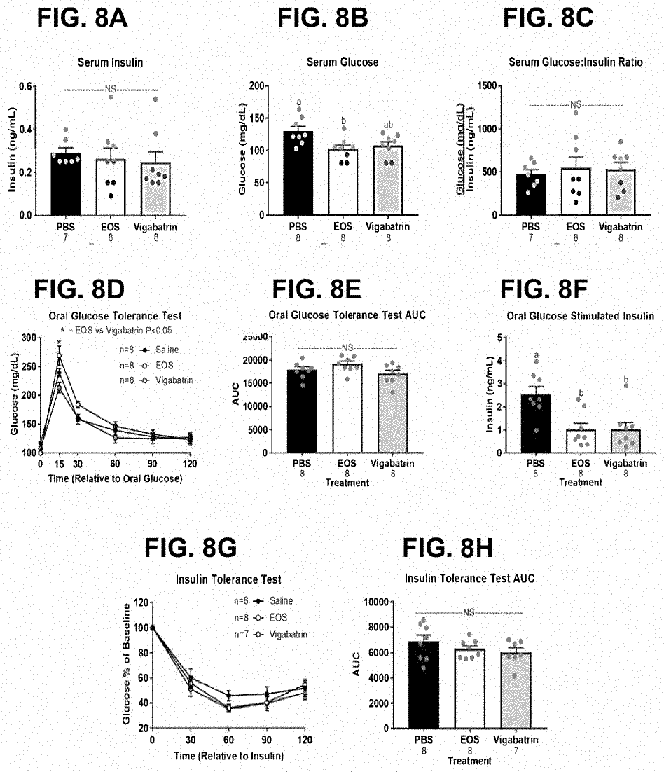

In lean mice, which have low hepatic GABA production, there was no effect of EOS or vigabatrin (8 mg/day) on serum insulin concentrations, glucose tolerance, or insulin sensitivity (FIGS. 8A-8H. In lean mice, GABA-T inhibition decreased glucose stimulated serum insulin, while EOS decreased serum glucose (FIG. 8A, 8F). Without wishing to limit the present invention to any particular theory or mechanism, it is believed that GABA-T inhibition decreases serum glucose by directly impairing hepatic gluconeogenic flux from TCA cycle intermediates (FIG. 5A-5F).

To overcome the limitation of global pharmacologic inhibitors, an ASO model was next used to specifically knockdown hepatic GABA-T expression. Peripherally administered ASOs do not cross the blood brain barrier. Outside the central nervous system, the liver and pancreas express the most GABA-T. A GABA-T targeted ASO (12.5 mg/kg IP twice weekly) decreased hepatic GABA-T mRNA expression by >98% within 1 week. Importantly, this GABA-T targeted ASO did not affect pancreatic or whole-brain GABA-T mRNA expression (FIG. 2A). To establish the key role of GABA-T in liver slice GABA production, ex vivo liver slice GABA release was measured. GABA-T knockdown cut obesity induced liver slice GABA release by 61% (FIG. 2B). One week of GABA-T knockdown in obese mice did not affect body weight but decreased basal serum insulin and glucose concentrations and elevated the glucose:insulin ratio (FIGS. 2C-2F). GABA-T targeted ASO injections also improved oral glucose clearance without affecting oral glucose stimulated serum insulin concentrations (FIGS. 2G-2I), and improved insulin sensitivity compared to scramble control ASO injected mice (FIGS. 2J-2K). These findings directly implicate hepatic GABA production in the development of obesity-induced hyperinsulinemia and insulin resistance.

Compared with control ASO, chronic (4 week) GABA-T ASO treatment decreased ex vivo liver slice GABA release, and decreased body mass and serum insulin, while increasing the glucose:insulin ratio, and (FIGS. 8A-8C, 8E). Thus, the metabolic response to GABA-T knockdown persists. Admittedly, a decrease in body mass with chronic ASO treatment could contribute to the improvements in glucose homeostasis.

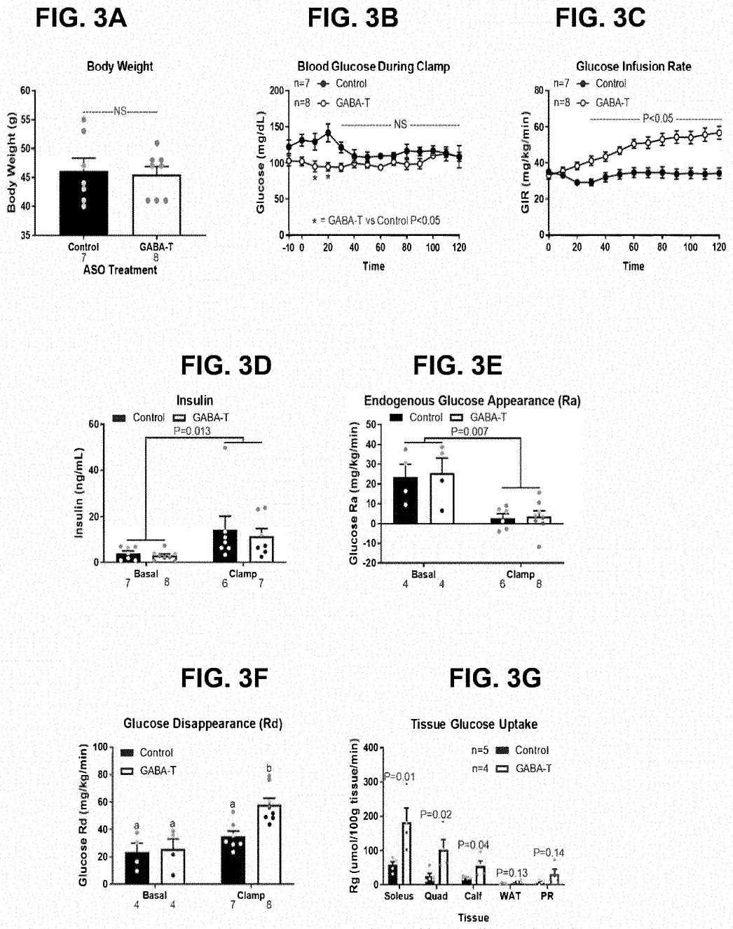

To more precisely assess the effect of GABA-T knockdown on insulin action before body mass is affected, hyperinsulinemic-euglycemic clamps were performed in diet-induced obese mice treated with scramble control or GABA-T targeted ASOs for 1 week. Body weight on the day of clamp was not affected by treatment (FIG. 3A). All timepoints are relative to the onset of insulin (4 mU/kg/min) and variable rate 3-[³H]-D-glucose in dextrose infusion at time 0, with the basal period referencing pre-0 measurements. Blood glucose concentrations were lower in GABA-T ASO treated mice during the first 20 minutes of the clamp, but not different between ASO treatments from 30-120 minutes of the clamp during which euglycemia was achieved in both groups (FIG. 3B). To maintain the same level of euglycemia, GABA-T knockdown mice required a higher glucose infusion rate (GIR: mg/kg/min) which was significant from 30-120 minutes of the clamp (FIG. 3C). Plasma insulin concentrations did not differ by ASO treatment during either the basal period or during the clamp (FIG. 3D). Importantly, insulin concentrations were elevated during the clamp (FIG. 3D; P=0.013). GABA-T knockdown did not affect the rate of endogenous glucose appearance (Ra) during the basal or clamp periods (FIG. 3E). The basal rate of glucose disappearance (Rd) did not differ between ASO treatments. However, hyperinsulinemia during the clamp nearly doubled Rd in GABA-T knockdown but not control mice (FIG. 3F). After 120 minutes of the clamp, mice received a bolus of ¹⁴C-2-dexyglucose and were sacrificed 30 minutes later to assess tissue specific glucose uptake. GABA-T knockdown improved glucose uptake by the soleus, quadricep, and calf skeletal muscles (FIG. 3G).

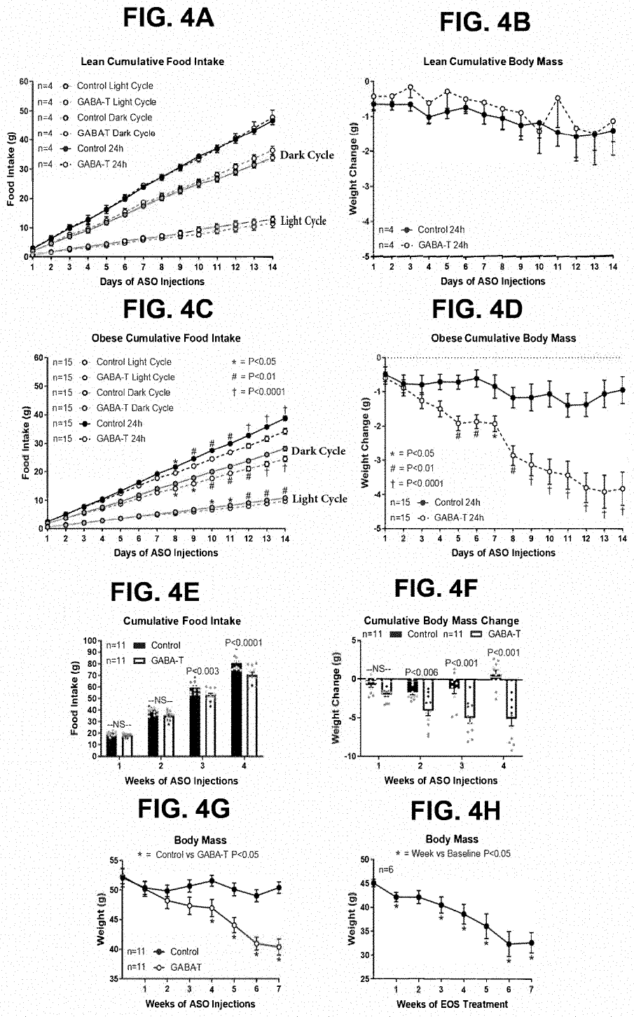

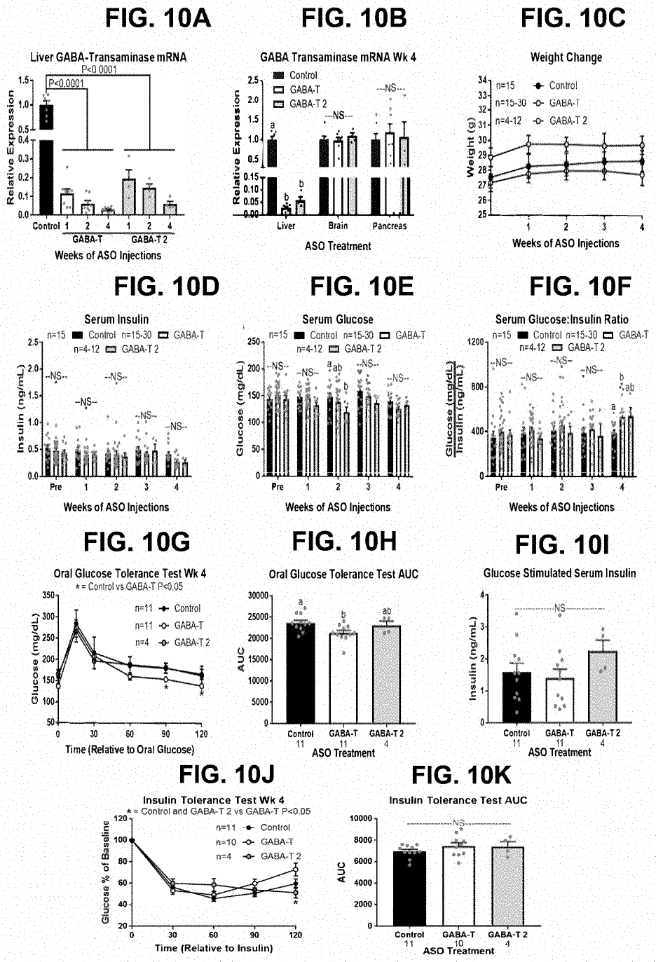

In chow fed mice hepatic GABA production is limited. In turn, it is expected to observe minimal responses to inhibition of the GABA shunt in lean mice. Two GABA-T targeted ASOs were tested that both decreased hepatic GABA-T mRNA expression by 80% within 1 week and 94% after 4 weeks of treatment. Neither altered pancreatic or whole-brain GABA-T mRNA expression (FIGS. 10A, 10B). Chronic (4 week) GABA-T knockdown did not affect body weight or glucoregulatory measures in lean mice (FIGS. 10C-10K, Extended Data FIG. 4C-4K). This agrees with observations that acute GABA-T inhibition with EOS does not alter glucose homeostasis in lean mice (FIGS. 8A-8H). Instead, beneficial effects of GABA-T inhibition are specific to obesity, due to an increase in the GABA shunt activity and subsequent hepatic GABA production observed during obesity.

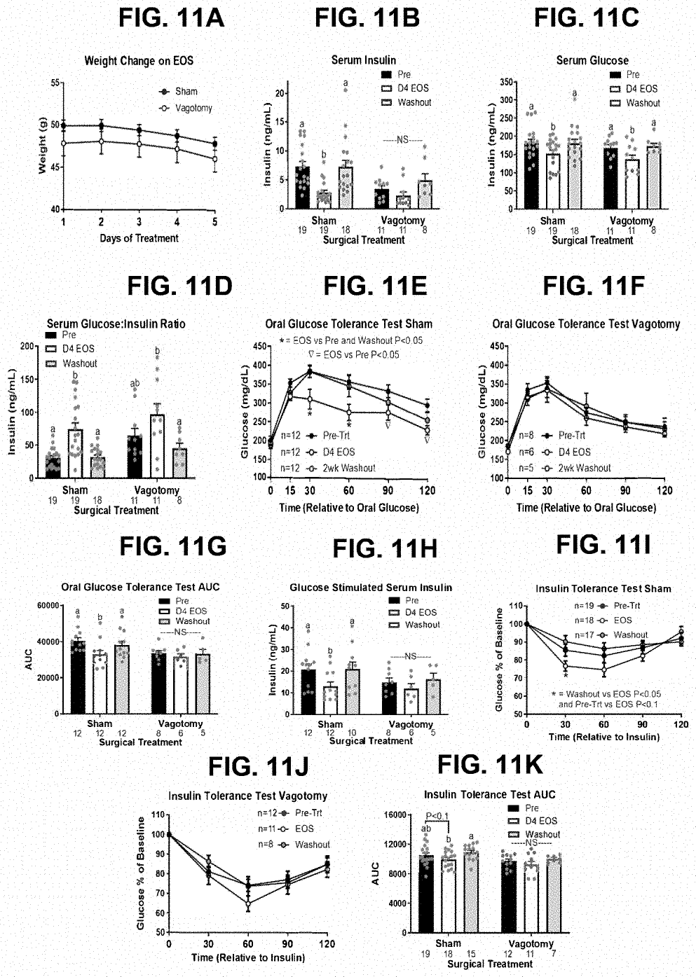

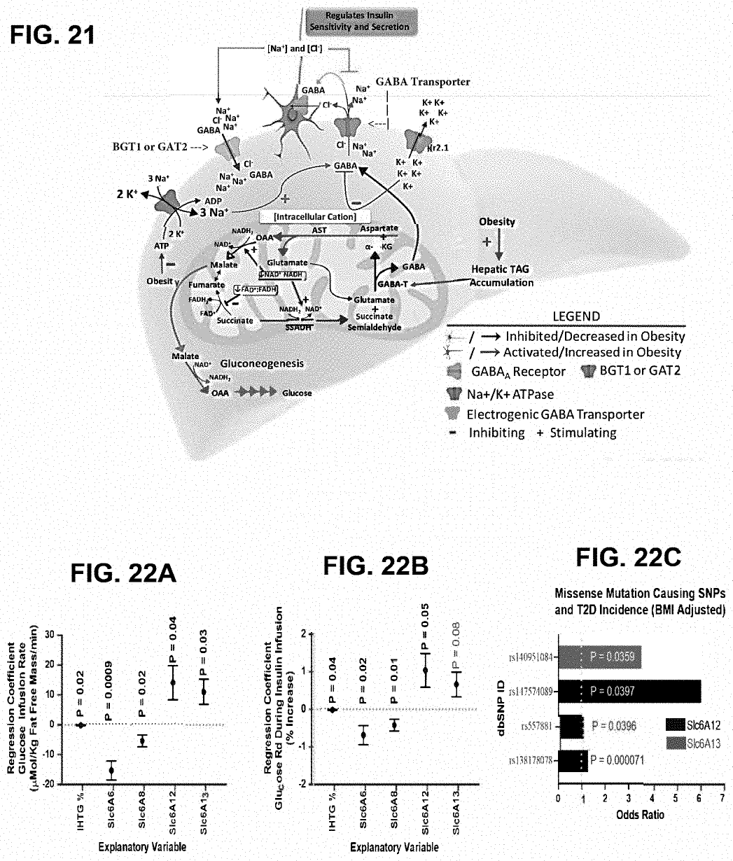

Hepatic vagotomy prevents the liver from altering afferent signaling to the brain, without affecting normal vagal afferent input originating from the nodose. In turn, vagotomy prevents inhibition of the vagal afferent tone by GABA produced in the liver. Without wishing to limit the present invention to any particular theory or mechanism, it is believed that hepatic GABA mediates the effects that are reported herein by acting on the HVAN to induce hyperinsulinemia and insulin resistance. Accordingly, the effect of EOS treatment was assessed in HFD-induced obese hepatic vagotomized and sham operated mice. Although body weight did not differ between surgical groups during EOS treatment (FIG. 11A), the response to EOS was dependent on an intact hepatic vagal nerve. In sham operated mice EOS decreased serum insulin and glucose, elevated the glucose:insulin ratio, improved oral glucose tolerance, diminished glucose stimulated serum insulin concentrations, and tended to improve insulin sensitivity as measured by an insulin tolerance test (FIGS. 11B-11K). Washout restored all parameters to pre-treatment measures. Vagotomy eliminated most of the responses to EOS (FIGS. 11B-11K). Since hepatic GABA production supports gluconeogenic flux (FIG. 21), it was expected GABA-T inhibition to decrease gluconeogenesis through direct actions at the liver. In turn, diminished hepatic glucose output explains the vagal nerve independent decrease in serum glucose during EOS treatment in hepatic vagotomized mice (FIG. 11C).

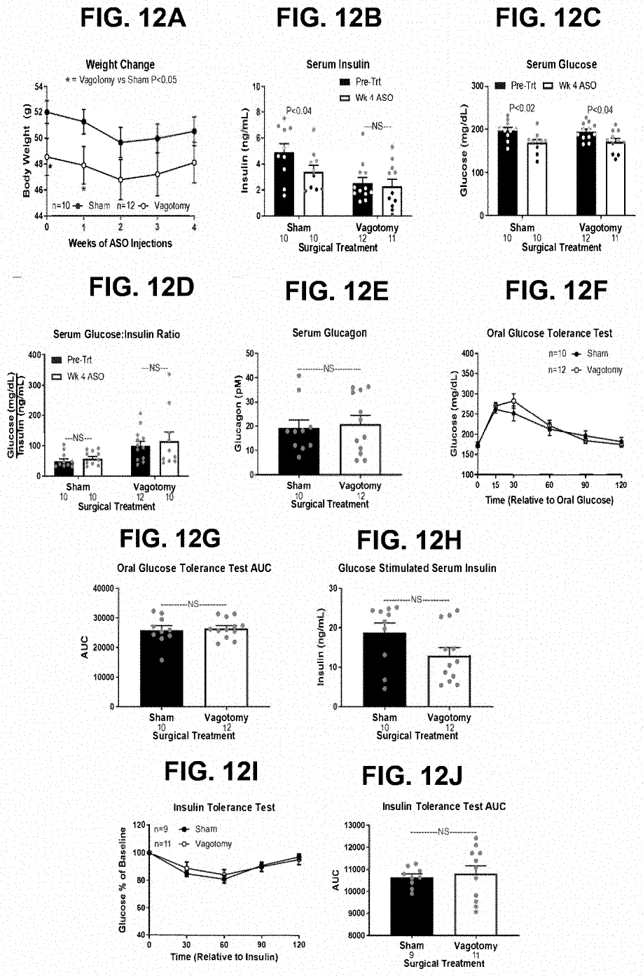

The effect of 4 weeks of GABA-T knockdown (12.5 mg/kg GABA-T targeted ASO IP twice weekly) was assessed in diet-induced obese sham operated and hepatic vagotomized mice. Consistent with previous observations, pre-treatment body weight was lower in obese, vagotomized mice compared to sham operated control mice (FIG. 12A). As shown with pharmacological GABA-T inhibition (EOS), GABA-T knockdown decreased basal serum glucose concentrations independent of the hepatic vagal nerve (FIG. 12C), again likely due to reduced hepatic glucose output. GABA-T knockdown decreased basal serum insulin, improved oral glucose tolerance, limited oral glucose stimulated serum insulin, and improved insulin sensitivity in sham, but not in vagotomized mice (FIGS. 12B, 12E-12J). Hepatic vagotomy protects against obesity-induced insulin resistance GABA-T knockdown allowed sham operated animals to achieve similar glucose tolerance, glucose stimulated serum insulin, and insulin sensitivity to that measured in hepatic vagotomized mice.

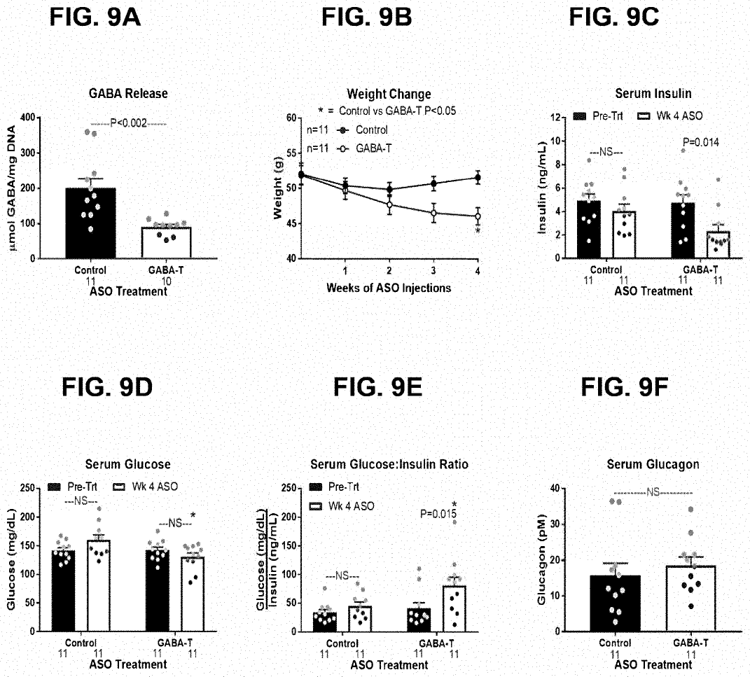

Given that hepatic GABA-T knockdown for 4 weeks decreased body weight in obese mice (FIG. 9B), it is hypothesized that GABA-T knockdown may decrease food intake or increase energy expenditure. Accordingly, daily food intake and body mass were assessed during the light and dark cycle for the first 2 weeks of ASO treatment in lean and obese mice. In lean mice, GABA-T knockdown did not affect cumulative light cycle, dark cycle, or daily food intake (FIG. 4A). Cumulative daily body mass change was also not affected by GABA-T knockdown in lean mice (FIG. 4B). In obese mice, GABA-T knockdown decreased cumulative light cycle, dark cycle, and daily food intake (FIG. 4C). Cumulative weekly food intake remained lower through 4 weeks of GABA-T knockdown (FIG. 4E). This suppression of food intake was accompanied by a decrease in body mass in GABA-T ASO treated mice (FIGS. 4D and 4F). In fact, GABA-T knockdown continued to decrease body weight during 7 weeks of treatment (FIG. 4G). Similarly, 7 weeks of continuous exposure to EOS in the drinking water (3 g/L) dramatically decreased body mass in diet-induced obese mice (FIG. 4H). These data suggest that sensitivity to the weight loss effect of limiting hepatocyte GABA production is maintained throughout treatment but is less significant as body mass returns to normal.

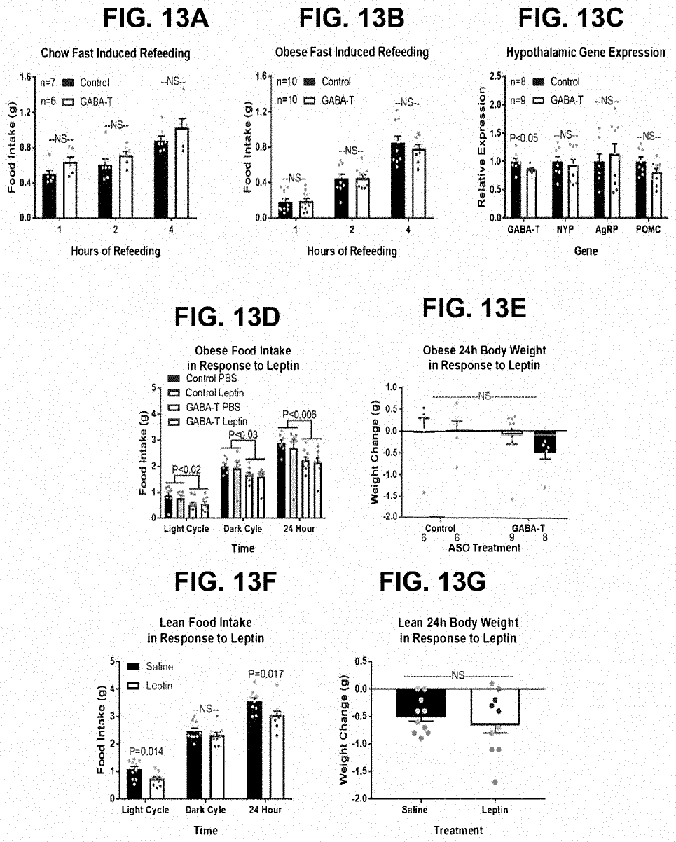

Interestingly, GABA-T knockdown did not alter food intake in response to a 16-hour fast in either lean or obese mice (FIGS. 13A-13B). This proposes that hepatic GABA signals to regulate ad libitum light and dark cycle food intake without affecting the fasting-induced drive for refeeding. Accordingly, GABA-T knockdown did not affect fasting mRNA expression of the canonical hypothalamic neuropeptides regulating food intake (NPY, AgRP, and POMC; FIG. 13C). It cannot be ruled out an effect of central GABA-T knockdown as a 14% reduction was observed in hypothalamic GABA-T expression at 7 weeks of GABA-T ASO injections (FIG. 13C). The anorexigenic hormone leptin induces satiety and weight loss, while leptin resistance in obesity contributes to hyperphagia and weight gain. GABA-T knockdown in obesity may have improved leptin sensitivity as a potential was tested as a mechanism to decrease appetite and cause weight loss. A single 6AM injection of leptin (2 mg/kg) did not affect food intake in mice on either ASO treatment at any timepoint, suggesting that mice on both treatments remained leptin resistant (FIG. 13D). Consistent with the decreased food intake previously observed in response to GABA-T knockdown, GABA-T knockdown decreased light cycle, dark cycle, and 24-hour food intake independent of injection (FIG. 13D). Relative weight change was also not affected by leptin in control or GABA-T ASO obese mice (FIG. 13E). Thus, enhanced leptin sensitivity is not mediating the diminished phagic drive in response to GABA-T knockdown in obesity. A single 6AM leptin injection decreased light cycle and 24-hour food intake in chow fed mice without affecting body weight (FIGS. 13F-13G).

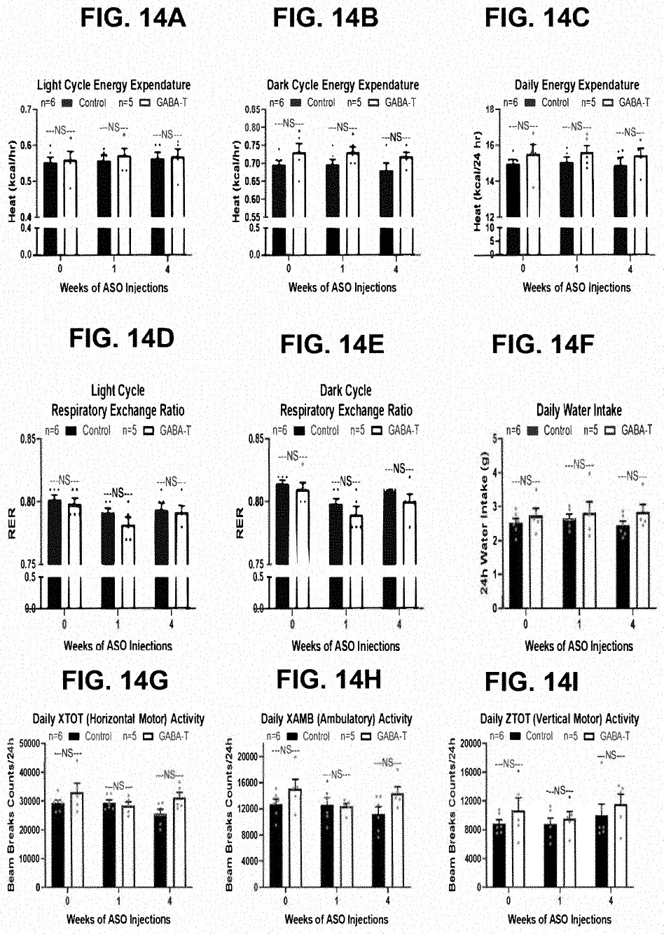

It was next investigated whether altered energy expenditure contributed to the body mass loss with GABA-T knockdown in obesity. Energy expenditure (kcal/hr), assessed by respiration calorimetry and corrected using the covariate of lean body mass, was not altered by 1 or 4 weeks of GABA-T knockdown (FIGS. 14A-14C). In addition, there was no effect of GABA-T knockdown on the composition of oxidized macronutrients (respiratory exchange ratio; RER), daily water intake, or daily activity (FIGS. 14D-14I). Thus, the effects of GABA-T knockdown on body mass appear independent of energy expenditure or nutrient oxidation.

Body composition, assessed by Dual-Energy X-ray Absorptiometry (DEXA), revealed that 4 weeks of GABA-T knockdown decreased total body mass and fat mass without affecting lean mass (FIGS. 5A-5C). The decreased fat mass with GABA-T knockdown suggests that weight loss induced by limiting hepatocyte GABA production reflects a loss of adiposity. Body composition was additionally assessed on day 0 and 7 of providing obese mice with EOS in their drinking water (3 g/L). It was found that EOS treatment decreased total body mass (10%), fat mass (16.27%), and lean mass (6.15%). Although there was a small loss of lean mass, diminished fat mass contributes the majority of lost body mass with EOS treatment.

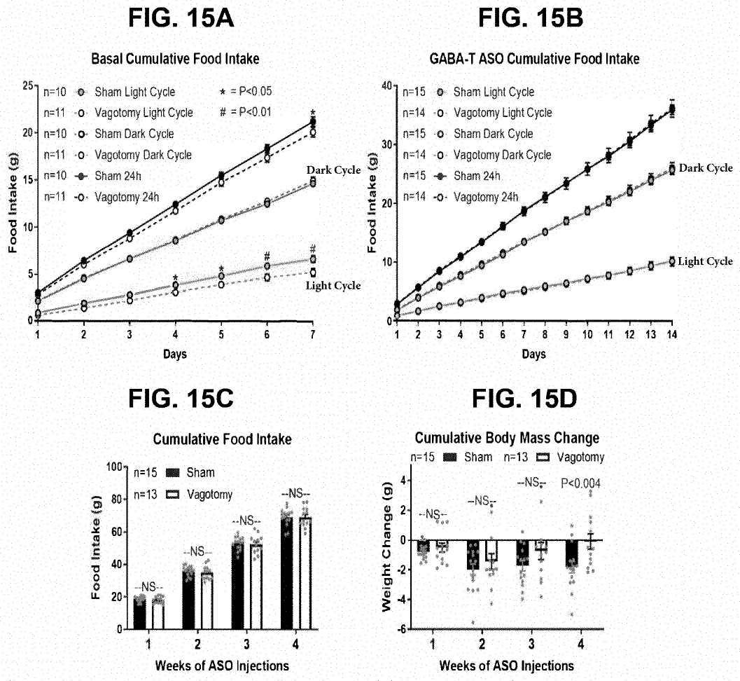

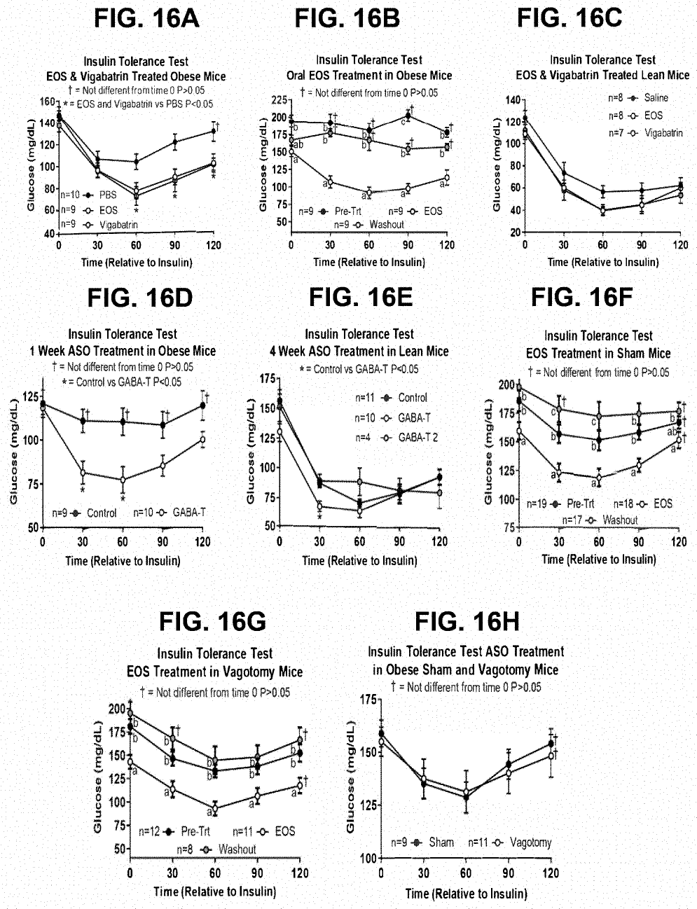

Hepatic vagotomy decreased weight gain on a high fat diet. Herein, hepatic vagotomy decreases 1-week cumulative light cycle food intake by 22% in diet-induced obese mice, resulting in a 5.3% decrease in cumulative 24-hour food intake (FIG. 15A). Daily food intake measurements were continued for the next 2 weeks as all mice were treated with the GABA-T targeted ASO. In response to GABA-T knockdown, the previously observed difference in food intake in sham operated and vagotomized mice was eliminated. In fact, cumulative light cycle, dark cycle, and daily food intake were similar in sham operated and vagotomized mice (FIG. 15B). The GABA-T ASO resulted in a greater cumulative week 4 body mass loss in sham operated than in vagotomized mice which experienced no net change in body mass (FIG. 15D). Thus, the glucoregulatory, phagic, and body mass changes in response to GABA-T knockdown all appear to be dependent on the hepatic vagal afferent nerve. These data support that hepatic vagotomy eliminates the GABA-induced decrease in HVAN activity from being communicated to the brain and accordingly protects against obesity-induced perturbations of glucose and energy homeostasis.

To understand the potential clinical relevance of these findings, the hepatic mRNA expression of ABAT (encodes for GABA-T) and GABA transporter (SLC6A6, encodes for taurine transporter, TauT); SLC6A8; encodes for the creatine transporter, CRT; SLC6A12; encodes for the Betaine-GABA Transporter 1, BGT1, and SLC6A13; encodes for GABA Transporter 2, GAT2) was assessed in 19 people with obesity (age 45±3 yrs old, 2 men and 17 women) who were carefully characterized by measuring intrahepatic triglyceride (IHTG) content using magnetic resonance imaging (MRI) and insulin sensitivity using the hyperinsulinemic-euglycemic clamp procedure (HECP) in conjunction with stable isotopically labeled glucose tracer infusion). The subjects had a wide range in IHTG content, plasma insulin concentration and measures of insulin sensitivity (Data Table 1). There was a trend toward a positive correlation between IHTG content and basal plasma insulin concentration (P=0.06) and a negative correlation between IHTG content and hepatic insulin sensitivity index (HISI, a product of the basal endogenous glucose production rate and plasma insulin concentration, P=0.07; Data Table 2).

DATA TABLE 1
Metabolic characteristics of the study subjects (n = 19).

| | Mean ± SEM | Range |
|---|---|---|
| Body mass index (kg/m²) | 45.1 ± 1.3 | 35.9-55.6 |
| Intrahepatic | 11.4 ± 1.9 | 2.7-28.0 |
| triglyceride content (%) | | |
| Glucose (mg/dL) | 97 ± 2 | 81-121 |
| Insulin (μU/mL) | 24.1 ± 1.7 | 13.1-46.5 |
| Glucose infusion rate during | 36.0 ± 3.0 | 15.2-60.8 |
| insulin infusion (μmol/kg | | |
| FFM/min) | | |
| Glucose Rd during insulin | 131 ± 19 | 30-355 |
| infusion (% increase) | | |

DATA TABLE 2
Regression coefficient estimates showing the association
between hepatic mRNA expression of genes involved in GABA
production (ABAT) and GABA transport (SLC6A6, SLC6A8, SLC6A12,
and SLC6A13) and basal plasma insulin concentration (μU/mL)
or hepatic insulin sensitivity index (HISI).

| | | | | |
|---|---|---|---|---|
| Estimate | SEM | Lower CI | Upper CI | P- Value |

Basal Plasma Insulin Concentration (μU/mL)

| | | | | | |
|---|---|---|---|---|---|
| Intercept | −52.50 | 40.31 | −143.67 | 38.68 | 0.2251 |
| IHTG (%) | 0.30 | 0.14 | −0.01 | 0.62 | 0.0577 |
| ABAT | 18.14 | 5.28 | 6.19 | 30.09 | 0.0075 |
| SLC6A12 | −14.71 | 4.29 | −24.41 | −5.01 | 0.0075 |
| SLC6A13 | 1.90 | 3.14 | −5.19 | 8.99 | 0.5595 |
| SLC6A6 | 3.02 | 2.40 | −2.41 | 8.45 | 0.2395 |
| SLC6A8 | −1.87 | 1.47 | −5.18 | 1.45 | 0.2342 |

HISI

| | | | | | |
|---|---|---|---|---|---|
| Intercept | 16.41 | 7.23 | 0.06 | 32.76 | 0.0493 |
| IHTG (%) | −0.05 | 0.03 | −0.11 | 0.00 | 0.0674 |
| ABAT | −2.94 | 0.95 | −5.08 | −0.80 | 0.0127 |
| SLC6A12 | 2.38 | 0.77 | 0.64 | 4.12 | 0.0127 |
| SLC6A13 | −0.71 | 0.56 | −1.98 | 0.56 | 0.2379 |
| SLC6A6 | −0.21 | 0.43 | −1.19 | 0.76 | 0.6340 |
| SLC6A8 | 0.17 | 0.26 | −0.43 | 0.76 | 0.5416 |

DATA TABLE 3
Single Nucleotide Polymorphisms (SNPs) in the promoter
of the ABAT gene, which encodes for GABA transaminase, are
associated with a decreased odds ratio (OR) for type 2
diabetes (T2D; Source: knowledge portal diabetes database).
ABAT - T2D Associated SNPs

| | | Predicted | | | | | |
|---|---|---|---|---|---|---|---|
| Variant ID | dbSNP ID | Impact | Study | P-value | Effect | OR | MAF |
| 16_8743360_C_G | rs72768103 | Promotor- | 70KforT2D GWAS | 0.00792 | ↓ | 0.872 | 0.007-0.036 |
| | | intergenic | | | | | |
| 16_8762951_G_A | rs185391944 | Promotor- | DIAGRAM 1000G | 0.0036 | ↓ | 0.852 | 0.005-0.01 |
| | | intergenic | GWAS | | | | |
| 16_8758576_C_G | rs12933032 | Promotor- | UK Biobank T2D | 0.028 | ↓ | 0.962 | 0.1 |
| | | intergenic | GWAS (DIAMANTE- | | | | |
| | | | Europeans September | | | | |
| | | | 2018) | | | | |

MAF—minor allele frequency.

Hepatic ABAT mRNA expression was positively associated with plasma insulin and negatively with HISI (FIGS. 6A-6B). Analysis of single nucleotide polymorphisms (SNPs) reported on the Accelerating Medicines Partnership Type 2 Diabetes Knowledge Portal database shows that SNPs in the reporter region of ABAT are associated with decreased risk of T2D (FIG. 6C; Data Table 3). Importantly, no promoter region SNPs were found that were significantly associated with an increased odds ratio for T2D. Moreover, given the key role of GABA-T in the central nervous system it is not surprising that SNPs in the ABAT gene were not identified. These data support the notion that GABA-T is a driver of insulin resistance in people.

By using ex vivo liver slice model, it was shown that pharmacologically inhibiting BGT1 increased media GABA content, proposing that although this transporter is bidirectional, flux is more heavily driven towards GABA import. Moreover, missense mutations in SLC6A12 are associated with an increased risk of T2DM. In fact, a premature stop codon is associated with a 16-fold increase in the risk for T2DM (Data Table 3). Herein, it is shown that hepatic SLC6A12 mRNA is negatively associated with plasma insulin concentrations and positively associated with HISI (FIGS. 6A and 6B). Without wishing to limit the present invention to any particular theory or mechanism, it is believed that SLC6A12 expression is primarily indicative of GABA re-uptake into hepatocytes, which depresses GABA signaling onto the HVAN.

Since it was established that GABA-T knockdown and inhibition reduces food intake and body weight, the knowledge from the portal diabetes database was used to identify SNPs associated with BMI. Missense mutations in SLC6A12, SLC6A6, and SLC6A13 were found that were associated with increased BMI (Data Table 4). Taken together, the data obtained from the studies conducted in people support the potential clinical translation of these findings in the mouse.

DATA TABLE 4
Sinale Nucleotide Polymorphisms (SNPs) that result in missense mutations in GABA transporters
are associated with BMI (Source: knowledge portal diabetes database).

| | | | | | | | |
|---|---|---|---|---|---|---|---|
| | | Predicted | | | | Effect | |
| Variant ID | dbSNP ID | Impact | Study | P-value | Effect | Size | MAF |

SLC6A12 - BMI Associated SNPs

| | | | | | | | |
|---|---|---|---|---|---|---|---|
| 12_313824_G_A | rs199521597 | Missense: | GIANT 2018 BMI, | 0.0026 | ↑ | 3 | Not |
| | | Replaces | Height exome chip | | | | Reported |
| | | Alanine with | analysis: African | | | | |
| | | Valine | Americans | | | | |
| 12_319125_A_G | rs557881 | Missense: | GIANT UK Biobank | 0.0031 | ↑ | 0.0053 | 0.426 |
| | | Replaces | GWAS | | | | |
| | | Cysteine with | | | | | |
| | | Arginine | | | | | |
| 12_309921_T_C | rs143648821 | Missense: | FUSION exome chip | 0.00316 | ↑ | 0.815 | 0.00105 |
| | | Replaces | analysis | | | | |
| | | Isoleucine with | | | | | |
| | | Valine | | | | | |
| 12_309864_C_T | rs11061915 | Missense: | 13K exome | 0.00623 | ↑ | 0.299 | 0.00354 |
| | | Replaces | sequence analysis | | | | |
| | | Valine with | | | | | |
| | | Isoleucine | | | | | |
| 12_300248_C_G, T | rs147574089 | Missense: | GIANT 2018 BMI, | 0.0081 | ↑ | 0.65 | NaN |
| | | Replaces | Height exome chip | | | | |
| | | Glutamate with | analysis: Hispanics | | | | |
| | | Glutamine | | | | | |
| 12_300298_C_T | rs537332809 | Missense: | 13K exome | 0.0141 | ↑ | 1.09 | 0.000381 |
| | | Replaces | sequence analysis | | | | |
| | | Arginine with | | | | | |
| | | Glutamine | | | | | |

SLC6A6 - BMI Associated SNPs

| | | | | | | | |
|---|---|---|---|---|---|---|---|
| 3_14523209_G_A | rs141254266 | Missense: | 13K exome | 0.00922 | ↑ | 0.866 | 0.00009- |
| | | Replaces | sequence analysis | | | | 0.0003 |
| | | Valine with | | | | | |
| | | Isoleucine | | | | | |
| 3_14523296_G_A | rs41284017 | Missense: | GIANT 2018 BMI, | 0.016 | ↑ | 0.028 | 0.004- |
| | | Replaces | Height exome chip | | | | 0.0165 |
| | | Valine with | analysis | | | | |
| | | Isoleucine | | | | | |
| 3_14526454_G_A | rs200063855 | Missense: | GIANT 2018 BMI, | 0.033 | ↓ | −0.71 | 0.00007- |
| | | Replaces | Height exome chip | | | | 0.0002 |
| | | Arginine with | analysis: South | | | | |
| | | Histadine | Asians | | | | |

SLC6A13 - BMI Associated SNPs

| | | | | | | | |
|---|---|---|---|---|---|---|---|
| 12_332337_C_T | rs202217743 | Missense: | FinnMetSeq exome | 0.0123 | ↑ | 1.26 | 0.0001 |
| | | Replaces | sequence analysis | | | | |
| | | Valine with | | | | | |
| | | Leucine, or | | | | | |
| | | Methionine | | | | | |
| 12_331781_C_T, G | rs147388541 | Missense: | GIANT 2018 BMI, | 0.015 | ↑ | 0.59 | 0.003- |
| | | Replaces | Height exome chip | | | | 0.02 |
| | | Aspartate with | analysis: South | | | | |
| | | Histadine, or | Asians | | | | |
| | | Asparginine | | | | | |
| 12_347102_C_T | rs138506621 | Missense: | GIANT 2018 BMI, | 0.034 | ↑ | 2.1 | 0.00014- |
| | | Replaces | Height exome chip | | | | 0.001 |
| | | Glutamate with | analysis: East Asians | | | | |
| | | Lysine | | | | | |
| 12_352884_C_T | rs543043546 | Missense: | 13K exome | 0.0341 | ↑ | 0.489 | 0.00001- |
| | | Replaces | sequence analysis | | | | 0.00055 |
| | | Glycine with | | | | | |
| | | Serine | | | | | |

MAF—minor allele frequency.

The liver signals in an endocrine fashion through a myriad of hepatokines. Many hepatokines change with the degree of hepatic steatosis and are linked to altered metabolic homeostasis. Liver-produced GABA is added to the list of steatosis affected hepatokines that alter glucose homeostasis. Unlike other hepatokines, which act in an endocrine fashion, GABA is acting locally in a paracrine fashion on the parasympathetic nervous system to mediate its downstream metabolic effects. Muting hepatic GABA production by pharmacologic inhibition or ASO mediated knockdown of GABA-T attenuates obesity-induced hyperinsulinemia and insulin resistance. Beyond the improvements in glucose homeostasis, hepatic GABA-T knockdown decreases food intake causing a decrease in adiposity and weight loss.

Hepatic GABA production improves insulin sensitivity primarily by increasing skeletal muscle glucose clearance (FIGS. 1K and 3G). The results reported herein propose that some of the improvements in glucose clearance may be mediated by increased blood flow. Vasodilation of the microvasculature accounts for 40% of insulin stimulated muscle glucose uptake. Insulin and acetylcholine signaling at endothelial cells stimulates NO production, which signals to smooth muscle cells inducing cGMP production and vasodilation. The microvascular vasodilatory response is reduced in obesity, directly contributing to systemic insulin resistance. EOS treatment increased soleus muscle cGMP concentrations (FIG. 1L).

Although weight loss itself improves insulin sensitivity and metabolic health, GABA-T knockdown improves glucose homeostasis independent of the decrease in food intake and body weight. One week of GABA-T ASO treatment does not decrease food intake (FIG. 4E) or body weight (FIGS. 2C and 4F) compared to controls, yet basal serum insulin concentrations, glucose tolerance, and insulin sensitivity are markedly improved (FIGS. 2D, 2G, and 2J).

The HVAN has long been implicated in transmitting liver derived signals to the hindbrain to regulate feeding behavior. Early work established that hepatic portal infusions of glucose, amino acids, and lipids suppress food intake, while more recent studies support that carbohydrate signals originating from the liver regulate feeding behavior through HVAN dependent mechanisms. Peripheral satiation factors including glucagon like peptide-1 (GLP-1), cholecystokinin (CCK), lipids, and leptin all reduce food intake dependent upon increasing gastric and hepatic vagal branch afferent firing, while the orexigenic hormone ghrelin suppresses vagal afferent tone. A novel addition is proposed to this regulation of vagal nerve activity by metabolites and gut hormones, suggesting that hepatic lipid accumulation stimulates hepatic GABA release to depress HVAN activity. Consistent with the effects of other appetite regulators, GABA mediated suppression of HVAN activity would be expected to increase phagic drive, while removal of this inhibitory signal would be expected to increase HVAN firing and reduce food intake. In lean mice, which have low levels of hepatic GABA release, GABA-T knockdown did not alter food intake. Thus, hepatocyte released GABA represents a novel orexigenic signal enhanced by obesity.

Diet-induced obesity dysregulates the diurnal pattern of food intake. Mice on a chow diet eat ˜20% of their daily food intake during the light cycle while this increases to ˜30% with HFD feeding. In healthy mice, vagal afferent receptor populations are regulated by nutritional state, expressing an orexigenic profile during fasting and an anorexigenic profile upon refeeding. In turn, in chow fed mice, anorexigenic mechano-stimuli more effectively stimulate gastric vagal afferent nerve activity during the light cycle. These circadian patterns are lost with diet-induced obesity. Together these changes promote increased food intake during the light cycle and support the hypothesis that the dysregulation of feeding behavior in obesity is partially mediated by aberrant vagal signaling.

In lean mice, hepatic vagotomy shifts the diurnal feeding pattern to increase light cycle food intake, potentially mediated by the loss of peripheral light cycle anorexigenic stimuli. Diet-induced obese, hepatic vagotomized mice eat less during the light cycle than sham operated controls, suggesting that hepatic vagotomy protects against the obesity-induced increase in daytime feeding. Without wishing to limit the present invention to any particular theory or mechanism, it is believed that preventing the GABA mediated depression of HVAN activity from reaching the hindbrain not only improves glycemic control but decreases light cycle food intake, explaining the decreased weight gain with HFD feeding in hepatic vagotomized mice. Further supporting a role of hepatic GABA in HFD induced weight gain, it was previously observed that inducing hepatic Kir2.1 expression limited hepatic GABA release and HFD induced weight gain.

Weight loss by people with obesity is often difficult to achieve and maintain. It is likely that dysregulated satiety signaling impairs the effectiveness of weight loss strategies. This work identifies hepatic GABA production as a potential therapeutic target which independently improves systemic glucose homeostasis and decreases food intake in obesity. In people with obesity, hepatic GABA production and transport are associated with glucoregulatory markers, T2D, and BMI supporting the potential translation of this work to improve metabolic health in people.

### Example 2 Hepatocyte Membrane Potential Regulates Serum Insulin and Insulin Sensitivity by Altering Hepatic GABA Release

All animal studies excluding those done in albumin-cre expressing mice described below were conducted using male wildtype C57BL/6J purchased from Jackson Laboratories or bred in-house (Bar Harbor, Me.). Albumin-cre male mice were purchased from Jackson Laboratories and crossed with wildtype females to generate in-house breeding of experimental albumin-cre expressing mice (Alb^{Cre/+}) and sibling wildtype mice (Alb^{+/+}). Mice were kept on a 14-hour light/10-hour dark schedule and housed 3-5 mice per cage until 1 week prior to study initiation, at which point animals were individually housed. The studies were conducted in lean chow fed mice (7013 NIH-31, Teklad WI, 3.1 kcal/g, 18% kcal from fat, 59% kcal from carbohydrate, 23% kcal from protein) at 12-16 weeks of age. Studies in diet-induced obese sham and vagotomy mice were performed after 9 weeks on a high fat diet (TD 06414, Teklad WI, 5.1 kcal/g, 60.3% kcal from fat, 21.3% kcal from carbohydrate, 18.4% kcal from protein; 20-26 weeks of age). Studies in obese Kir2.1 expressing mice were performed at 3, 6, and 9 weeks after introduction of the high fat diet and all studies were repeated in 3 different cohorts. Kir2.1 and eGFP expressing mice weighing under 36 grams after 9 weeks of high fat diet feeding were excluded from all data. Unless fasted, mice had ad libitum access to food and water. All studies were approved by the University of Arizona Institutional Animal Care and Use Committee.

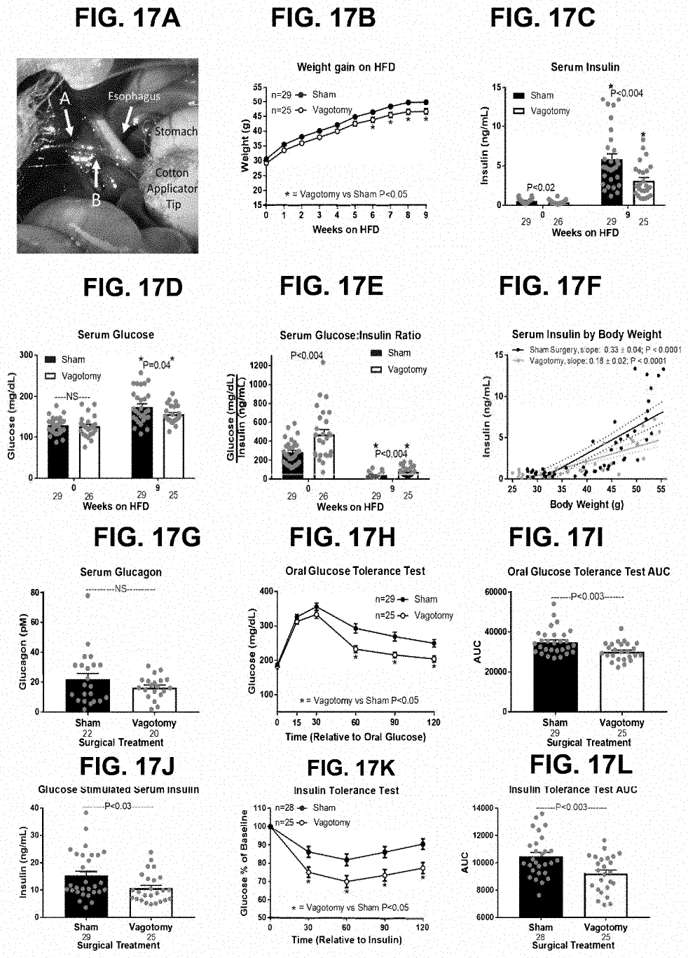

Surgeries were performed in 12-week old male C57BL/6J mice under isoflurane anesthesia. Mice were randomly assigned to a surgical group (sham or vagotomy). A ventral midline incision through the skin and peritoneum allowed us to isolate the hepatic vagus nerve as it branched from the esophagus (FIG. 17A). In vagotomized mice, the hepatic vagal nerve (FIG. 17A; arrow A) was severed while it remained intact in sham operated mice. The peritoneum was sutured with absorbable polyglactin 910 suture and the skin with nylon suture. Mice were given a single post-operative dose of slow release formulated buprenorphine analgesic (1.2 mg/kg slow release, sub-cutaneous). Food intake and body weight were monitored daily and sutures were removed 7 days post-operation.

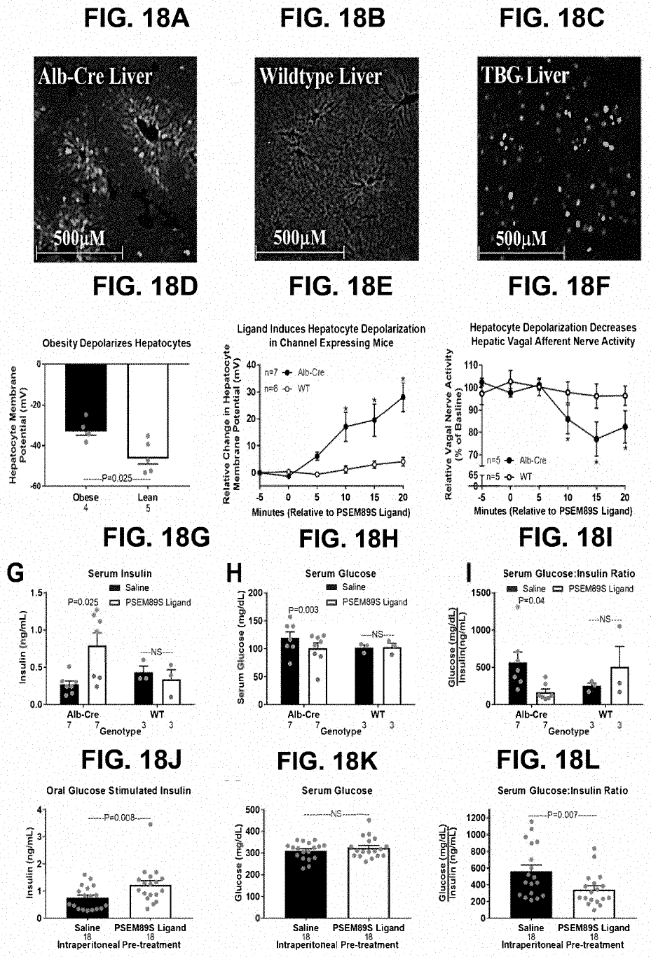

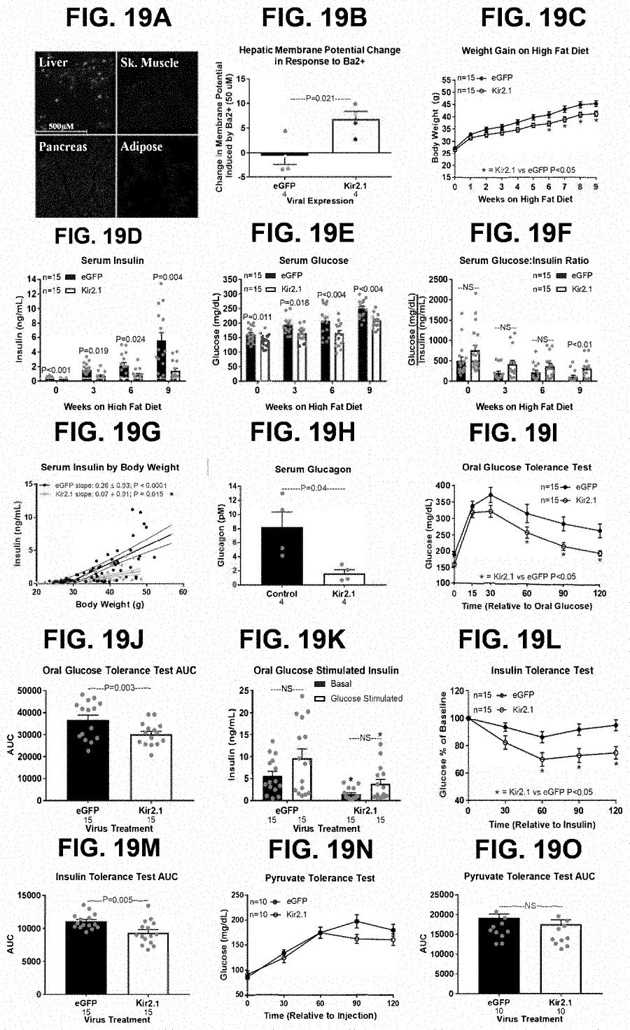

The depolarizing channel (PSAM^{L141F,Y115F}-5 HT3HC), originally engineered by Dr. Scott Sternsons group, was made by mutating the acetylcholine binding domain of a chimeric channel that included the binding domain of the α7 nicotinic acetylcholine receptor and the ion pore domain of the serotonin receptor 3a. The ligand binding domain mutations (Leu¹⁴¹→Phe and Tyr¹¹⁵→Phe) limited the agonist action of acetylcholine and allowed for stimulation by a pharmacologically selective effector molecule PSEM89S. The exogenous ligand PSEM89S opens the serotonin receptor 3a channel allowing Na⁺, K⁺, and Ca⁺⁺ passage into the cell and membrane depolarization. AAV8 viral vectors were used for plasmid delivery in all the reported studies and were synthesized by the Penn Vector Core. Hepatic specific expression of the depolarizing channel was achieved through two different methods. First, expression of a cre-recombinase dependent depolarizing channel was driven by a globally expressed CAG promoter. LoxP sites limited expression to cre-recombinase expressing tissue, and tail vein injection of 1×10¹⁰ viral genome copies established hepatocyte expression in albumin-cre but not wildtype mice. Second, a separate AAV8 viral vector, induced hepatic specific expression of the same depolarizing channel by driving expression using the thyroxine binding globulin (TBG) promoter. Tail vein injection of 1×10¹¹ viral genome copies established hepatocyte expression (FIG. 18C). The thyroxine binding globulin promoter also drove hepatic expression of the hyperpolarizing, inward-rectifier K⁺ channel, Kir2.1. Tail vein injection of 1×10¹¹ viral genome copies established hepatocyte specific expression (FIG. 19A). To confirm channel expression and tissue specificity, all viral vector plasmids encoded for enhanced green fluorescent protein (eGFP).

All studies in virus injected mice were conducted at least 5 days post virus injection to allow for maximal channel expression. Individually housed mice were intraperitoneally injected with the ligand for the depolarizing channel (PSEM89S; 30 mg/kg; 0.1 mL/10 g body weight) or PBS (0.1 mL/10 g body weight).

Studies conducted in mice injected with the cre-dependent depolarizing channel virus took place at 8 am. Food was removed upon study initiation. Blood for serum insulin and glucose determination was collected from the tail vein 15 minutes following intraperitoneal injection. All mice received both saline and PSEM89S ligand injection on separate days.

Studies conducted in mice expressing the cre-independent depolarizing channel began at 1 pm following a 4 hour fast. Mice received an oral glucose gavage (2.5 g/kg) 10 minutes after intraperitoneal injection of the PSEM89S ligand or saline. 15 minutes following glucose administration (25 minutes post treatment injection), blood for serum insulin and glucose determination was collected from the tail vein. All mice received both saline and PSEM89S ligand injection on separate days. These studies were repeated in 2 cohorts.

Simultaneous in vivo recordings were performed of hepatocyte membrane potential and hepatic afferent vagal nerve firing activity in anesthetized (isoflurane) mice to directly assess the effect of hepatocyte depolarization on hepatic afferent vagal nerve activity. The abdomen was shaved and scrubbed with betadine and isopropanol before an incision through the skin and peritoneum was made to expose the internal organs. The intestines were moved to expose the liver and one lobe of the liver was secured onto a small platform to minimize movement caused by respiration. A ground electrode was secured under the skin and the hepatic vagal nerve was gently lifted onto a hook-shaped electrode (FIG. 17A; arrow A) attached to the positive pole of a Grass P511 AC coupled amplifier, and the signal was filtered with a bandwidth of 300-1000 Hz. The nerve and hook electrode were dried and surrounded with ice cold kwik-sil to secure placement of the hook. The hepatic vagal nerve to the right of the hook, near the esophagus was cut to eliminate efferent firing (FIG. 17A; arrow B). Once the kwik-sil had set, the anesthetized mouse was bathed in 37° C. Krebs-Henseleit (KH) buffer gassed with CO2. After placement and sealing of the hook electrode, 45-60 minutes of basal nerve activity was monitored/recorded with pClamp software (version 10.2; Molecular Devices) until nerve activity stabilized, after which, treatments began.

Simultaneously, intracellular recordings of hepatocytes were made with sharp glass electrodes (30-40 MO) pulled from thin-walled borosilicate glass capillary tubes (OD: 1 mm; ID: 0.78 mm; Sutter Instrument Co., Novato, Calif.), filled with 1.5M KCl and positioned visually using a motorized 4-axis micromanipulator (Siskiyou, Grants Pass, Oreg.). Electrical signals were conducted via an Ag—AgCl electrode connected to a headstage (Axoclamp ME-1 probe), which was in turn connected to an Axoclamp 2B amplifier. Both nerve and intracellular signals were sent to an A/D converter (Digidata 1322A, Molecular Devices, Sunnyvale, Calif.), digitized at 20 kHz and viewed on a computer monitor using pClamp software (version 10.2; Molecular Devices).

Before treatments were applied hepatocyte impalement was determined by an abrupt negative deflection upon penetration of the cell and a stable intracellular potential (−45 to −25 mV for mouse hepatocytes) for at least 2 minutes. If the recording of hepatocyte membrane potential was not stable the electrode was removed and membrane potential was measured on another hepatocyte.

To assess the response to channel activation, PSEM89S ligand was bath applied (30 μM) for 45 min during recordings. In order to understand the effect of Kir2.1 channel on hepatocyte membrane potential, a 10-minute baseline measure was collected and then Barium (BaCl 50 μM) was bath applied and recording continued for 45 minutes. Barium blocks Kir2.1 mediated current, thus the change in membrane potential in response to barium indicates the degree of hyperpolarization resulting from Kir2.1 channel expression. In all electrophysiology studies mice were sacrificed by cutting the diaphragm and subsequent cervical dislocation. Tissues were collected to confirm tissue specificity of channel expression and the % of hepatocytes that were expressing the channel. All studies were performed at room temperature (25° C.).

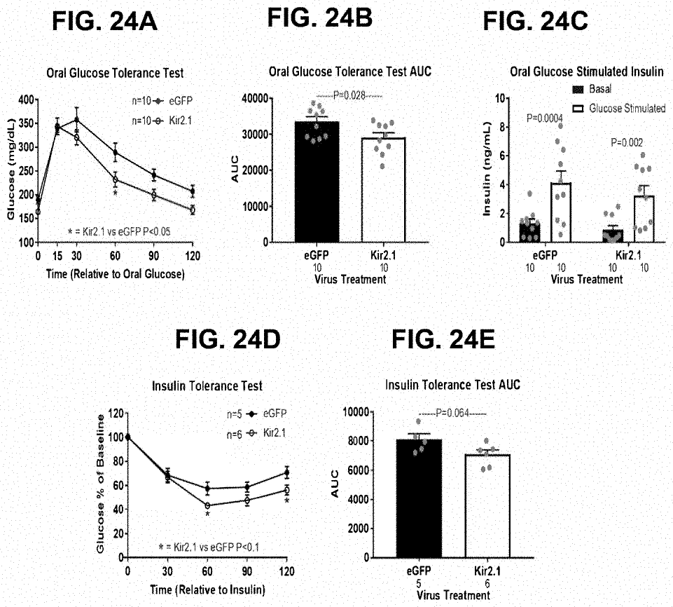

To confirm the specificity and extent of viral-induced channel expression in hepatocytes, immunohistochemistry for GFP was performed. Liver, adipose, pancreas, and skeletal muscle were collected into 4% paraformaldehyde in 0.1 M PBS (Phosphate Buffered Saline) immediately after sacrifice. After 4 h at 4° C., tissues were transferred to a 30% sucrose solution in 0.1 M PBS and kept at 4° C. until tissues sunk to the bottom of the solution. Tissues were snap frozen on liquid nitrogen in OTC (Optimal Cutting Temperature; Sakura Finetek USA Inc, Torrance, Calif.) and stored at −80° C. A cryostat HM 520 (MICROM International GmbH, Walldorf, Germany) was used to get 10 μM thick slices which were collected onto Superfrost Plus slides. Immunohistochemistry for GFP alone (FIGS. 18A-18C, 24A-24B) was performed as follows: Briefly, slides were washed twice in PBS and twice in PBST (3% Triton in PBS) before being exposed to blocking solution (5% normal goat serum in PBST) for 1 h. Slides were subsequently exposed to a 1:5000 dilution of the primary anti-GFP antibody in blocking solution (Alexa488-conjugated rabbit anti-GFP; Life Technologies, Waltham, Mass.) for 3 hours at room temperature. After primary antibody incubation, the slides were washed 3 times in PBST and 2 times in PBS prior to placing the coverslip with DAPI Fluoromount-G as the mounting medium (SouthernBiotech, Birmingham, Ala.). Fluorescent imaging was performed without antibody amplification in mice administered the AAV8 that encoded for Kir2.1 and tdTomato. Immunohistochemistry for GFP and the hepatocyte specific marker arginase-1 was performed as follows: Slides were washed twice in PBS and twice in PBST (3% Triton in PBS) before being exposed to blocking solution (5% normal donkey serum in PBST) for 1 h. Slides were then incubated overnight at 4° C. in a 1:400 dilution of the primary anti-arginase-1 antibody in blocking solution (Rabbit anti-liver arginase ab91279; Abcam, Cambridge, UK). After overnight primary antibody incubation slides were washed 5 times with PBST and exposed to a 1:500 dilution of the primary anti-GFP antibody (Alexa488 conjugated mouse anti-GFP sc-9996 AF488; Santa Cruz, Dallas, Tex.) and the secondary anti-rabbit antibody (Alexa568 conjugated donkey anti-rabbit A10042; Thermo Fisher, Waltham, Mass.) in blocking solution and for 1 hour at room temperature. Slides were then washed 5 times in PBST and 2 times in PBS prior to applying DAPI and a coverslip. Immunohistochemistry for calcitonin gene-related peptide (CGRP) and GABA_{A}, and calretinin and GABA_{A} were performed identical to that described for GFP and arginase-1 with the following primary anti-CGRP and anti-GABA_{A} antibodies at a 1:100 dilution (Goat anti-CGRP ab36001 and rabbit anti-GABA_{A} a5 ab10098; Abcam, Cambridge, UK) and the primary anti-calretinin antibody at a final concentration of 15 μg/mL (Goat anti-calretinin AF5065; R&D Systems, Inc., Minneapolis, Minn.). The secondary anti-rabbit and anti-goat antibodies were used at a 1:500 dilution (Alexa568 conjugated donkey anti-rabbit A10042 and alexa488 conjugated donkey anti-goat A32814; Thermo Fisher, Waltham, Mass.). Images were collected by fluorescent microscopy (Leica DM5500B, Leica Microsystems, Wetzlar, Germany), captured using HCImage Live, and formatted in Image-Pro Premier 9.2. 10× magnification was used to ensure a wide field of vision and accurate assessment of degree of expression. 20× magnification was used to image co-staining for GFP and arginase-1.

Oral glucose (2.5 g/kg; 0.1 mL/10 g body weight; Chem-Impex Intl Inc., Wood Dale, Ill.) was given to 4 hour fasted individually housed mice. All glucose tolerance tests began at 1 pm and glucose was measured in whole blood, collected from the tail vein, by glucometer (Manufacture #D2ASCCONKIT, Bayer, Leverkusen, Germany) at 0, 15, 30, 60, 90, and 120 minutes after glucose gavage. Blood for serum insulin (oral glucose stimulated insulin secretion; OGSIS) and glucose determination was collected from the tail vein 15 minutes following glucose administration.

Intraperitoneal insulin (0.75 U/kg; 0.1 mL/10 g body weight; Sigma Aldrich, St. Louis, Mo.) was given to 4 hour fasted individually housed mice. All insulin tolerance tests began at 1 pm and glucose was measured in whole blood, collected from the tail vein, by glucometer (Manufacture #D2ASCCONKIT, Bayer, Leverkusen, Germany) at 0, 30, 60, 90, and 120 minutes after insulin injection.

Intraperitoneal sodium pyruvate (1.5 g/kg; 0.1 mL/10 g body weight; Alfa Aesar, Ward Hill, Mass.) was given to 16 hour fasted individually housed mice. Mice were switched to wood chip bedding (Harlan Laboratories; Cat. #7090 Sani-Chips) at the initiation of the fast. All pyruvate tolerance tests began at 9 am and the rise in glucose was measured in whole blood, collected from the tail vein, by glucometer (Manufacture #D2ASCCONKIT, Bayer, Leverkusen, Germany) at 0, 30, 60, 90, and 120 minutes after pyruvate injection. This is indicative of hepatic gluconeogenic potential from pyruvate.

Within 2 hours of collection, blood was left to clot at room temperature for 20 minutes. Thereafter the blood was centrifuged at 3,000×g for 30 minutes at 4° C. and serum was collected. Serum was stored at −80° C. until metabolite and hormone analyses. Serum glucose was analyzed by colorimetric assay (Cat. #G7519, Pointe Scientific Inc., Canton Mich.). Serum insulin was analyzed by enzyme-linked immunosorbent assay (ELISA; Cat. #80-INSMSU-E01,E10, Alpco, Salem, N.H.). Serum glucagon was analyzed by enzyme-linked immunosorbent assay (ELISA; Cat. #10-1281-01, Mercodia, Uppsala, Sweden) from tail vein blood collected at 9 am from fed mice (Vagotomy study) or after a 4 hour fast at 1 PM (Kir2.1 study).

Liver slices from experimental mice were incubated ex vivo to measure release of signaling molecules. A peristaltic pump perfusion system was used to deliver warmed KH buffer to the liver through the portal vein. Briefly, mice were anesthetized with an intraperitoneal injection of ketamine (10 mg/mL) and diazepam (0.5 mg/mL). Once mice were unresponsive, an incision in the lower abdomen through the skin and peritoneal membrane was made vertically through the chest along with transverse incisions on both sides to expose the liver. A 30-gauge needle was inserted into the hepatoportal vein to blanch the liver. The inferior vena cava was cut to relieve pressure in the circulatory system and allow blood to drain. The perfusion continued for several minutes at a rate of 4 mL/minute until the liver was completely blanched. The liver was removed and washed in warm PBS before being sliced into 0.2 mm slices using a Thomas Sadie-Riggs Tissue Slicer. Two liver slices were taken from each mouse. Tissue slices were placed individually into a well on a 12-well plate pre-filled with 1 mL of KH buffer that had been sitting in an incubator set to 37° C. and gassed with 5% CO2. Liver slices were incubated in the initial well for 1 hour to stabilize before being transferred to a fresh well pre-filled with KH buffer. Liver slices treated with the GABA-T inhibitor EOS were incubated in media containing EOS (5.3 mM) during the second hour of incubation. Liver slices treated with the Na⁺/K⁺ ATPase inhibitor ouabain were incubated in media containing ouabain (1 mM) media was collected after 15 minutes of incubation. Liver slices treated with the GABA transporter inhibitors for BGT1 and GAT2 were incubated in media containing betaine (1 mM) or Nipecotic acid (1 mM) or both during the second hour of incubation. Liver slices incubated in reduced and low NaCl media sat in normal KH buffer (NaCl 118 mM) for the first hour and then were transferred to reduced (60 mM) or low NaCl (15 mM) KH buffers for the second hour. For the reduced and low NaCl medias, respectively, 58 and 103 mM of NaCl were replaced with 116 and 206 mM of mannitol to maintain the osmolarity of the buffer. After 1 hour in the second well, tissue and media were collected. Liver slice samples and KH media samples from both wells of each mouse were pooled. Liver slices were snap frozen in liquid nitrogen, while media was frozen and stored at −80° C. for future analysis.

Preliminary media samples were sent to the Mayo Clinic Metabolomics Regional Core for mass spectrophotometry analysis using their neuromodulators panel (Data Table 5). For all liver slice GABA and aspartate release data, the media collected from the ex vivo hepatic slice culture was thawed on ice and centrifuged for 5 minutes at 10,000×g at 4° C. to remove tissue debris. GABA was then measured in the supernatant using a commercially available ELISA (REF #BA E-2500, Labor Diagnostika Nord, Nordhorn, Germany).

Aspartate release was measured using liquid chromatography-mass spectrometry. Samples were prepared for analysis by LC-MS/MS using protein precipitation. Twenty μl of each sample and standard curve increment was transferred to 1.5 ml tubes. One hundred eighty μl acetonitrile (ACN) was added to each tube followed by a 5 second vortex. All samples were incubated at 4° C. for one hour for precipitation. Samples were then centrifuged at 10,000 RPM for 10 minutes and the supernatant transferred to 300 μl HPLC vials for analysis. The aqueous portion of the mobile phase was buffered using 10 mM ammonium bicarbonate with the pH adjusted to 7.4 using 1M formic acid and ammonium hydroxide. Methanol was used as the organic portion of the mobile phase. The column for separation was a Phenomenex Luna Silica with 5 μm particle diameter and 100 Å pore size. Column internal diameter was 4.6 mm and length was 150 mm. A Shimadzu LC10 series HPLC with two dual piston pumps was used for sample injection and solvent delivery. The flow rate was fixed at 300 μl per minute. Aspartate was quantified using an LTQ Velos Pro mass spectrometer. Eluate from the Shimadzu HPLC was ionized using a Thermo ESI source. Source voltage was 6 kV; sheath and auxiliary gas flows were 40 and 20 units respectively. The ion transfer capillary was heated to 300° C. The LTQ Velos Pro was operated in negative SRM mode using two transitions: 132.1->115 for quantification and 132.1->88.1 as a qualifier. Data integration and quantification were performed using the Thermo Xcalibur software packaged with the LTQ Velos Pro.

Prior to analysis, frozen livers were powdered using a liquid nitrogen cooled mortar and pestle to obtain homogenous liver samples. To measure liver DNA content (ng dsDNA/g tissue), 10-20 mg of powdered liver was sonicated in 200 μL DEPC H₂O and dsDNA determined by fluorometric assay (Cat. #P7589, Invitrogen, Waltham, Mass.). Whole liver mRNA was isolated from powered liver samples with TRI Reagent® (Life Technologies, Grand Island, N.Y.) and phenol contamination was eliminated by using water-saturated butanol and ether as previously described. cDNA was synthesized by reverse transcription with Verso cDNA synthesis kit (Thermo Scientific, Inc., Waltham, Mass.), and qPCR performed using SYBR 2× mastermix (Bio-Rad Laboratories, Hercules, Calif.) and the Biorad iQ™5 iCycler (Bio-Rad Laboratories, Hercules, Calif.). Expression of β-actin (ACTβ) and GABA-Transaminase (ABAT) mRNA were measured using primers as described previously (Ramakers et al., 2003). LinReg PCR analysis software was used to determine the efficiency of amplification from raw output data. ACTβ served as the reference gene for calculating fold change in gene expression using the efficiency^{-ΔΔCt} method.

Total liver lipids were extracted from powdered liver samples. Briefly, 10-20 mg of sample was sonicated in 1004 PBS. 1 mL of 100% ethanol was added to each sample and vortexed for 10 minutes. Following 5 minutes of centrifugation at 16,000×g at 4° C., supernatant was transferred to a fresh tube for analysis of liver triglycerides (Cat. #T7531, Pointe Scientific Inc., Canton, Mich.).

Hepatic NADH and NAD were quantified by fluorometric assay (ab176723, Abcam, Cambridge, UK). Hepatic ATP concentrations were assessed as previously described.

A total of 19 men and women with obesity who were scheduled for bariatric surgery at Barnes-Jewish Hospital in St. Louis, Mo. participated in this study, which was conducted at Washington University School of Medicine in St. Louis. Subjects provided written, informed consent before participating in this study, which was approved by the Human Research Protection Office at Washington University School of Medicine in St. Louis, Mo. (ClinicalTrials.gov NCT00981500). Intrahepatic triglyceride content was determined by using magnetic resonance imaging (3.0-T superconducting magnet; Siemens, Iselin, N.J.) in the Center for Clinical Imaging Research. A 7-hour (3.5-h basal period and 3.5-h insulin infusion period) HECP, in conjunction with stable isotopically labelled glucose tracer infusion, was then conducted in the Clinical Translational Research Unit (CTRU), as previously described⁵⁵. This procedure was performed to determine whole-body insulin sensitivity, which was assessed as the glucose infusion rate (expressed as mg·kg FFM⁻¹·min⁻¹) during the last 30 minutes of the HECP; and skeletal muscle insulin sensitivity, which was assessed as the percent increase in the rate of glucose disposal above basal, during the last 30 minutes of the HECP.

Liver tissue was obtained by needle biopsy during the bariatric surgical procedure, before any intra-operative procedures were performed. Liver tissue was rinsed in sterile saline, immediately frozen in liquid nitrogen, then stored at −80° C. until RNA extraction. Total RNA was isolated from frozen hepatic tissue samples by using Trizol reagent (Invitrogen, Carlsbad, Calif.). Library preparation was performed with total RNA and cDNA fragments were sequenced on an Illumina HiSeq-4000. The fragments per kilobase million reads upper quartile (FPKM-UQ) values were calculated and used for further gene expression analyses. All RNA-seq data used in this study have been deposited into the NCBI GEO database under accession number GSE144414.

The data were analyzed in SAS Enterprise Guide 7.1 (SAS Inst., Cary, N.C.), using a mixed model ANOVA for all analyses. ANOVA tests do not have a one-tailed vs. two-tailed option, because the distributions they are based on have only one tail. When comparisons between all means were required, a Tukey's adjustment was used for multiple comparisons. When comparisons of means were limited (e.g. only within a timepoint or treatment), a Bonferonni correction was used for multiple comparisons. For the analysis of ITT, OGTT, and PTT repeated measures ANOVA were performed by including time point in the analysis. When applicable analyses were conducted separately for chow and HFD fed mice. In the Kir2.1 mice, which were monitored for response at 0, 3, 6, and 9 weeks, analyses were performed for each timepoint individually. For analysis of cre-dependent depolarizing channel effects, analysis was performed in each genotype separately (Alb^{cre/+} or Alb^{+/+}) and the main effect was injection (PSEM89S ligand or saline). For analysis of the studies using the thyroxine binding promoter driven ligand gated depolarizing channel, the main effect of injection (PSEM89S ligand or saline) was used. For the vagotomy analyses the main effect was surgery (sham or vagotomy) and weeks on high fat diet when applicable. For the Kir2.1 analyses the main effect was virus (eGFP or Kir2.1). Linear regressions of body weight and serum insulin concentrations were performed on Kir2.1 and eGFP controls, and sham and vagotomized mice using SAS Enterprise Guide 7.1. All insulin tolerance tests are presented as a percentage of baseline glucose and additionally presented as raw glucose values. Human data was analyzed using a multivariate regression including IHTG content, and SLC6A6, SLC6A8, SLC6A12, and SLC6A13 mRNA expression as independent variables with Type 3 test of fixed effects used to determine significance and estimates derived from maximum likelihood estimation. All graphs were generated using GraphPad Prism 8 (GraphPad Software Inc., La Jolla, Calif.).

The datasets generated and/or analyzed during the current study are available in the Mendeley data repository at a link provided in the cover letter. Datasets will be made public upon acceptance of this manuscript.

Chronic hepatic vagotomy eliminates the ability of the liver to alter vagal afferent nerve activity. However, hepatic vagotomy does not prevent otherwise basal signaling of the vagus in the central nervous system. In fact, basal signaling at the nucleus of the solitary tract (NTS) is restored within 1 month of surgery.

Without wishing to limit the present invention to any particular theory or mechanism, it is believed that hepatic lipid accumulation drives hyperinsulinemia and insulin resistance by altering HVAN activity. It was examined whether or not obesity-induced insulin dysregulation is dependent on an intact hepatic vagal nerve. It was expected that hepatic vagotomy would mute obesity-induced hyperinsulinemia and insulin resistance. Hepatic vagotomy or sham surgery was performed in lean mice and then provided them a 60% high fat diet (HFD; Teklad, TD 06414) for 9 weeks. The operative field is visualized in FIG. 17A, with arrow A indicating the hepatic vagal branch which was severed to hepatic vagotomize mice (FIG. 17A). Vagotomy suppressed weight gain on a HFD starting at week 6 (FIG. 17B). Hepatic vagotomy lowered serum insulin and elevated the glucose:insulin ratio at both 0 and at 9 weeks on the HFD, while decreasing serum glucose concentrations after 9 weeks of HFD feeding (FIG. 17C-17E). For the same increase in body weight during HFD feeding, the rise in serum insulin was greater in sham than vagotomized mice (FIG. 17F). Thus, the vagotomy induced protection against obesity-induced hyperinsulinemia is not due to limited weight gain on the HFD. Serum glucagon concentrations in HFD fed mice were not different between surgical treatments (FIG. 17G).

Vagotomy improved oral glucose tolerance at 9 weeks on the HFD, while simultaneously decreasing glucose stimulated insulin concentrations (FIGS. 17H-17J). Vagotomy also improved insulin sensitivity in obese mice (FIGS. 17K-17L). These data support the conclusion that surgically interrupting hepatic vagal signaling attenuates the development of diet-induced hyperinsulinemia and insulin resistance.

Obesity depolarizes hepatocytes (FIG. 18D). It was hypothesized that obesity-induced hepatocyte depolarization is communicated through the HVAN to dysregulate insulin secretion and action. The genetically-engineered, PSEM89S ligand-gated depolarizing ion channel was used as described by Magnus, et al. (2011), to assess the effect of hepatocyte depolarization on HVAN firing activity. An adeno-associated virus serotype 8 (AAV8) encoding this PSEM89S ligand-gated depolarizing channel and green fluorescent protein (eGFP) flanked by LoxP sites to wildtype mice or mice expressing cre-recombinase driven by the albumin promoter was intravenously delivered. This channel will only be expressed in hepatocytes of cre-recombinase expressing mice and activated by PSEM89S ligand. Immunohistochemistry was performed against GFP to confirm liver specific expression in albumin-cre expressing mice and no expression in wildtype mice (FIGS. 18A-18B). No GFP expression was observed in skeletal muscle, pancreas, or adipose tissue of albumin-cre mice. To assess the influence of hepatocyte depolarization on HVAN firing, hepatocyte membrane potential and HVAN activity was simultaneously measured in the anesthetized mouse. To record HVAN firing activity, the hepatic vagal nerve was gently lifted and placed onto a hook-shaped electrode (FIG. 17A; arrow A). After the electrode placement was secured, the hepatic vagal nerve to the right of the hook near the esophagus (FIG. 17A; arrow B) was cut to eliminate efferent firing activity. Bath application of the PSEM89S ligand (30 μM) depolarized hepatocytes and decreased HVAN firing activity in albumin-cre, channel expressing mice (FIGS. 18E-18F), while having no effect in wildtype mice (FIGS. 18E-18F).

Hepatocyte depolarization depresses HVAN activity (FIGS. 18E-18F), while loss of HVAN signaling in obesity protects against the development of hyperinsulinemia (FIG. 17C). Therefore, it is hypothesized that hepatocyte depolarization caused hyperinsulinemia by altering HVAN activity. To directly test this causative relationship, PSEM89S ligand (30 mg/kg) was intraperitoneally injected to induce hepatocyte depolarization and showed that this more than doubled serum insulin and decreased serum glucose concentrations in albumin-cre, channel expressing mice (FIGS. 18G-18H). Accordingly, PSEM89S ligand decreased the glucose:insulin ratio in albumin-cre mice (FIG. 18I). Notably, PSEM89S ligand did not alter serum insulin, glucose, or the glucose:insulin ratio in wildtype mice (FIGS. 18G-18I).

We developed a second model of hepatocyte depolarization in which liver specific expression of the same PSEM89S ligand-gated depolarizing channel and GFP was independent of cre-recombinase and instead driven by the liver specific thyroxine binding globulin (TBG) promoter 17,18. Wildtype mice intravenously injected with this AAV8 had liver specific GFP expression confirmed by immunohistochemistry (FIG. 18C). No GFP expression was observed in skeletal muscle, pancreas, or adipose tissue. To further validate that the GFP positive cells in the liver are hepatocytes, double immunohistochemistry was performed for GFP and the hepatocyte specific marker, arginase-1. To ensure stimulatory concentrations of circulating glucose, an oral glucose gavage was given (2.5 g/kg body weight) 10 minutes following intraperitoneal PSEM89S ligand (30 mg/kg) injection. As previously observed, PSEM89S ligand administration elevated serum insulin and lowered the glucose:insulin ratio in channel expressing mice (FIGS. 18J and 18L). PSEM89S ligand injection did not affect the rise in serum glucose following an oral gavage of glucose (FIG. 18K). These data establish that acute hepatocyte depolarization depresses HVAN firing activity and increases serum insulin concentrations.

Having established that hepatocyte depolarization increases serum insulin concentrations (FIGS. 2A-K), and that the hepatic vagus is essential for diet-induced hyperinsulinemia (FIG. 17C), it is hypothesized that hepatocyte hyperpolarization would prevent obesity-induced hyperinsulinemia. To induce a chronic hyperpolarized state an AAV8 vector, was used encoding TBG promoter driven expression of the K+ channel, Kir2.1, and eGFP (FIG. 18A). Although this channel is inwardly rectifying in neurons, in hepatocytes, with a resting membrane potential that ranges from −20 to −50 mV, Kir2.1 expression supports K+ efflux and hyperpolarization 19. The hyperpolarizing effect of Kir2.1 was confirmed by in vivo intracellular measurement of hepatocyte membrane potential before and after bath application of the Kir2.1 antagonist, Barium (Ba2+; 50 μM) 19. Ba2+ induced a 6.86±1.54 mV depolarization of hepatocytes in Kir2.1 expressing mice but had no effect (−0.62±1.86 mV) in control eGFP expressing mice (FIG. 19B).

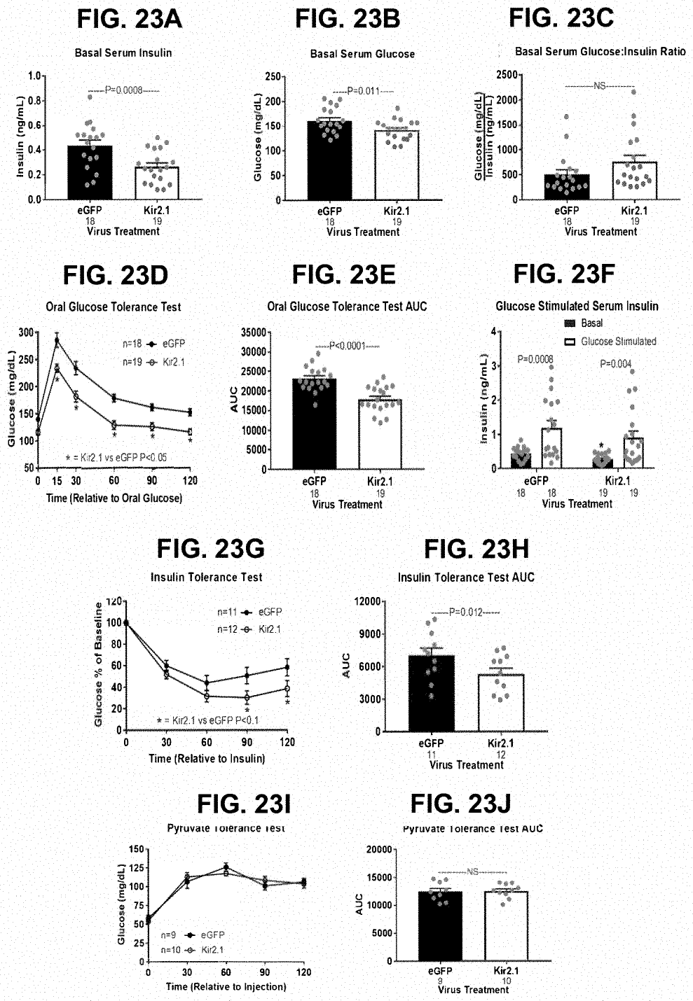

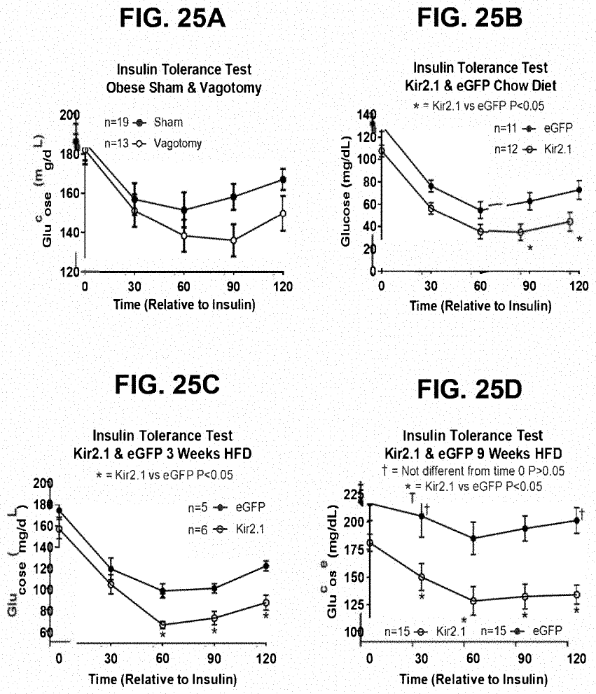

In lean mice, hepatocyte hyperpolarization decreased basal serum insulin and glucose concentrations (FIG. 23A-25C), improved glucose clearance (FIG. 23D-25F) and enhanced insulin sensitivity (FIGS. 23G-H). Kir2.1 expression did not affect gluconeogenic potential, as assessed by a pyruvate tolerance test (FIGS. 23I-J). This establishes that hepatocyte membrane potential affects systemic glucose homeostasis in non-disease, non-obese conditions.

Kir2.1 expression depressed weight gain on a HFD, reaching significance from weeks 6-9 (FIG. 19C). As observed in lean mice, the beneficial response to hepatocyte hyperpolarization was also observed at 3 weeks on a HFD, when body weight remained similar. At 3 weeks on the HFD, Kir2.1 expression improved glucose clearance without altering glucose stimulated serum insulin (FIGS. 24A-C). Kir2.1 expression tended to improve insulin sensitivity at 3 weeks of HFD feeding (P=0.064; FIGS. 24D-E). Kir2.1 expression limited the rise in serum insulin and glucose in response to 3, 6, or 9 weeks of HFD feeding, and increased the glucose:insulin ratio after 9 weeks on the HFD (FIGS. 19D-19F). Although Kir2.1 expression limited HFD-induced weight gain, the same increase in body weight led to a greater increase in serum insulin concentration in eGFP control than in Kir2.1 expressing mice (FIG. 19G). Kir2.1 expression decreased serum glucagon in diet-induced obese mice (FIG. 19H). After 9 weeks on the HFD, Kir2.1 expression improved glucose tolerance and insulin sensitivity (FIGS. 19I-19M), while having no effect on gluconeogenic potential from pyruvate (FIGS. 19N-19O). These results suggest that hepatocyte hyperpolarization protects against the development of hyperinsulinemia, hyperglucagonemia, hyperglycemia, glucose intolerance, and insulin resistance in diet-induced obesity.

Importantly, Kir2.1 expression did not affect HFD-induced hepatic lipid accumulation (Kir2.1: 94.2±10.6 mg triglycerides/g liver versus eGFP control: 98.4±6.6 mg triglycerides/g liver; P=0.73). The absence of hyperinsulinemia in obese Kir2.1 expressing mice despite the development of hepatic steatosis supports hepatocyte depolarization as a critical mediator in the relationship between hepatic lipid accumulation and dysregulated glucose homeostasis.

Resolving the mechanism by which hepatocyte membrane potential can alter HVAN activity and downstream glucose homeostasis is critical to understanding the link between fatty liver and insulin dysregulation. To investigate whether the liver releases neurotransmitters to affect HVAN activity, liver slices were incubated ex vivo and measured the release of neurotransmitters into the media (Data Table 5).

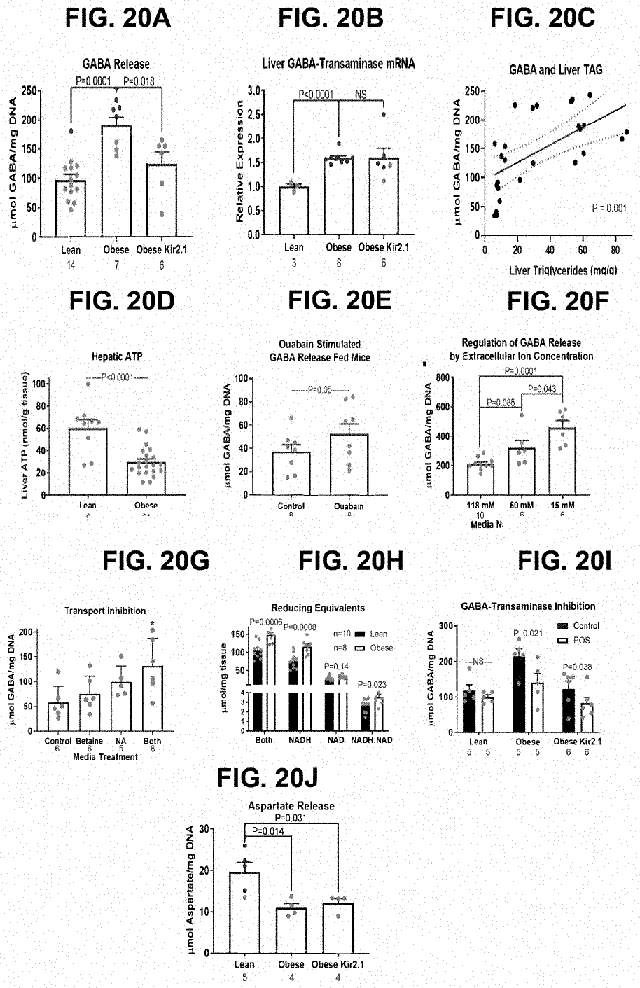

Since obesity depolarizes hepatocytes (FIG. 18D), and hepatocyte depolarization decreases HVAN firing activity (FIGS. 18E-18F), it is hypothesized that obese livers would display either an increase in the release of inhibitory or a decrease in the release of excitatory neurotransmitters. Liver slices from obese mice released more of the inhibitory neurotransmitter GABA than liver slices from lean mice (FIG. 20A). Hepatocytes synthesize GABA via the mitochondrial enzyme GABA-Transaminase (GABA-T) 20. Hepatic GABA-T mRNA expression was increased in diet-induced obesity (FIG. 20B). By measuring liver triglyceride concentration in the same livers from which had measured media GABA concentrations, it is shown that with an increase in liver triglyceride concentration there is increased media GABA concentration (FIG. 20C).

Diet-induced obesity decreased hepatic ATP concentrations (FIG. 20D) and lowers hepatic activity of the Na+/K+ ATPase 21. Without wishing to limit the present invention to any particular theory or mechanism, it is proposed that increased intracellular sodium ions and hepatocyte depolarization resulting from diminished Na+/K+ ATPase activity in obesity promotes GABA efflux (FIG. 21). Diet-induced obesity decreased in hepatic ATP concentrations (nmol/g tissue) to less than 50% of that seen in lean mice (FIG. 20D). Pharmacologically inhibiting Na+/K+ ATPase activity in the liver slice by bath application of Ouabain increased GABA release (μmol/mg DNA) by more than 40% (FIG. 20E). Conversely, Kir2.1 expression, which limited obesity-induced hepatocyte depolarization, decreased obesity-induced hepatocyte slice GABA release without altering the obesity-induced increase in GABA-T mRNA expression (FIGS. 20A-20B). Having established a key role of membrane potential in the regulation of hepatic slice GABA release and re-uptake, it is aimed to better understand hepatic GABA transport.

Given the role of membrane potential in regulating media GABA concentrations, it is proposed that liver slice GABA import and export may be mediated by ion dependent transporters. The liver expresses 4 electrogenic GABA transporters that are members of the Na⁺/Cl--dependent neurotransmitter transporter (SLC6) family. These include proteins encoded for by Slc6A12 (Betaine GABA transporter 1, BGT1), Slc6A13 (GABA transporter 2, GAT2), Slc6A6 (Taurine Transporter, TauT), and Slc6A8 (Creatine transporter, CRT). BGT1 and GAT2 both co-transport 3 Na⁺, 1 Cl- and GABA, moving 2 positive charges in the direction of GABA transport. TauT co-transports 2.5 Na⁺, 1 Cl- and GABA, moving 1.5 positive charges in the direction of GABA transport. The CRT transporter co-transports 2 Na⁺, 1 Cl- and GABA (or creatine) moving a single positive charge in the direction of GABA transport. To establish the role of these ion dependent transporters in GABA export it is shown that hepatic slice GABA release was encouraged by incubation in media with low NaCl concentrations (FIG. 20F). It is also shown that incubation with the BGT1 and GAT2 inhibitors, betaine (1 mM) and nipecotic acid (NA; 1 mM), respectively, increases media GABA concentrations (FIG. 20G), establishing their redundant, but key role in hepatic GABA re-uptake (FIG. 21). As the liver is a primary sight of creatine production, it is evident that the CTR transporter can work against hepatic membrane potential to export its cargo. In fact, the reversal potential for CRT is −26 mV, well within the range of membrane potentials reported in obese mice. The results from these studies establish the key role of membrane potential in regulating activity of these electrogenic GABA transporters results to balance GABA release and re-uptake (FIG. 21).

GABA-T is most frequently thought to be an enzyme key to GABA breakdown. However, early in vitro studies established that the reaction in the direction of GABA synthesis was favored with a Keq 0.04 25. The reason that this reaction most frequently proceeds in the reverse direction is a lack of succinate semialdehyde (SSA), for which SSA dehydrogenase (SSADH) has a nearly 10×lower Km than GABA-T. In the model (FIG. 21), it is proposed that the elevated NADH:NAD ratio observed in obesity favors the production of succinate semialdehyde and ultimately GABA (FIG. 20H). To establish the key role of GABA-T activity in hepatocyte GABA release, liver slices were treated with the irreversible GABA-T inhibitor, ethanolamine-O-sulphate ex vivo (EOS; 5.3 mM). EOS decreased GABA export from obese control and obese Kir2.1 expressing liver slices, but not liver slices from lean mice (FIG. 20I). This supports the hypothesis that GABA production is elevated in obesity and that GABA-T mediated synthesis of GABA is not impaired by Kir2.1 expression. Hepatocytes from obese mice also released less of the excitatory neurotransmitter, aspartate, than hepatocytes from lean mice (FIG. 20J). This model proposes that this decrease in aspartate is a direct effect of the GABA-T stimulated α-ketoglutarate synthesis and transamination to produce oxaloacetate to support gluconeogenic flux (FIG. 21). There was no effect of Kir2.1 expression on the obesity-induced decrease in aspartate release from liver slices (FIG. 20J). As hepatic Kir2.1 expression is able to prevent the hyperinsulinemia and insulin resistance in obesity (FIGS. 19D and 19L) but does not affect the decrease in hepatic slice aspartate release, this proposes that a decreased excitatory signal at the HVAN in obesity is not responsible for the development of insulin resistance.

Vagal afferent innervation in the liver has previously been identified using the vagal sensory immunohistochemical marker calretinin. Calcitonin gene-related peptide (CGRP) has also been proposed as a marker of hepatic vagal afferent innervation and hepatic CGRP staining is eliminated by capsaicin treatment and substantially reduced following bilateral vagotomy. The presence of both calretinin and CGRP positive innervation is confirmed in the mouse liver and established the presence of GABAA receptors on calretinin and CGRP immunoreactive neurons. Consistent with previous reports, the strongest degree of vagal afferent staining was evident in periportal areas while immunoreactive fibers penetrate into the liver parenchyma. Without wishing to limit the present invention to any particular theory or mechanism, it is believed that elevated hepatic GABA release activates GABAA receptors causing chloride influx into vagal afferents explaining the decrease in HVAN activity in response to hepatocyte depolarization.

To understand the potential clinical relevance of these findings, the hepatic mRNA expression of GABA transporters (SLC6A6, encodes for taurine transporter, TauT; SLC6A8, encodes for the creatine transporter, CRT; SLC6A12, encodes for the Betaine-GABA Transporter 1, BGT1; and SLC6A13, encodes for GABA Transporter 2, GAT2) was assessed in 19 people with obesity (age 45±3 yrs old, 2 men and 17 women) who were carefully characterized by measuring intrahepatic triglyceride (IHTG) content using magnetic resonance imaging (MRI) and insulin sensitivity using the hyperinsulinemic-euglycemic clamp procedure (HECP) in conjunction with stable isotopically labeled glucose tracer infusion. The subjects had a wide range in IHTG content, plasma insulin concentration and measures of insulin sensitivity (Data Table 6). The multivariate regression shown that IHTG (%) was negatively associated with both glucose infusion rate during a clamp (FIG. 22A) and the percent increase in glucose rate of disposal from the basal state to the hyperinsulinemic clamp (FIG. 22B and Data Table 7). Similarly, hepatic SLC6A6 (Tau-T) and SLC6A8 (CRT) mRNA expression were negatively related to glucose infusion rate and insulin-induced percent increase in glucose disposal (FIGS. 22A-B). Finally, hepatic SLC6A12 and SLC6A13 expression were positively related to glucose infusion rate and insulin stimulated enhancement of glucose disposal (FIGS. 22A-B). In turn, without wishing to limit the present invention to any particular theory or mechanism, it is believed that BGT1 and GAT2 are primarily acting as GABA re-uptake transporters and that Tau-T and CRT are acting to export GABA. This hypothesized role of BGT1 and GAT2 in hepatic GABA re-uptake is supported by the explant data (FIG. 4G). Of note, the summed expression of SLC6A6 and SLC6A8 was positively associated with IHTG, suggesting that their expression increases with steatosis (P=0.04). Again, with a reversal potential of −26 mV CRT is most likely to be a membrane potential sensitive GABA exporter (FIG. 18D).

The Accelerating Medicines Partnership Type 2 Diabetes Knowledge Portal was used to understand the effect of missense mutations in genes encoding the hepatic GABA transporters (SLC6A6, SLC6A8, SLC6A12, and SLC6A13). Missense mutations in the SLC6A12 and SLC6A13 genes are significantly associated with an increased incidence of T2D (FIG. 22C; Data Table 7). Of note a missense mutation inducing a pre-mature stop codon in the SLC6A12 increases the odds ratio for T2D 15.8 times, establishing its key role in limiting the incidence of diabetes. Although not specific to the liver, this GWAS data does support the hypothesis that GABA transporters encoded by SLC6A12 and SLC6A13 act to preventing the development of T2D.

DATA TABLE 5
Liver slice neurotransmitter panel data

| Neurotransmitter | Lean | Obese | % Change in |
|---|---|---|---|
| (μmol/μg DNA) | (N = 5) | (N = 3) | Obesity |
| Adenosine | 0.22 ± 0.04 | 0.10 ± 0.01 | −55%* |
| Histidine | 17.74 ± 0.92 | 12.90 ± 0.72 | −27%* |
| Serine | 22.32 ± 3.33 | 13.02 ± 0.53 | −42% |
| Taurine | 238.40 ± 18.41 | 305.18 ± 38.04 | 28% |
| Glutamine | 49.06 ± 5.19 | 40.39 ± 3.98 | −17% |
| Glycine | 130.74 ± 5.16 | 81.31 ± 4.93 | −37%* |
| Aspartic Acid | 6.92 ± 0.55 | 3.47 ± 0.32 | −50%* |
| Glutamic Acid | 30.32 ± 2.12 | 28.74 ± 3.48 | −5.2% |
| GABA | 5.43 ± 0.64 | 8.77 ± 0.53 | 61%* |

Initial neuromodulators panel analysis on media collected form the liver explant studies performed by the Mayo Clinic Metabolomics Regional Core.
*Indicates significant difference between obese and lean mice (P < 0.05). Data are presented as mean ± SEM.

DATA TABLE 6
Metabolic characteristics of the study subjects (n = 19).

| | Mean ± SEM | Range |
|---|---|---|
| Body mass index (kg/m²) | 45.1 ± 1.3 | 35.9-55.6 |
| Intrahepatic | 11.4 ± 1.9 | 2.7-28.0 |
| triglyceride content (%) | | |
| Glucose (mg/dL) | 97 ± 2 | 81-121 |
| Insulin (μU/mL) | 24.1 ± 1.7 | 13.1-46.5 |
| Glucose infusion rate during | 36.0 ± 3.0 | 15.2-60.8 |
| insulin infusion | | |
| (μmol/kg FFM/min) | | |
| Glucose Rd during | 131 ± 19 | 30-355 |
| insulin infusion (% increase) | | |

FFM, fat free mass; Glucose Rd, glucose disposal rate.

DATA TABLE 7
Regression coefficient estimates showing the association
between hepatic mRNA expression of genes involved in
GABA production (ABAT) and GABA transport (Slc6A6, A8,
A12, and A13) and glucose infusion rate (μMol/Kg Fat
Free Mass/min) and Glucose Rd (rate of disposal; % increase)
during a hyperinsulinemic-euglycemic clamp.

| | | | | |
|---|---|---|---|---|
| Estimate | SEM | Lower CI | Upper CI | P- Value |

Glucose Infusion Rate (μMol/Kg Fat Free Mass/min)

| | | | | | |
|---|---|---|---|---|---|
| Intercept | −36.41 | 53.75 | −158.00 | 85.18 | 0.5152 |
| IHTG (%) | −0.50 | 0.19 | −0.92 | −0.08 | 0.0242 |
| SLC6A12 | 13.80 | 5.72 | 0.86 | 26.74 | 0.0391 |
| SLC6A13 | 10.74 | 4.18 | 1.28 | 20.20 | 0.0302 |
| SLC6A6 | −15.63 | 3.20 | −22.87 | −8.38 | 0.0009 |
| SLC6A8 | −5.65 | 1.95 | −10.07 | −1.23 | 0.0179 |
| ABAT | −3.26 | 7.04 | −19.20 | 12.67 | 0.6545 |

Glucose Rd During Insulin Infusion (% Increase)

| | | | | | |
|---|---|---|---|---|---|
| Intercept | −3.91 | 4.24 | −13.49 | 5.68 | 0.3805 |
| IHTG (%) | −0.03 | 0.01 | −0.07 | 0.00 | 0.0427 |
| SLC6A12 | 1.02 | 0.45 | 0.00 | 2.04 | 0.0505 |
| SLC6A13 | 0.64 | 0.33 | −0.10 | 1.39 | 0.0834 |
| SLC6A6 | −0.71 | 0.25 | −1.28 | −0.14 | 0.0204 |
| SLC6A8 | −0.45 | 0.15 | −0.79 | −0.10 | 0.0178 |
| ABAT | −0.24 | 0.56 | −1.50 | 1.01 | 0.6712 |

DATA TABLE 8
Single nucleotide polymorphisms (SNPs) that result in missense mutations in GABA transporters are associated with
an increased incidence (OR; odds ratio) of type 2 diabetes (T2D; source: knowledge portal diabetes database).

| | | | | | | | |
|---|---|---|---|---|---|---|---|
| | | Predicted | | | | | |
| Variant ID | dbSNP ID | Impact | Study | P-value | Effect | OR | MAF |

SLC6A12 - T2D Associated SNPs

| | | | | | | | |
|---|---|---|---|---|---|---|---|
| 12_313839_G_A | rs188610 | Missense: | AMP T2D-GENES T2D | 0.0238 | ↑ | 1.1 | 0.0386 |
| | | synonymous | exome sequence | | | | |
| | | variant | analysis | | | | |
| 12_313824_G_A | rs199521597 | Missense: early | BioMe AMP T2D GWAS | 0.0409 | ↑ | 15.8 | 0.000269 |
| | | stop codon | | | | | |

SLC6A13 - T2D Associated SNPs

| | | | | | | | |
|---|---|---|---|---|---|---|---|
| 12_330193_C_T | rs61741313 | Missense: | DIAMANTE (European) | 0.04 | ↑ | 1.04 | 0.01291- |
| | | Replaces Arganine | T2D GWAS | | | | 0.0531 |
| | | with Glutamine | | | | | |

SLC6A6 -T2D Associated SNPs

| | | | | | | | |
|---|---|---|---|---|---|---|---|
| 3_14489107_G_A | rs62233560 | Missense: | AMP T2D-GENES T2D | 0.00143 | ↑ | 1.4 | 0.005- |
| | | Replaces Valine | exome sequence | | | | 0.0165 |
| | | with Isoleucine | analysis: Europeans | | | | |
| 3_14523296_G_A | rs41284017 | Missense: | 70KforT2D GWAS | 0.00234 | ↑ | 1.38 | 0.0062- |
| | | Replaces Valine | | | | | 0.0165 |
| | | with Isoleucine | | | | | |

SLC6A12 - T2D Adjusted for BMI Associated SNPs

| | | | | | | | |
|---|---|---|---|---|---|---|---|
| 12_302492_C_G | rs138178078 | Missense: | ExTexT2D exome chip | 0.000071 | ↑ | 1.26 | 0.0052 |
| | | Replace | analysis | | | | |
| | | Tryptophan with | | | | | |
| | | Serine | | | | | |
| 12_319125_A_G | rs557881 | Missense: | ExTexT2D exome chip | 0.0396 | ↑ | 1.01 | 0.48 |
| | | Replace Cysteine | analysis | | | | |
| | | with Arginine | | | | | |
| 12_300248_C_G, T | rs147574089 | Missense: | CAMP GWAS | 0.0397 | ↑ | 6.09 | 0.0012 |
| | | Replace Glutamate | | | | | |
| | | with Glutamine | | | | | |

SLC6A13 - T2D Associated SNPs

| | | | | | | | |
|---|---|---|---|---|---|---|---|
| 12_346454_C_T | rs140951084 | Missense in Splice | BioMe AMP T2D GWAS | 0.0359 | ↑ | 3.55 | 0.0019 |
| | | Region: Replace | | | | | |
| | | Arginine with | | | | | |
| | | Glutamine | | | | | |

MAF—minor allele frequency.

Reported herein, novel neurotransmitter signaling by which hepatic steatosis may induce systemic insulin dysregulation, while establishing that hepatocyte GABA release is regulated by hepatocyte membrane potential. Using surgical and viral mouse models, the key role of hepatic GABA production and release and hepatic vagal nerve signaling have been established in the dysregulation of glucose homeostasis in obesity.

The present invention features a new model that links hepatic lipid accumulation to HVAN activity and the development of hyperinsulinemia and insulin resistance (FIG. 5). Hepatic lipid accumulation increases flux through gluconeogenesis and increases the hepatic FADH:FAD and NADH:NAD ratio (FIG. 4H). The altered hepatic redox state inhibits the conversion of succinate to fumarate in the TCA cycle and instead drives succinate to succinate semialdehyde. Succinate semialdehyde serves as substrate for GABA-T mediated GABA production. Together with aspartate, the α-ketoglutarate formed by GABA-T produces oxaloacetate and glutamate, which feeds back into the GABA-T catalyzed reaction. The demand for gluconeogenic substrate and the high NADH:NAD ratio drives the carbons in oxaloacetate to malate and through gluconeogenesis. This gluconeogenic drive increases aspartate metabolism, explaining the decreased aspartate release in liver slices from obese mice (FIG. 4J). Thus, gluconeogenic flux and a more reduced mitochondrial redox state direct the flow of intermediate molecules in obesity resulting in elevated hepatic GABA production and increased aspartate utilization.

The ion dependence of GABA transport makes hepatocyte GABA export sensitive to changes in membrane potential. Since GABA transporters are sodium co-transporters, an increase in intracellular sodium ions and hepatocyte depolarization increases GABA export (FIG. 4E). Obesity decreases hepatic ATP content (FIG. 4D) and lowers Na+/K+ ATPase activity 21, providing a mechanism by which obesity depolarizes hepatocytes 12 (FIG. 2D) and encourages GABA export (FIG. 4A). In fact, type II diabetics have lower hepatic ATP concentrations, and both peripheral and hepatic insulin sensitivity is significantly correlated with liver ATP concentrations.

The model proposes that hepatic lipid accumulation ultimately increases hepatic GABA signaling through two separate mechanisms. First, hepatic GABA production is stimulated as a result of increased GABA-T expression (FIG. 4B) and gluconeogenic flux (FIG. 5), and second, hepatic GABA release is stimulated by hepatocyte depolarization (FIG. 4E). This model provides rationale to explain why gluconeogenesis and hepatocyte depolarization are essential to the development of insulin resistance and hyperinsulinemia.

This research highlights the role of hepatocyte depolarization in diet-induced metabolic dysfunction. Under physiologic conditions, hepatocyte membrane potential is closely regulated by insulin and glucagon. Acutely, insulin depolarizes while glucagon hyperpolarizes hepatocytes 33,34. The hyperpolarizing effect of glucagon is proposed to be mediated by cAMP and is dependent on the Na+/K+ ATPase 33. Accordingly, cAMP or glucagon counteracts insulin stimulated hepatocyte depolarization 35,36 High fat diet feeding decreases hepatic cAMP content in mice 37. The decrease in hepatic cAMP along with diminished ATP may contribute to obesity-induced hepatocyte depolarization. Interestingly, a loss of hepatocyte membrane polarity has been implicated in the pathology of other disease states. Hepatocellular carcinoma is characterized by hepatocyte depolarization and increased GABAergic signaling, while increasing hepatocyte polarization protects against tumor proliferation 38. Thus, the regulation of hepatocyte membrane potential in healthy and disease states is critically tied to cellular and metabolic function.

Physiological concentrations of insulin and glucagon induce a 5-7 mV change in hepatocyte membrane potential. This is comparable to the 6.86±1.54 mV hyperpolarization induced by Kir2.1 expression (FIG. 3B). Admittedly, the PSEM89S ligand maximally depolarized hepatocytes by 28±5.4 mV (FIG. 2E), which exceeds the depolarization observed in obesity (13±4.7 mV; FIG. 2D), and likely represents a supraphysiological response. However, at 10 minutes after PSEM89S ligand administration, when HVAN activity was first significantly depressed, hepatocyte depolarization was 17.0±5.4 mV, representing a more physiological change in membrane potential.

Although the idea that liver derived signals communicate to the central nervous system via the HVAN is well established in the literature, the degree hepatocyte vagal afferent innervation has remained controversial. Provided herein is evidence of vagal sensory innervation in close proximity to hepatocytes and have established the presence of GABAA receptors on both calretinin and CGRP immunoreactive neurons in the liver (FIGS. 6A-D). Once exported, hepatic GABA can act at GABAA receptors on vagal afferents to induce chloride influx and decrease firing rate 41 (FIG. 5), providing a connection between hepatic lipid accumulation and decreased HVAN activity.

It is counterintuitive that a decrease in HVAN activity causes hyperinsulinemia (FIGS. 2F, 2G, and 2J), yet surgical elimination of the HVAN limits obesity-induced hyperinsulinemia (FIG. 10). However, surgical vagotomy preserves activity of vagal afferent axons that terminate in the hindbrain. In fact, after vagotomy NTS terminating axons show normal spontaneous activity, normal signaling from the nodose ganglion to the NTS, and evoke normal postsynaptic excitatory currents in the NTS when electrically stimulated. Re-innervation of target tissues caudal to the severed vagus is minimal out to 45 weeks post-vagotomy.

Therefore, hepatic vagotomy eliminates the dysfunctional hepatocyte-vagal signaling in obesity, while preserving signaling from the HVAN above the surgical resection. Although hepatic vagotomy has been used extensively to investigate liver vagal denervation, several limitations must be acknowledged when interpreting this model. Alternatively, the improvements in glucose homeostasis in response to hepatic vagotomy (FIG. 1) are a result of interrupting non-vagal afferent signaling or partial loss of vagal efferent pancreatic innervation.

Studies manipulating the hepatic vagal nerve propose that afferent parasympathetic signals from the liver may affect pancreatic insulin release. Activity of the HVAN is inversely related to parasympathetic efferent nerve activity at the pancreas, which stimulates insulin release 4,5. Thus, portal glucose inhibits HVAN activity and increases pancreatic parasympathetic outflow to stimulate β-cell muscarinic 3 receptor (M3R) signaling and insulin release. Accordingly, vagotomy reduces glucose stimulated insulin secretion and basal hyperinsulinemia in obese rats by reducing cholinergic action on β-cells 4,46. Furthermore, cholinergic blockade decreases basal serum insulin concentrations in obese but not lean mice, suggesting that elevated basal pancreatic parasympathetic efferent tone underlies obesity-induced hyperinsulinemia.

The HVAN also regulates insulin sensitivity. Hepatic vagotomy acutely reduces insulin sensitivity in lean rats, decreasing skeletal muscle glucose clearance by 45%. In contrast, chronic hepatic vagotomy improves insulin sensitivity and glucose clearance in insulin resistant mice 6 (FIG. 1L). Portal glucose delivery decreases HVAN firing activity and skeletal muscle glucose clearance. Without wishing to limit the present invention to any particular theory or mechanism, it is believed that hepatic GABA production in obesity decreases HVAN activity to limit muscle glucose clearance and drive peripheral insulin resistance. Herein, the present invention identified enzymes involved in GABA production and transporters involved in hepatic GABA re-uptake and release as novel therapeutic targets for correcting the inherent metabolic disturbances in T2D.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-93. (canceled)

94. A method of reducing food intake in a monogastric animal or a human, said method comprising: administering to the monogastric animal or the human an effective amount of a composition that effectively depresses hepatic gamma-aminobutyric acid (GABA) production or release, wherein depressing hepatic GABA production or release causes the monogastric animal or human to reduce its food intake as compared to its food intake prior to being administered the composition.

95. (canceled)

96. The method of claim 94, wherein the method is for weight loss.

97. The method of claim 94, wherein the composition is a drug, a compound, or a molecule.

98. The method of claim 97, wherein the molecule is an anti-sense oligonucleotide.

99. The method of claim 94, wherein the composition inhibits GABA signaling on the hepatic vagal afferent nerve.

100. The method of claim 94, wherein the composition inhibits expression of or activity of GABA transaminase (GABA T) or inhibits GABA production.

101. (canceled)

102. The method of claim 94, wherein the composition inhibits GABA release.

103. The method of claim 94, wherein the composition inhibits expression or activity of GABA transporters that export hepatic GABA.

104. The method of claim 94, wherein the composition that inhibits GABA release inhibits expression of the Solute Carrier Family 6 Member 6 (SLC6A6) mRNA or the Solute Carrier Family 6 Member 8 (SLC6A8) mRNA.

105.-108. (canceled)

109. The method of claim 94, wherein the composition improves GABA re-uptake.

110. The method of claim 94, wherein the composition increases mRNA or protein expression of the Solute Carrier Family 6 Member 12 (SLC6A12) gene or the Solute Carrier Family 6 Member 13 (SLC6A13) gene.

111.-138. (canceled)
